(19) United States
(12) Patent Application Publication
(10) Pub. No.: US 2025/0277006 A1
(43) Pub. Date: Sep. 4, 2025

(54) FC BINDING POLYPEPTIDES

(71) Applicant: Cytiva Bioprocess R&D AB, Uppsala (SE)

(72) Inventors: Mats Arvid Ander, Uppsala (SE); Daniel Ivansson, Uppsala (SE); Andreas Lars, Magnus Jonsson, Uppsala (SE)

(21) Appl. No.: 18/689,737

(22) PCT Filed: Sep. 23, 2022
(86) PCT No.: PCT/EP2022/076493

§ 371 Date: Mar. 6, 2024

(30) Foreign Application Priority Data
Sep. 24, 2021 (GB) 2113626.2

(51) Int. Cl. C07K 14/31; C07K 1/22

(52) U.S. Cl. CPC C07K 14/31; C07K 1/22; C07K 2319/30

(57) ABSTRACT

The present disclosure relates to a class of engineered polypeptides having a binding affinity for the Fc region of immunoglobulins while exhibiting a significantly reduced binding affinity to the VH3 region of immunoglobulins. The present disclosure also relates to methods for isolating an immunoglobulin using said polypeptides as well as to related products, such as separation matrices.

### CROSS REFERENCE TO RELATED APPLICATIONS

This application claims the priority benefit of PCT/EP2022/076493, filed on Sep. 23, 2022, which claims the priority benefit of GB Application No. 2113626.2 filed on Sep. 24, 2021, the entire contents of which are incorporated herein by reference.

### SEQUENCE LISTING

The instant application contains a Sequence Listing which has been submitted electronically in ST.26 XML format and is hereby incorporated by reference in its entirety. Said ST.26 XML copy, created on Sep. 15, 2022, is named Sequence_Listing.xml and is 580,000 bytes in size.

### FIELD OF THE INVENTION

The present disclosure relates to a class of engineered polypeptides having a binding affinity for the Fc region of immunoglobulins while exhibiting a significantly reduced binding affinity to the VH3 region of immunoglobulins. The present disclosure also relates to methods for isolating an immunoglobulin using said polypeptides as well as to related products.

### BACKGROUND

Immunoglobulins represent the most prevalent biopharmaceutical products in either manufacture or development worldwide. The high commercial demand for and hence value of this particular therapeutic market has led to the emphasis being placed on pharmaceutical companies to maximize the productivity of their respective mAb manufacturing processes whilst controlling the associated costs.

Affinity chromatography is used in most cases, as one of the key steps in the purification of these immunoglobulin molecules, such as monoclonal or polyclonal antibodies. A particularly interesting class of affinity reagents is proteins capable of specific binding to invariable parts of an immunoglobulin molecule, such interaction being independent of the antigen-binding specificity of the antibody. Such reagents can be widely used for affinity chromatography recovery of immunoglobulins from different samples, such as but not limited to serum or plasma preparations or cell culture derived feed stocks. An example of such a protein is staphylococcal protein A (SpA), containing domains capable of binding to the Fc and Fab portions of IgG immunoglobulins from different species. These domains are commonly denoted as the E-, D-, A-, B- and C-domains.

SpA-based proteins have due to their high affinity and selectivity found a widespread use in the field of biotechnology, e.g. as ligands in affinity chromatography for capture and purification of antibodies as well as for detection or quantification. At present, SpA-based affinity medium is probably the most widely used affinity medium for isolation of monoclonal antibodies and their fragments from different samples including industrial cell culture supernatants. Accordingly, various matrices comprising protein A or protein A-derived ligands are commercially available, for example, in the form of MabSelect™ SuRe, MabSelect™ SuRe LX, MabSelect™ PrismA protein A and HiScreen Fibro™ PrismA from Cytiva, Uppsala, Sweden.

Certain Protein A and Protein A-derived ligands have binding affinity for both the Fc part of an antibody and for some VH domains of antibodies, in particular VH3. As a result, co-purification of product-related impurities such as half-antibodies and truncated variants may occur and require elution schemes which are complex and/or not sufficiently mild. When using these ligands in library applications aiming at finding novel binders against antibody domains, having inherent VH domain interactions can be problematic.

### SUMMARY OF THE INVENTION

It is an object of the present disclosure to provide Fc binding SpA-derived ligands which exhibit no, or significantly reduced affinity for the VH3 region of immunoglobulins. Such ligands could for example be used in methods for isolating an immunoglobulin and/or fragments thereof.

It is an object of the present disclosure to provide Fc binding SpA-derived ligands allowing for efficient isolation of immunoglobulins and/or fragments thereof while alleviating the abovementioned and other drawbacks of the prior art.

It is one object to provide Fc binding SpA-derived ligands enabling mild elution of purified antibodies.

It is a further object to provide Fc binding SpA-derived ligands which decreases or prevents co-purification of product-related impurities such as half-antibodies and truncated variants.

It is also a further object to provide Fc binding SpA-derived ligands which avoid competing interactions, e.g. to simplify identification of altered immunoglobulin variants showing changed sensitivity to pH-elution.

It is one object to provide Fc binding SpA-derived ligands which avoid competing interactions and therefore are useful in platform solutions for purification of all Fc containing products.

It is one object to provide Fc binding SpA-derived ligands which avoid competing binding interactions from VH3 and thus exhibit increased binding capacity for Fc containing products, in other words Fc binding SpA-derived ligands which are capable of binding more Fc containing products.

These and other objects which are evident to the skilled person from the present disclosure are met by different aspects of the invention as claimed in the appended claims and as generally disclosed herein.

Thus, in the first aspect of the disclosure, there is provided an Fc binding polypeptide derived from a Staphylococcus Protein A (SpA) or any domain thereof, wherein said polypeptide has binding affinity for an Fc region of an immunoglobulin, and has lower binding affinity for a VH3 region of trastuzumab compared to the binding affinity of SEQ ID NO:59 for the same VH3 region.

As an alternative, the Fc binding polypeptides as disclosed herein may be evaluated for binding affinity for a VH3 region of trastuzumab compared to the affinity of SEQ ID NO:58 for the same VH3 region. Thus, as an alternative, there is provided an Fc binding polypeptide derived from a Staphylococcus Protein A (SpA) or any domain thereof, wherein said polypeptide has binding affinity for an Fc region of an immunoglobulin, and has lower binding affinity for a VH3 region of trastuzumab compared to the binding affinity of SEQ ID NO:58 for the same VH3 region. Thus, it will be understood that in some embodiments, there is provided an Fc binding polypeptide derived from a Staphylococcus Protein A (SpA) or any domain thereof as disclosed herein, which has lower binding affinity for a VH3 region of trastuzumab compared to the binding affinity of SEQ ID NO:58 and of SEQ ID NO:59 for the same VH3 region.

The skilled person will appreciate that the Fc binding polypeptide derived from a Staphylococcus Protein A (SpA) or any domain thereof as disclosed herein may for example, but not necessarily, be derived from any one of domains A (SEQ ID NO:62), B (SEQ ID NO:61), C (SEQ ID NO:60), D (SEQ ID NO:63) and E (SEQ ID NO:64) of SpA or derivatives thereof, such as domain Z (SEQ ID NO:59) or variants thereof, such as SEQ ID NO:58. The Fc binding polypeptide derived from a SpA domain may thus be a derivative, a mutant, a variant or a fragment of an SpA domain as defined above.

The Fc binding polypeptides of the present disclosure thus are characterized by their retained inherent ability to bind to the Fc (fragment crystallizable region) of antibodies/immunoglobulins, in particular to the Fc region of the antibody subclasses IgG1, IgG2 and IgG4, and their reduced or abolished binding affinity for the VH3 region of the antibody trastuzumab. The skilled person appreciates that similar effect will be observed for antibodies or fragments thereof containing the same or similar VH3 region as trastuzumab.

As used herein the term VH3 in the context of binding affinity for VH3 refers to the VH3 region of the antibody trastuzumab (trade names Herceptin™ (Roche), Trazimera™ (Pfizer)) which region comprises the amino acid residues in the following positions according to the Kabat numbering system: H15:G; H17:S; H19:R; H57:T; H59:Y; H64:K; H65:G; H66:R; H68:T; H70:S; H81:Q; H82a:N and H82b:S. For avoidance of any doubt, the term “VH3 region of trastuzumab” and the term “VH3” (without any additional specification) are used to refer to the VH3 region of the antibody trastuzumab.

The present inventors have surprisingly found that by introducing mutations in positions X₃₃, X₄₀ and X₅₁ of Fc binding polypeptides derived from SpA or domains thereof, the binding affinity for VH3 of trastuzumab is significantly reduced or abolished. X₃₃, X₄₀ and X₅₁ refer to positions 33, 40 and 51 in a SpA domain, such as SEQ ID NO:58 and 59. Thus, the presently identified mutations in positions X₃₃, X₄₀ and X₅₁ of the Fc binding polypeptides as defined herein significantly reduce or abolish binding affinity for said VH3. The Fc binding polypeptides comprising amino acids as defined herein in positions X₃₃, X₄₀ and X₅₁ loose at least a significant part of their affinity for VH3 while retaining the affinity for the Fc region.

According to a second aspect, there is provided an Fc binding polypeptide comprising a sequence A, which Sequence A consists of an amino acid sequence selected from i), ii) and iii),
wherein i), ii) and iii) are defined as follows:
v) EX₂₅QX₂₇X₂₈X₂₉X₃₀₁X₃₂X₃₃ LX₃₅X₃₆X₃₇PSX₄₀SX₄₂X₄₃X₄₄LX₄₆EAX₄₉X₅₀X₅₁N₂X₅₃ X₅₄
   wherein, independently from each other,
X₂₅ is selected from E and D;
X₂₇ is selected from R and H;
X₂₈ is selected from N, A, S, H and W;
X₂₉ is selected from A, G and K;
X₃₀ is selected from F and A;
X₃₂ is selected from Q and H;
X₃₅ is selected from K, R and H;
X₃₆ is selected from D and H;
X₃₇ is selected from D and E;
X₄₂ is selected from A, K, L, R and T;
X₄₃ is selected from N, E, A, K and S;
X₄₄ is selected from L, I and V;
X₄₆ is selected from A, G and K;
X₄₉ is selected from K, Q and R;
X₅₀ is selected from K and R;
X₅₃ is selected from D, E and K; and
X₅₄ is selected from A and S;
ii) an amino acid sequence which has at least 83% identity to a sequence defined by i)
iii) an amino acid sequence which has at least 70% identity to any sequence selected from the group consisting of: residues 24-54 in SEQ ID NO:58, residues 24-54 in SEQ ID NO:59, residues 24-54 in SEQ ID NO:60, residues 24-54 in SEQ ID NO:61, residues 24-54 in SEQ ID NO:62, residues 27-57 in SEQ ID NO:63 and residues 22-52 in SEQ ID NO:64,
wherein additionally, in each of i), ii) and iii) independently from each other,
X₃₃ is selected from T, S, G, Q, A, E, H, R, P, D, K and N;
X₄₀ is selected from E, G, R, D, K, Q, N, H and S, and
X₅₁ is selected from L, V, S, I, R and G;
   with the proviso that when X₅₁ is L then X₃₃X₄₀ is selected from AD, HK, EG, ER, GR, AK, AR, PK, RR and KK and when X₅₁ is G then X₃₃X₄₀ is TK.

In one embodiment of the second aspect, said Fc binding polypeptide comprises a sequence A, which Sequence A consists of an amino acid sequence selected from i), ii) and iii) as defined above, and wherein additionally, in each of i), ii) and iii) independently from each other,
X₃₃ is selected from T, S, G, Q, A, E, H, R, P, D, K and N;
X₄₀ is selected from E, G, R, D, K, Q, N, H and S, and
X₅₁ is selected from L, V, S, I, R and G;
with the proviso that when X₅₁ is L then X₃₃X₄₀ is selected from AD, HK, EG, ER, GR, AK, AR, PK, RR and KK and when X₅₁ is G then X₃₃X₄₀ is TK;
   and
wherein said Fc binding polypeptide has a lower affinity for VH3 region of trastuzumab than SEQ ID NO:59.

As explained above, the Fc binding polypeptides as disclosed herein may be evaluated for binding affinity for a VH3 region of trastuzumab compared to the affinity of SEQ ID NO:58 for the same VH3 region. Thus, as an alternative, there is provided an Fc binding polypeptide comprising a sequence A, which Sequence A consists of an amino acid sequence selected from i), ii) and iii) as defined above, and wherein additionally, in each of i), ii) and iii) independently from each other,
X₃₃ is selected from T, S, G, Q, A, E, H, R, P, D, K and N;
X₄₀ is selected from E, G, R, D, K, Q, N, H and S, and
X₅₁ is selected from L, V, S, I, R and G;
with the proviso that when X₅₁ is L then X₃₃X₄₀ is selected from AD, HK, EG, ER, GR, AK, AR, PK, RR and KK and when X₅₁ is G then X₃₃X₄₀ is TK.

As an additional alternative, there is provided an Fc binding polypeptide comprising a sequence A, which Sequence A consists of an amino acid sequence selected from i), ii) and iii) as defined above, and wherein additionally, in each of i), ii) and iii) independently from each other,
X₃₃ is selected from T, S, G, Q, A, E, H, R, P, D, K and N;
X₄₀ is selected from E, G, R, D, K, Q, N, H and S, and
X₅₁ is selected from L, V, S, I, R and G;
   with the proviso that when X₅₁ is L then X₃₃X₄₀ is selected from AD, HK, EG, ER, GR, AK, AR, PK, RR and KK and when X₅₁ is G then X₃₃X₄₀ is TK;
   and
wherein said Fc binding polypeptide has lower binding affinity for a VH3 region of trastuzumab compared to the binding affinity of SEQ ID NO:58 for the same VH3 region.

Again, it will be understood that in some embodiments, there is provided an Fc binding polypeptide as disclosed herein, which has lower binding affinity for a VH3 region of trastuzumab compared to the binding affinity of SEQ ID NO:58 and of SEQ ID NO:59 for the same VH3 region. It will be understood by the skilled person that the comparisons of binding affinity for the VH3 region of trastuzumab exhibited by the Fc binding polypeptides of the present disclosure may be made using SEQ ID NO:59 and/or SEQ ID NO:58 as a reference.

To clarify, it will be appreciated that the % identity in ii) and iii) does not relate to X₃₃, X₄₀ and X₅₁. Thus, the amino residues in positions X₃₃, X₄₀ and X₅₁ are as defined above in Fc binding polypeptides encompassed by the definition according to ii) and iii).

The above definition of a class of sequence related, Fc binding polypeptides is based on a statistical analysis of a number of Fc binding polypeptide variants of a parent scaffold, that were selected for the properties of at least maintained affinity for Fc and significantly reduced affinity for VH3 in selection experiments.

It is also envisioned that said Fc binding polypeptide will exhibit a lower affinity for VH3 region of trastuzumab than otherwise identical Fc binding polypeptide wherein X₃₃X₄₀X₅₁ is SQL. Thus a comparison may be made between any otherwise identical Fc binding polypeptide wherein X₃₃X₄₀X₅₁ is SQL and the Fc binding polypeptide of the present disclosure. In one embodiment, said Fc binding polypeptide has a lower affinity for the VH3 region of trastuzumab than an otherwise identical Fc binding polypeptide wherein X₃₃X₄₀X₅₁ is SQL. In yet another embodiment, said Fc binding polypeptide has a lower affinity for the VH3 region of trastuzumab than an otherwise identical Fc binding polypeptide wherein X₃₃X₄₀X₅₁ is SQL, and than SEQ ID NO:59.

It is also envisioned that said Fc binding polypeptide will exhibit a lower affinity for VH3 region of trastuzumab than otherwise identical Fc binding polypeptide wherein X₃₃X₄₀X₅₁ is SVL. Thus, in an alternative embodiment, a comparison may be made between any otherwise identical Fc binding polypeptide wherein X₃₃X₄₀X₅₁ is SVL and the Fc binding polypeptide of the present disclosure. In an alternative embodiment, said Fc binding polypeptide has a lower affinity for the VH3 region of trastuzumab than an otherwise identical Fc binding polypeptide wherein X₃₃X₄₀X₅₁ is SVL. In yet another embodiment, said Fc binding polypeptide has a lower affinity for the VH3 region of trastuzumab than an otherwise identical Fc binding polypeptide wherein X₃₃X₄₀X₅₁ is SVL, and than SEQ ID NO:58.

The Fc binding polypeptides may comprise the identified sequence A, which corresponds to the region which interacts and binds to VH3 of the parent scaffold. This region constitutes two alpha helices, namely helix 2 and helix 3, within a three-helical bundle protein domain. Helix 1 and helix 2 of this three-helical bundle protein constitute a binding surface for interaction with the Fc region of antibodies.

As the skilled person will realize, the function of any polypeptide is dependent on the tertiary structure of the polypeptide. It is therefore possible to make minor changes to the sequence of amino acids in a polypeptide without affecting the function thereof. Thus, the disclosure encompasses modified variants of the Fc binding polypeptide, which are such that the Fc binding characteristics are retained, while the binding characteristics for VH3 are significantly reduced or abolished.

The modification of VH3 binding affinity identified by the present inventors, is applicable to Fc binding polypeptides based on the different three-helical domains of Protein A from Staphylococcus aureus, such as any one of the domains A (SEQ ID NO:62), B (SEQ ID NO:61), C (SEQ ID NO:60), D (SEQ ID NO:63) and E (SEQ ID NO:64), in particular domain B, and derivatives thereof. In particular, said modification may be applicable to Fc binding polypeptides based on the three-helical bundle protein domain Z (SEQ ID NO:59), which domain Z is derived from domain B of staphylococcal Protein A or to the variant SEQ ID NO:58. Such Fc binding polypeptides have been described in: WO2003080655, EP230869A2, Sjödahl, Eur J Biochem 1977 September; 78(2):471-90, WO2008039141, WO2015005859, U.S. Ser. No. 10/308,690, WO2016079033, WO2017194596, U.S. Pat. Nos. 8,859,726, 9,187,555, 9,683,013, U.S. Ser. No. 10/189,891, U.S. Pat. No. 9,663,558, WO2016079034, JP2006304633A, WO2007097361, EP1992692A1, EP2202310A2, WO2012083425, WO2012086660, US20120208234, EP2495254A1, WO2012133342, WO2013109302A2, WO2015034000, WO2015034056, CN105481954A, WO2016152946A1, WO2017009421, WO2017014261, WO2017014260, WO2018009006, WO2018029158, WO2018029157, WO2019030156, CN109721645A, WO2019093439, WO2020040307 and WO2020157281, which are hereby incorporated by reference.

Thus, the present inventors envision that the mutations in positions X₃₃, X₄₀ and X₅₁ as disclosed herein may be introduced into any one of the previously known Fc binding polypeptides to achieve the effect of significantly reduced or abolished binding affinity for VH3, while retaining the Fc binding characteristics.

Also encompassed by the present disclosure is an Fc binding polypeptide comprising a Sequence A with at least 83% identity to a polypeptide as defined in i). In some embodiments, the polypeptide may comprise a sequence which is at least 87%, such as at least 90%, such as at least 93%, such as at least 96% identical to a polypeptide as defined in i). For example, it is possible that an amino acid residue belonging to a certain functional grouping of amino acid residues (e.g. hydrophobic, hydrophilic, polar etc.) could be exchanged for another amino acid residue from the same functional group. In some embodiments, such changes may be made in any position except for X₃₃, X₄₀ and X₅₁ of the sequence of the Fc binding polypeptide as disclosed herein. In other embodiments, such changes may be made only in the non-variable positions, also denoted scaffold amino acid residues. In such cases, changes are not allowed in the variable positions, i.e. positions denoted with an “X” in sequence i).

Also encompassed by the present disclosure is an Fc binding polypeptide comprising a Sequence A which Sequence A has at least 70% identity to any sequence selected from the group consisting of: residues 24-54 in SEQ ID NO:58, residues 24-54 in SEQ ID NO:59, residues 24-54 in SEQ ID NO:60, residues 24-54 in SEQ ID NO:61, residues 24-54 in SEQ ID NO:62, residues 27-57 in SEQ ID NO:63 and residues 22-52 in SEQ ID NO:64. In some embodiments, the polypeptide may comprise a sequence which is at least 74%, such as at least 77%, such as at least 80%, such as at least 83%, such as at least 87%, such as at least 90%, such as at least 93%, such as at least 96%, identical to any sequence selected from the group consisting of: residues 24-54 in SEQ ID NO:58, residues 24-54 in SEQ ID NO:59, residues 24-54 in SEQ ID NO:60, residues 24-54 in SEQ ID NO:61, residues 24-54 in SEQ ID NO:62, residues 27-57 in SEQ ID NO:63 and residues 22-52 in SEQ ID NO:64. In particular embodiments, said sequence is selected from the group consisting of: residues 24-54 in SEQ ID NO:58, residues 24-54 in SEQ ID NO:59, and residues 24-54 in SEQ ID NO:61, such as the group consisting of: residues 24-54 in SEQ ID NO:58 and residues 24-54 in SEQ ID NO:59. In one embodiment, said sequence is residues 24-54 in SEQ ID NO:58.

The term “% identity”, as used throughout the disclosure, may for example be calculated as follows. The query sequence is aligned to the target sequence using the CLUSTAL W algorithm (Thompson et al, Nucleic Acids Research, 22: 4673-4680 (1994)). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % identity.

In one embodiment, the Fc binding polypeptide comprising a Sequence A, which consists of an amino acid sequence selected from i), ii) and iii) comprises amino acid residues selected independently from T, S, G, Q, A, E, H, R, P, D, K and N for X₃₃; from E, G, R, D, K, Q, N, H and S for X₄₀; and L, V, S, I, R and G for X₅₁; and in the case wherein X₅₁ is L then X₃₃X₄₀ is selected from AD, HK, EG, ER, GR, AK, AR, PK, RR and KK and when X₅₁ is G then X₃₃X₄₀ is TK.

In one particular embodiment provided herein, in each of i), ii) and iii) independently from each other,
X₃₃ is selected from T, S, G, Q, A, E, H, R, P, D, K and N;
X₄₀ is selected from E, G, R, D, K, Q, N, H and S; and
X₅₁ is selected from L, V, S, I and R;
   with the proviso that when X₅₁ is L, X₃₃X₄₀ is selected from AD, HK, EG, ER, GR, AK, AR, PK, RR and KK and wherein said Fc binding polypeptide has a lower affinity for VH3 of trastuzumab than SEQ ID NO:58.

As explained above, the comparison may alternatively be made with an otherwise identical Fc binding polypeptide wherein X₃₃X₄₀X₅₁ is SVL; or with both an otherwise identical Fc binding polypeptide wherein X₃₃X₄₀X₅₁ is SVL and SEQ ID NO:58.

In one embodiment of the Fc binding polypeptides as disclosed herein, said Sequence A fulfills the criteria i) and ii), or the criteria i) and iii), or the criteria ii) and iii). In one embodiment, said Sequence A fulfills all criteria i), ii) and iii).

In one embodiment according to the second aspect as disclosed herein, X₃₃ is selected from T, S, G, Q, A, E, H, R, P, D and K, such as selected from T, S, G, Q, A, H, R, P, D and K, such as selected from T, S, G, Q, A, H, R, P and D or selected from T, S, G, Q, A, H, R, P and K; such as selected from T, S, G, Q, A, H, R and P; such as selected from T, S, G, Q, A, H and P or selected from T, G, Q, A, H, R and P; such as selected from T, G, Q, P, A and H; such as selected from T, G, Q, H and P; such as selected from T, G, Q and P or selected from G, Q, H and P or selected from T, G, H and P; such as the group selected from G and P; such is G. In one embodiment, X₃₃ is selected from T, S, G, Q, A, E and H, such as selected from T, S, G, Q, A and E, such as selected from S, G, Q and A. In one embodiment, X₃₃ is selected from S, G, A, E and H, such as selected from S, G, A and E. In one embodiment, X₃₃ is selected from A, S and E. In one embodiment, X₃₃ is selected from A and S or selected from A and E or selected from S and E. In one embodiment, X₃₃ is S.

In one embodiment, X₄₀ is selected from E, G, R, D, K, Q, N and S or selected from E, G, R, D, K, Q, H and S or selected from G, R, D, K, Q, N, H and S, such as selected from G, R, D, K, Q, and S, such as selected from G, R, D, K and Q; such as selected from R, D, K and Q; such as selected from R, K and Q; such as selected from R and K; such as is R or is K. In one embodiment, X₄₀ is selected from E, G, R, D, K and Q, such as selected from E, G, R, D, K and Q, such as selected from E, G, R, D and K or selected from E, G, R, D and Q. In one embodiment, X₄₀ is selected from G, R, E and D. In one embodiment, X₄₀ is selected from G, R and D. In one embodiment, X₄₀ is selected from G, R and K. In one embodiment, X₄₀ is selected from G and R. In one embodiment, X₄₀ is G. In one embodiment, X₄₀ is R.

In one embodiment, X₅₁ is selected from L, V, I, R and S. In one embodiment X₅₁ is selected from L, I, R and S. In one embodiment, X₅₁ is selected from L, V, I and R. In one embodiment, X₅₁ is selected from I, V and R; or is selected from L, I and R; or is selected from V, L and I; or is selected from R, I and L. In one embodiment, X₅₁ is selected from V and L; or is selected from V and I; or is selected from V and R; or is selected from L and I; or is selected from L and R; or is selected from I and R. In one embodiment, X₅₁ is V or L or I or R. In one embodiment, X₅₁ is 1. In one embodiment, X₅1 is V.

In one embodiment, X₅₁ is selected from L, V, S, I and R with the proviso that when X₅₁ is L then X₃₃X₄₀ is selected from AD, HK, EG, ER, GR, AK, AR, PK, RR and KK. In another embodiment, X₅₁ is selected from L, V, S, I and R with the proviso that when X₅₁ is L then X₃₃X₄₀ are AD or HK.

In one embodiment of the Fc binding polypeptide, at least one, such as two or all three, of amino acid residues X₃₃, X₄₀ and X₅₁ is/are (an) uncharged amino acid residue(s). In one embodiment, the amino acid residue in X₃₃ is an uncharged amino acid. In one embodiment, the amino acid residue in X₄₀ is an uncharged amino acid.

In one particular embodiment there is provided an Fc binding polypeptide, wherein at least two of the amino acid residues in positions X₃₃, X₄₀ and X₅₁ are mutated compared to SEQ ID NO:58 or 59. In particular, positions X₄₀ and X₅₁ may be mutated compared to SEQ ID NO:58 or 59.

In a more specific embodiment defining a sub-class of Fc binding polypeptides as disclosed herein, X₃₃X₄₀X₅₁ are selected from the group consisting of ADL, AGR, AKL, ARI, ARL, DKI, DNS, DRV, EGL, ERL, GDR, GDV, GER, GGI, GGS, GNI, GQI, GRI, GRL, GRR, HKI, HKL, HQS, HRI, HSR, HSV, KEI, KKL, KRI, NHS, PDR, PHR, PHS, PKL, PKS, PQR, PSI, QDI, QDV, QNS, QQI, QRI, QRV, RQR, ROS, RRI, RRL, SGV, SRR, SRS, SRV, TER, TKG, TKI, TQI, TRR and TSR, such as the group consisting of ADL, AGR, AKL, ARI, ARL, DKI, DNS, DRV, EGL, ERL, GDR, GDV, GER, GGI, GNI, GRI, GRL, GRR, HKI, HKL, HQS, HRI, HSR, HSV, KEI, KKL, KRI, NHS, PDR, PHR, PHS, PKL, PKS, PQR, PSI, QDI, QDV, QNS, QQI, QRI, QRV, RQR, ROS, RRI, RRL, SGV, SRR, SRS, SRV, TER, TKI, TQI, TRR and TSR. In one embodiment, X₃₃X₄₀X₅₁ are selected from the group consisting of GGS, GGI, SRV, GRL, QRI, ARI, SGV, TER, GER, ADL, AGR, EGL, HKL, GDV, GQI and QDI, or the group consisting of GGI, SRV, GRL, QRI, ARI, SGV, TER, GER, ADL, AGR, EGL, HKL, GDV and QDI. In another embodiment, X₃₃X₄₀X₅₁ are selected from the group consisting of GGI, SRV, GRL, QRI, ARI, SGV, TER, AGR, EGL, HKL and GDV. In one embodiment, X₃₃X₄₀X₅₁ are selected from the group consisting of GGI, ARI, SGV, AGR, EGL and HKL. In one embodiment, X₃₃X₄₀X₅₁ are selected from the group consisting of GGI, ARI, SGV, AGR and EGL. In one embodiment, X₃₃X₄₀X₅₁ are selected from the group consisting of GGI, SRV, GRL, QRI, ARI, SGV, TER, AGR, EGL and GDV. In one embodiment, X₃₃X₄₀X₅₁ are selected from the group consisting of GGI, SRV, QRI, ARI, SGV, AGR and GDV. In one embodiment, X₃₃X₄₀X₅₁ are selected from the group consisting of SRV, GRL, QRI, ARI, SGV, TER, GER, ADL, AGR, EGL, HKL, GDV, GQI and QDI. In one embodiment, X₃₃X₄₀X₅₁ are selected from the group consisting of GGI, GRL, ARI, SGV, AGR and EGL. In one embodiment, X₃₃X₄₀X₅₁ are selected from the group consisting of ARI, SGV, AGR and EGL. In another embodiment, X₃₃X₄₀X₅₁ are selected from the group consisting of SGV, AGR and EGL. In one embodiment, X₃₃X₄₀X₅₁ are selected from the group consisting of ARI, AGR and EGL. In one embodiment, X₃₃X₄₀X₅₁ are selected from the group consisting of ARI, SGV and EGL. In one embodiment, X₃₃X₄₀X₅₁ are selected from the group consisting of ARI, SGV and AGR.

In one embodiment, X₃₃X₄₀X₅₁ are selected from the group consisting of ARI and SVG. In one embodiment, X₃₃X₄₀X₅₁ are selected from the group consisting of ARI and AGR, or the group consisting of ARI and EGL. In one embodiment, X₃₃X₄₀X₅₁ are selected from the group consisting of SGV and AGR. In one embodiment, X₃₃X₄₀X₅₁ are selected from the group consisting of SGV and EGL. In one embodiment, X₃₃X₄₀X₅₁ are selected from the group consisting of AGR and EGL. In one embodiment, X₃₃X₄₀X₅₁ is ARI or SGV or AGR or EGL. In one particular embodiment, X₃₃X₄₀X₅₁ is SGV.

Herein, “Xₙ” and “Xₘ” are used to indicate amino acid residues in positions n and m corresponding to positions n and m in e.g. SEQ ID NO 59 as defined herein, wherein n and m are integers which indicate the position of an amino acid residue within said sequence as counted from the N-terminal end of said sequence. For example, X₂₄ and X₁₃ indicate the amino acid residue in positions that corresponds to positions twenty-four and thirteen, respectively, from the N-terminal end of e.g. SEQ ID NO 59 as defined herein, in an alignment. To clarify the X numbering as used herein is based on the full-length scaffold comprising 58 amino acid residues. Hence X₂₄ is to be understood as being the 24^{th} amino acid residue in the 58-mer. The skilled person is able to make alignments of sequences to determine the position of an amino acid in accordance with the above.

As described in detail in the experimental section to follow, the selection of Fc binding polypeptide variants has led to the identification of a number of individual amino acid sequences according to Sequence A as defined herein. These sequences constitute individual embodiments of sequence i) according to this aspect. The sequences of individual amino acid sequences according to Sequence A correspond to amino acid positions 24-54 in SEQ ID NO:1-57 and SEQ ID NO:208-264 presented in FIG. 10, as well as SEQ ID NO:269-305. Hence, in one embodiment according to the second aspect as disclosed herein, there is provided an Fc binding polypeptide wherein sequence i) corresponds to the sequence from position 24 to position 54 in a sequence selected from the group consisting of SEQ ID NO:1-57, 208-264 and 269-305, such as SEQ ID NO:1-56 and 208-263. In one embodiment, sequence i) corresponds to the sequence from position 24 to position 54 in a sequence selected from the group consisting of SEQ ID NO: SEQ ID NO:2-57, 209-264 and 269-305. In one embodiment, sequence i) corresponds to the sequence from position 24 to position 54 in a sequence selected from the group consisting of SEQ ID NO:1-16 and 208-223 or the group consisting of SEQ ID NO:2-14, 16, 209-221 and 223. In one embodiment, sequence i) corresponds to the sequence from position 24 to position 54 in a sequence selected from the group consisting of SEQ ID NO: SEQ ID NO:2-16 and 209-223. In one embodiment, sequence i) corresponds to the sequence from position 24 to position 54 in a sequence selected from the group consisting of SEQ ID NO:2, 3, 4, 5, 6, 7, 8, 11, 12, 14, 209, 210, 211, 212, 213, 214, 215, 218, 219 and 221. In one embodiment, sequence i) corresponds to the sequence from position 24 to position 54 in a sequence selected from the group consisting of SEQ ID NO:2, 4, 6, 7, 11, 12, 209, 211, 213, 214, 218 and 219. In one embodiment, sequence i) corresponds to the sequence from position 24 to position 54 in a sequence selected from the group consisting of SEQ ID NO:6, 7, 11, 12, 213, 214, 218 and 219. In one embodiment, sequence i) corresponds to the sequence from position 24 to position 54 in a sequence selected from the group consisting of SEQ ID NO:6, 7, 11, 213, 214 and 218. In one embodiment, sequence i) corresponds to the sequence from position 24 to position 54 in a sequence selected from the group consisting of SEQ ID NO:7 and 214.

In one embodiment according to the second aspect as disclosed herein, there is provided an Fc binding polypeptide wherein sequence i) corresponds to the sequence from position 24 to position 54 in a sequence selected from the group consisting of SEQ ID NO:208-264 and 269-305, such as SEQ ID NO:208-263. In one embodiment, sequence i) corresponds to the sequence from position 24 to position 54 in a sequence selected from the group consisting of SEQ ID NO:209-264 and 269-305. In one embodiment, sequence i) corresponds to the sequence from position 24 to position 54 in a sequence selected from the group consisting of SEQ ID NO:208-223 or the group consisting of SEQ ID NO:209-221 and 223. In one embodiment, sequence i) corresponds to the sequence from position 24 to position 54 in a sequence selected from the group consisting of SEQ ID NO:209-223. In one embodiment, sequence i) corresponds to the sequence from position 24 to position 54 in a sequence selected from the group consisting of SEQ ID NO:209, 210, 211, 212, 213, 214, 215, 218, 219 and 221. In one embodiment, sequence i) corresponds to the sequence from position 24 to position 54 in a sequence selected from the group consisting of SEQ ID NO:209, 211, 213, 214, 218 and 219. In one embodiment, sequence i) corresponds to the sequence from position 24 to position 54 in a sequence selected from the group consisting of SEQ ID NO:213, 214, 218 and 219. In one embodiment, sequence i) corresponds to the sequence from position 24 to position 54 in a sequence selected from the group consisting of SEQ ID NO:213, 214 and 218. In one embodiment, sequence i) corresponds to the sequence from position 24 to position 54 in SEQ ID NO:214.

In one embodiment according to the second aspect as disclosed herein, there is provided an Fc binding polypeptide wherein sequence i) corresponds to the sequence from position 24 to position 54 in a sequence selected from the group consisting of SEQ ID NO:1-57, such as SEQ ID NO:1-56. In one embodiment, sequence i) corresponds to the sequence from position 24 to position 54 in a sequence selected from the group consisting of SEQ ID NO:2-57. In one embodiment, sequence i) corresponds to the sequence from position 24 to position 54 in a sequence selected from the group consisting of SEQ ID NO:1-16 or the group consisting of SEQ ID NO:2-14 and 16. In one embodiment, sequence i) corresponds to the sequence from position 24 to position 54 in a sequence selected from the group consisting of SEQ ID NO:2-16. In one embodiment, sequence i) corresponds to the sequence from position 24 to position 54 in a sequence selected from the group consisting of SEQ ID NO:2, 3, 4, 5, 6, 7, 8, 11, 12 and 14. In one embodiment, sequence i) corresponds to the sequence from position 24 to position 54 in a sequence selected from the group consisting of SEQ ID NO:2, 4, 6, 7, 11 and 12. In one embodiment, sequence i) corresponds to the sequence from position 24 to position 54 in a sequence selected from the group consisting of SEQ ID NO:6, 7, 11 and 12. In one embodiment, sequence i) corresponds to the sequence from position 24 to position 54 in a sequence selected from the group consisting of SEQ ID NO:6, 7 and 11. In one embodiment, sequence i) corresponds to the sequence from position 24 to position 54 in SEQ ID NO:7.

In some embodiments of the present disclosure, the polypeptide derived from SpA or Sequence A as defined above “forms part of” a three-helix bundle protein domain. This is understood to mean that the sequence of the SpA derived polypeptide or Sequence A is “inserted” into or “grafted” onto the sequence of the original three-helix bundle domain, such that the grafted sequence replaces a similar structural motif in the original domain. For example, without wishing to be bound by theory, the Sequence A is thought to constitute two of the three helices of a three-helix bundle and can therefore replace such a two-helix motif within any three-helix bundle. As the skilled person will realize, the replacement of two helices of the three-helix bundle domain by the two Sequence A helices has to be performed so as not to affect the basic structure of the polypeptide. That is, the overall folding of the Ca backbone of the polypeptide according to this embodiment of the invention is substantially the same as that of the three-helix bundle protein domain of which it forms a part, e.g. having the same elements of secondary structure in the same order etc. Thus, a Sequence A according to the disclosure “forms part” of a three-helix bundle domain if the polypeptide according to this embodiment has the same fold as the original domain, implying that the basic structural properties are shared, those properties e.g. resulting in similar CD spectra. The skilled person is aware of other parameters that are relevant.

Thus, in one embodiment the SpA derived polypeptide or Sequence A forms part of a three-helix bundle protein domain. In some embodiments, said Sequence A forms, or essentially forms, part of two helices with an interconnecting loop, within said three-helix bundle protein domain. In particular, said three-helix bundle protein domain may be selected from domains from bacterial receptor proteins, in other words from bacterial receptor domains. Non-limiting examples of such domains are the five different three-helical domains of Protein A from Staphylococcus aureus, such as domain A, B, C, D and E, in particular domain B and derivatives thereof and domain C and derivatives thereof. An example of a derivative of said domain B is domain Z. Another example of a derivative of domain C is domain C with the mutation G29A. In one embodiment, said three-helix bundle protein domain is selected from domains of protein A from Staphylococcus aureus or derivatives thereof. In some embodiments, the three-helical bundle protein domain is a variant of domain Z, which is derived from domain B of staphylococcal Protein A. In some embodiments, the three-helical bundle protein domain is a domain C comprising the mutation G29A.

Thus, in one embodiment, there is provided an Fc binding polypeptide as defined herein, further comprising a Sequence B arranged N-terminally of said Sequence A, which Sequence B consists of an amino acid sequence selected from iv) and v), and wherein iv) and v) are defined as follows:
iv) X₈X₉X₁₀X₁₁AFYX₁₅IX₁₇X₁₈X₁₉PX₂₁LX₂₃,
   wherein, independently from each other,
X₈ is selected from E, D and A;
X₉ is selected from Q, A, L, W, E, V, K, T and H;
X₁₀ is selected from Q and H;
X₁₁ is selected from N, A, S, E, K, H, Q, Y, T, F, L, W, I, M, V and R;
X₁₅ is selected from E, H and Q;
X₁₇ is selected from L and H;
X₁₈ is selected from H, N and K;
X₁₉ is selected from L and M;
X₂₁ is selected from N, Y and S;
X₂₃ is selected from T and N;
v) an amino acid sequence which has at least 75% identity to a sequence defined by iv).

In some embodiments where the sequence of Fc binding polypeptide as disclosed herein forms part of a three-helix bundle protein domain, it essentially forms part of at least three helices and the interconnecting loop between helix 1 and helix 2 (referred to as loop 1 (L1)) and the interconnecting loop between helix 2 and helix 3 (referred to as loop 1 (L2)). In this case, Sequence B essentially corresponds to helix 1 and the and L1. As used herein, the term “essentially corresponds to” is to be interpreted as making up the main part of the structure, in other words a few amino acid residues (for example, but not limited to, 1, 2, 3, 4 or 5 residues) may be missing compared to the original three-helix bundle protein domain.

As discussed above, polypeptides comprising minor changes as compared to the above amino acid sequences without largely affecting the tertiary structure and the function thereof are also within the scope of the present disclosure. Thus, in some embodiments, sequence v) has at least 87% identity, such as at least 90% identity, such as at least 93% identity, such as at least 96% identity with the sequence iv).

As explained above, “Xₙ” and “Xₘ” are used to indicate amino acid residues in positions n and m corresponding to positions n and m in SEQ ID NO 59 as defined above, wherein n and m are integers which indicate the position of an amino acid residue within said sequence as counted from the N-terminal end of said sequence. Again, the X numbering as used herein is based on the full-length scaffold comprising 58 amino acid residues. Hence X₈ is to be understood as being the 8th amino acid residue in the 58-mer. The skilled person is able to make alignments of sequences to determine the position of an amino acid in accordance with the above.

Thus in yet another embodiment, there is provided an Fc binding polypeptide as defined herein, which comprises a binding module sequence C, which Sequence C comprises or consists of Sequence A as defined herein and Sequence B as defined herein, in the following order from the N-terminus to the C-terminus
[Sequence B]-[Sequence A]
   or comprises or consists of any amino acid sequence which has at least 70% identity to any sequence selected from the group consisting of: residues 8-54 in SEQ ID NO:58, residues 8-54 in SEQ ID NO:59, residues 8-54 in SEQ ID NO:60, residues 8-54 in SEQ ID NO:61, residues 8-54 in SEQ ID NO:62, residues 11-57 in SEQ ID NO:63 and residues 6-52 in SEQ ID NO:64 wherein, independently from each other,
X₃₃ is selected from T, S, G, Q, A, E, H, R, P, D, K and N;
X₄₀ is selected from E, G, R, D, K, Q, N, H and S, and
X₅₁ is selected from L, V, S, I, R and G;
   with the proviso that when X₅₁ is L then X₃₃X₄₀ is selected from AD, HK, EG, ER, GR, AK, AR, PK, RR and KK and when X₅₁ is G then X₃₃X₄₀ is TK.

In one embodiment, said sequence C is selected from the group consisting of: residues 8-54 in SEQ ID NO:58, residues 8-54 in SEQ ID NO:59, and residues 8-54 in SEQ ID NO:61, such as the group consisting of: residues 8-54 in SEQ ID NO:58 and residues 8-54 in SEQ ID NO:59. In one embodiment, said sequence is residues 8-54 in SEQ ID NO:58.

In one embodiment, said sequence C has at least 70% identity to any sequence selected from the group consisting of: residues 8-54 in SEQ ID NO:58, residues 8-54 in SEQ ID NO:59, residues 8-54 in SEQ ID NO:60, residues 8-54 in SEQ ID NO:61, residues 8-54 in SEQ ID NO:62, residues 11-57 in SEQ ID NO:63 and residues 6-52 in SEQ ID NO:64, has at least 72%, such as at least 74%, such as at least 76%, such as at least 78%, such as at least 80%, such as at least 82%, such as at least 85%, such as at least 87%, such as at least 89%, such as at least 91%, such as at least 93%, such as at least 85%, such as at least 97%, identity to any sequence selected from the group consisting of: residues 8-54 in SEQ ID NO:58, residues 8-54 in SEQ ID NO:59, residues 8-54 in SEQ ID NO:60, residues 8-54 in SEQ ID NO:61, residues 8-54 in SEQ ID NO:62, residues 11-57 in SEQ ID NO:63 and residues 6-52 in SEQ ID NO:64. As previously explained, the % identity does not apply to the amino acid residues in positions X₃₃, X₄₀ and X₅₁. The amino acid residues in said positions are as defined above.

As discussed above, in some embodiments where the Fc binding polypeptides as disclosed herein form part of a three-helix bundle protein domain, they essentially form part of at least the three helices and the interconnecting loop between helix 1 and helix 2 (referred to as loop 1 (L1)) and the interconnecting loop between helix 2 and helix 3 (referred to as loop 1 (L2)). Herein, binding module Sequence C essentially forms part of at least the three helices and the interconnecting loop between helix 1 and helix 2 (referred to as loop 1 (L1)) and the interconnecting loop between helix 2 and helix 3 (referred to as loop 2 (L2)). It will be appreciated that the length of L1 and L2 may vary, for example by the addition or removal of one or more additional amino acid residues, provided that this variation does not significantly alter the three dimensional structure of polypeptide comprising binding module Sequence C.

As explained above, Sequence B comprises helix 1 (H1) and L1. The skilled person will appreciate that the amino acid sequences of H1 and L1 may be independently combined while maintaining the three-dimensional structure of the Fc binding polypeptide. Thus, in one embodiment, there is provided an Fc binding polypeptide as defined herein, which comprises a binding module Sequence C, which Sequence C consists of the sequences [H1], [L1] and [Sequence A] in the following order from the N-terminus to the C-terminus; [H1]-[L1]-[Sequence A], wherein [Sequence A] is as defined herein, and wherein, independently from each other, [H1] is selected from the group consisting of:

EQQNAFYEILH (SEQ ID NO:67); DQQAAFYEILH (SEQ ID NO:68); EAQEAFYEILH (SEQ ID NO:69); DQQSAFYEILH (SEQ ID NO:70); AQQAAFYEILH (SEQ ID NO:71); EQQNAFYEILN (SEQ ID NO:72); EQQAAFYEILH (SEQ ID NO:73); AQQSAFYEILH (SEQ ID NO:74); EQQQAFYEILH (SEQ ID NO:75); EHQNAFYEILH (SEQ ID NO:76); EAQNAFYEILH (SEQ ID NO:77); ETQNAFYEILH (SEQ ID NO:78); EQQSAFYEILH (SEQ ID NO:79); EAQNAFYKILH (SEQ ID NO:80); EQQEAFYEILH (SEQ ID NO:81); EAQKAFYEILK (SEQ ID NO:82); EQHNAFYEILH (SEQ ID NO:83); EHHHALYHILH (SEQ ID NO:84); EQQKAFYAILH (SEQ ID NO:85); DQQSAFYEILN (SEQ ID NO:86); AQQNAFYQVLN (SEQ ID NO:87); EQQKAFYEILH (SEQ ID NO:88); EQQKAFYEILK (SEQ ID NO:89); EQQNAFYEILS (SEQ ID NO:90); EQQNAFYHILH (SEQ ID NO:91); and EQQNAFYHILN (SEQ ID NO:92), and [L1] is selected from the group consisting of:
LPNLT (SEQ ID NO:93); LPNLN (SEQ ID NO:94); MPNLN (SEQ ID NO:95); LPYLT (SEQ ID NO:96); LPSLT (SEQ ID NO:97); MPNLL (SEQ ID NO:98); LPNGN (SEQ ID NO:99); LPNLS (SEQ ID NO:100); GPNAN (SEQ ID NO:101); VPNLN (SEQ ID NO:102); LPNLR (SEQ ID NO:103); and LPNLL (SEQ ID NO:104).

In one particular embodiment, H1 is selected from the group consisting of EQQNAFYEILH; DQQAAFYEILH; EAQEAFYEILH; DQQSAFYEILH; AQQAAFYEILH; EQQNAFYEILN; EQQAAFYEILH; AQQSAFYEILH; EQQQAFYEILH; and EHQNAFYEILH, such as the group consisting of EQQNAFYEILH; DQQAAFYEILH; EAQEAFYEILH; DQQSAFYEILH; AQQAAFYEILH; EQQNAFYEILN; and EQQAAFYEILH, such as the group consisting of EQQNAFYEILH; DQQAAFYEILH; EAQEAFYEILH; DQQSAFYEILH; and AQQAAFYEILH, such as the group consisting of EQQNAFYEILH; DQQAAFYEILH and EAQEAFYEILH. In one embodiment, H1 comprises or consists of EQQNAFYEILH. In one embodiment, H1 comprises or consists of DQQAAFYEILH. In one embodiment, H1 comprises or consists of EAQEAFYEILH.

In one particular embodiment, L1 is selected from the group consisting of LPNLT; LPNLN; MPNLN; LPYLT; LPSLT; MPNLL; LPNGN, and LPNLS, such as the group consisting of LPNLT; LPNLN; MPNLN; LPYLT; PSLT, and MPNLL; such as the group consisting of LPNLT; LPNLN; MPNLN and LPYLT; such as the group consisting of LPNLT; LPNLN and MPNLN; such as the group consisting of LPNLT; and LPNLN. In one embodiment, L1 comprises or consists of LPNLT. In one embodiment, L1 comprises or consists of LPNLN.

In one embodiment, there is provided an Fc binding polypeptide as disclosed herein, which comprises a binding module Sequence C selected from the group consisting of: EQQNAFYEILHLPNLT-[Sequence A](SEQ ID NO:105); EQQNAFYEILHLPNLN-[Sequence A](SEQ ID NO:106); DQQAAFYEILHLPNLT-[Sequence A](SEQ ID NO:107); EAQEAFYEILHLPNLT-[Sequence A](SEQ ID NO:108); DQQSAFYEILHLPNLT-[Sequence A](SEQ ID NO:109); AQQAAFYEILHLPNLT-[Sequence A](SEQ ID NO:110); EQQAAFYEILHLPNLT-[Sequence A](SEQ ID NO:111); AQQSAFYEILHLPNLT-[Sequence A](SEQ ID NO:112); EQQNAFYEILNMPNLN-[Sequence A](SEQ ID NO:113); EAQNAFYEILHLPNLT-[Sequence A](SEQ ID NO:114); EQQQAFYEILHLPNLT-[Sequence A](SEQ ID NO:115); EQQNAFYEILHLPYLT-[Sequence A](SEQ ID NO:116); ETQNAFYEILHLPNLT-[Sequence A](SEQ ID NO:117); EAQNAFYKILHLPNLT-[Sequence A](SEQ ID NO:118); EQQNAFYEILHLPNGN-[Sequence A](SEQ ID NO:119); EHQNAFYEILHLPNLN-[Sequence A](SEQ ID NO:120); EQQNAFYEILNMPNLL-[Sequence A](SEQ ID NO:121); EQQSAFYEILHLPNLT-[Sequence A](SEQ ID NO:122); EQQEAFYEILHLPNLT-[Sequence A](SEQ ID NO:123); EAQKAFYEILKLPNLT-[Sequence A](SEQ ID NO:124); EQHNAFYEILHLPNLN-[Sequence A](SEQ ID NO:125); EHHHALYHILHLPNLN-[Sequence A](SEQ ID NO:126); AQQSAFYEILHLPSLT-[Sequence A](SEQ ID NO:127); EQQKAFYAILHLPYLT-[Sequence A](SEQ ID NO:128); DQQSAFYEILNMPNLN-[Sequence A](SEQ ID NO:129); AQQNAFYQVLNMPNLN-[Sequence A](SEQ ID NO:130); EQQKAFYEILKLPNLT-[Sequence A](SEQ ID NO:131); EQQNAFYEILSLPNLT-[Sequence A](SEQ ID NO:132); EQQNAFYEILHLPNLS-[Sequence A](SEQ ID NO:133); EQQNAFYHILHLPNLN-[Sequence A](SEQ ID NO:134); EHQNAFYEILHLPNLT-[Sequence A](SEQ ID NO:135); EQQAAFYEILHLPNLN-[Sequence A](SEQ ID NO:136); AQQAAFYEILHLPSLT-[Sequence A](SEQ ID NO:137); wherein [Sequence A] is as defined herein.

In one particular embodiment, said binding module Sequence C is selected from the group consisting of: EQQNAFYEILHLPNLT-[Sequence A]; EQQNAFYEILHLPNLN-[Sequence A]; DQQAAFYEILHLPNLT-[Sequence A]; EAQEAFYEILHLPNLT-[Sequence A]; DQQSAFYEILHLPNLT-[Sequence A]; AQQAAFYEILHLPNLT-[Sequence A]; EQQAAFYEILHLPNLT-[Sequence A]; AQQSAFYEILHLPNLT-[Sequence A]; EQQNAFYEILNMPNLN-[Sequence A]; EAQNAFYEILHLPNLT-[Sequence A]; EQQQAFYEILHLPNLT-[Sequence A]; EQQNAFYEILHLPYLT-[Sequence A]; ETQNAFYEILHLPNLT-[Sequence A]; EAQNAFYKILHLPNLT-[Sequence A]; EQQNAFYEILHLPNGN-[Sequence A]; EHQNAFYEILHLPNLN-[Sequence A]; and EQQNAFYEILNMPNLL-[Sequence A]; such as the group consisting of: EQQNAFYEILHLPNLT-[Sequence A]; EQQNAFYEILHLPNLN-[Sequence A]; DQQAAFYEILHLPNLT-[Sequence A]; EAQEAFYEILHLPNLT-[Sequence A]; DQQSAFYEILHLPNLT-[Sequence A]; AQQAAFYEILHLPNLT-[Sequence A]; EQQAAFYEILHLPNLT-[Sequence A]; AQQSAFYEILHLPNLT-[Sequence A]; EQQNAFYEILNMPNLN-[Sequence A]; EAQNAFYEILHLPNLT-[Sequence A]; and EQQQAFYEILHLPNLT-[Sequence A]; such as the group consisting of: EQQNAFYEILHLPNLT-[Sequence A]; EQQNAFYEILHLPNLN-[Sequence A]; DQQAAFYEILHLPNLT-[Sequence A]; EAQEAFYEILHLPNLT-[Sequence A]; DQQSAFYEILHLPNLT-[Sequence A]; AQQAAFYEILHLPNLT-[Sequence A]; EQQAAFYEILHLPNLT-[Sequence A]; AQQSAFYEILHLPNLT-[Sequence A]; and EQQNAFYEILNMPNLN-[Sequence A]; such as the group consisting of: EQQNAFYEILHLPNLT-[Sequence A]; EQQNAFYEILHLPNLN-[Sequence A]; DQQAAFYEILHLPNLT-[Sequence A]; EAQEAFYEILHLPNLT-[Sequence A]; DQQSAFYEILHLPNLT-[Sequence A]; AQQAAFYEILHLPNLT-[Sequence A]; and EQQAAFYEILHLPNLT-[Sequence A]; such as the group consisting of: EQQNAFYEILHLPNLT-[Sequence A]; EQQNAFYEILHLPNLN-[Sequence A]; DQQAAFYEILHLPNLT-[Sequence A]; EAQEAFYEILHLPNLT-[Sequence A]; DQQSAFYEILHLPNLT-[Sequence A]; and AQQAAFYEILHLPNLT-[Sequence A]; such as the group consisting of: EQQNAFYEILHLPNLT-[Sequence A]; EQQNAFYEILHLPNLN-[Sequence A]; DQQAAFYEILHLPNLT-[Sequence A]; EAQEAFYEILHLPNLT-[Sequence A]; and DQQSAFYEILHLPNLT-[Sequence A]; such as the group consisting of: EQQNAFYEILHLPNLT-[Sequence A]; EQQNAFYEILHLPNLN-[Sequence A]; and EAQEAFYEILHLPNLT-[Sequence A]; such as the group consisting of: EQQNAFYEILHLPNLT-[Sequence A]; and EAQEAFYEILHLPNLT-[Sequence A].

In one particular embodiment, said binding module Sequence C comprises or consists of EQQNAFYEILHLPNLT-[Sequence A]. In one particular embodiment, said binding module Sequence C comprises or consists of EQQNAFYEILHLPNLN-[Sequence A]. In one particular embodiment, said binding module Sequence C comprises or consists of EAQEAFYEILHLPNLT-[Sequence A].

In another embodiment, there is provided an Fc binding polypeptide comprising a binding module Sequence C selected from the group consisting of:

vi)
(SEQ ID NO: 108)
EAQEAFYEILHLPNLT-[Sequence A]

wherein [Sequence A] is as defined herein;
vii) an amino acid sequence which has at least 85% identity to the sequence defined in vi);

viii)
(SEQ ID NO: 106)
EQQNAFYEILHLPNLN-[Sequence A]

wherein [Sequence A] is as defined herein; and
ix) an amino acid sequence which has at least 85% identity to the sequence defined in viii).

Also, in a further embodiment, there is provided an Fc binding polypeptide comprising a binding module Sequence C selected from the group consisting of:

vi)
(SEQ ID NO: 108)
EAQEAFYEILHLPNLT-[Sequence A]

wherein [Sequence A] is as defined herein; and
vii) an amino acid sequence which has at least 85% identity to the sequence defined in vi).

As discussed above, polypeptides comprising minor changes as compared to the above amino acid sequences without largely affecting the tertiary structure and the function thereof also fall within the scope of the present disclosure. Thus, in some embodiments, the Fc binding polypeptides comprising a binding module Sequence C as defined above may for example have a sequence which is at least 87%, such as at least 89%, such as at least 91%, such as at least 93%, such as at least 95%, such as at least 97% identical to a sequence defined by vi).

Also, in a further embodiment, there is provided an Fc binding polypeptide comprising a binding module Sequence C selected from the group consisting of:

viii)
(SEQ ID NO: 106)
EQQNAFYEILHLPNLN-[Sequence A]

wherein [Sequence A] is as defined herein; and
ix) an amino acid sequence which has at least 85% identity to the sequence defined in viii).

In some embodiments, the Fc binding polypeptides comprising a binding module Sequence C as defined above may for example have a sequence which is at least 87%, such as at least 89%, such as at least 91%, such as at least 93%, such as at least 95%, such as at least 97% identical to a sequence defined by viii).

As described in detail in the experimental section to follow, the selection of Fc binding polypeptide variants has led to the identification of a number of individual amino acid sequences and said sequences, also referred to herein as binding module Sequences C, constitute individual embodiments of sequence vi) or viii) corresponding to amino acid positions 8-54 in SEQ ID NO:1-57 and 208-264 presented in FIG. 10, as well as SEQ ID NO:269-305. Hence, the sequences of individual amino acid sequences according to Sequence C correspond to amino acid positions 8-54 in SEQ ID NO:1-57 and SEQ ID NO:208-264 presented in FIG. 10, as well as SEQ ID NO: 269-305. Hence, in one embodiment according to the second aspect as disclosed herein, there is provided an Fc binding polypeptide wherein sequence vi) or viii) corresponds to the sequence from position 8 to position 54 in a sequence selected from the group consisting of SEQ ID NO:1-57, 208-264 and 269-305, such as SEQ ID NO:1-56 and 208-263. In one embodiment, sequence vi) or viii) corresponds to the sequence from position 24 to position 54 in a sequence selected from the group consisting of SEQ ID NO:2-57, 209-264, and 269-305. In one embodiment, sequence vi) or viii) corresponds to the sequence from position 8 to position 54 in a sequence selected from the group consisting of SEQ ID NO:1-16 and 208-223 or the group consisting of SEQ ID NO:2-14, 16, 209-221 and 223. In one embodiment, sequence vi) or viii) corresponds to the sequence from position 24 to position 54 in a sequence selected from the group consisting of SEQ ID NO: SEQ ID NO:2-16 and 209-223. In one embodiment, sequence vi) or viii) corresponds to the sequence from position 8 to position 54 in a sequence selected from the group consisting of SEQ ID NO:2, 3, 4, 5, 6, 7, 8, 11, 12, 14, 209, 210, 211, 212, 213, 214, 215, 218, 219 and 221. In one embodiment, sequence vi) or viii) corresponds to the sequence from position 8 to position 54 in a sequence selected from the group consisting of SEQ ID NO:2, 4, 6, 7, 11, 12, 209, 211, 213, 214, 218 and 219. In one embodiment, sequence vi) or viii) corresponds to the sequence from position 8 to position 54 in a sequence selected from the group consisting of SEQ ID NO:6, 7, 11, 12, 213, 214, 218 and 219. In one embodiment, sequence vi) or viii) corresponds to the sequence from position 8 to position 54 in a sequence selected from the group consisting of SEQ ID NO:6, 7, 11, 213, 214 and 218. In one embodiment, sequence vi) or viii) corresponds to the sequence from position 8 to position 54 in a sequence selected from the group consisting of SEQ ID NO:7 and 214.

In one embodiment, there is provided an Fc binding polypeptide wherein sequence vi) corresponds to the sequence from position 8 to position 54 in a sequence selected from the group consisting of SEQ ID NO:1-57, such as SEQ ID NO:1-56. In one embodiment, sequence vi) corresponds to the sequence from position 8 to position 54 in a sequence selected from the group consisting of 2-57. In one embodiment, sequence vi) corresponds to the sequence from position 8 to position 54 in a sequence selected from the group consisting of SEQ ID NO:1-16 or the group consisting of SEQ ID NO:2-14 and 16. In one embodiment, sequence vi) corresponds to the sequence from position 8 to position 54 in a sequence selected from the group consisting of SEQ ID NO:2-16. In one embodiment, sequence vi) corresponds to the sequence from position 8 to position 54 in a sequence selected from the group consisting of SEQ ID NO:2, 3, 4, 5, 6, 7, 8, 11, 12 and 14. In one embodiment, sequence vi) corresponds to the sequence from position 8 to position 54 in a sequence selected from the group consisting of SEQ ID NO:2, 4, 6, 7, 11 and 12. In one embodiment, sequence vi) corresponds to the sequence from position 8 to position 54 in a sequence selected from the group consisting of SEQ ID NO:6, 7, 11 and 12. In one embodiment, sequence vi) corresponds to the sequence from position 8 to position 54 in a sequence selected from the group consisting of SEQ ID NO:6, 7 and 11. In one embodiment, sequence vi) corresponds to the sequence from position 8 to position 54 in SEQ ID NO:7.

In one embodiment according to the second aspect as disclosed herein, there is provided an Fc binding polypeptide wherein sequence viii) corresponds to the sequence from position 8 to position 54 in a sequence selected from the group consisting of SEQ ID NO:208-264 and 269-305, such as SEQ ID NO:208-263. In one embodiment, sequence viii) corresponds to the sequence from position 8 to position 54 in a sequence selected from the group consisting of SEQ ID NO:209-264 and 269-305. In one embodiment, sequence viii) corresponds to the sequence from position 8 to position 54 in a sequence selected from the group consisting of SEQ ID NO:208-223 or the group consisting of SEQ ID NO:209-221 and 223. In one embodiment, sequence viii) corresponds to the sequence from position 8 to position 54 in a sequence selected from the group consisting of SEQ ID NO:209-223. In one embodiment, sequence viii) corresponds to the sequence from position 8 to position 54 in a sequence selected from the group consisting of SEQ ID NO:209, 210, 211, 212, 213, 214, 215, 218, 219 and 221. In one embodiment, sequence viii) corresponds to the sequence from position 8 to position 54 in a sequence selected from the group consisting of SEQ ID NO:209, 211, 213, 214, 218 and 219. In one embodiment, sequence viii) corresponds to the sequence from position 8 to position 54 in a sequence selected from the group consisting of SEQ ID NO:213, 214, 218 and 219. In one embodiment, sequence viii) corresponds to the sequence from position 8 to position 54 in a sequence selected from the group consisting of SEQ ID NO:213, 214 and 218. In one embodiment, sequence viii) corresponds to the sequence from position 8 to position 54 in SEQ ID NO:214.

In some embodiments, the binding module Sequence C may form part of a polypeptide comprising an amino acid sequence further comprising Sequence 1 [S1] and/or Sequence 2 [S2]. In one embodiment, there is provided an Fc binding polypeptide as defined herein, comprising a Sequence 1 [S1], [binding module sequence C] and Sequence 2 [S2] in the following order from the N-terminus to the C-terminus

[S1]-[binding module sequence C]-[S2],
wherein [S1] or [S2] may be present or absent and [binding module sequence C] is as defined herein, and
wherein, independently from each other,
[S1] is selected from the group consisting of:

(SEQ ID NO: 138)
ADNKFNK;
(SEQ ID NO: 139)
VDAKFDK;
(SEQ ID NO: 140)
VDNKFNK;
(SEQ ID NO: 141)
IAAKHDK;
(SEQ ID NO: 142)
IAAQHDK;
(SEQ ID NO: 143)
ADNNFNK;
(SEQ ID NO: 144)
IAAKFDE;
(SEQ ID NO: 145)
PAAKHDK;
(SEQ ID NO: 146)
ADNAFNT;
FNK;
(SEQ ID NO: 147)
ADNRFNE;
(SEQ ID NO: 148)
IDSKFDE;
(SEQ ID NO: 149)
ADNRFNR;
(SEQ ID NO: 150)
ADNKHNK;
(SEQ ID NO: 151)
ADSKFDE;
(SEQ ID NO: 152)
IDAKHDE;
(SEQ ID NO: 153)
QQNKFNK;
(SEQ ID NO: 154)
ADNKFHK;
(SEQ ID NO: 155)
KFNK;
(SEQ ID NO: 156)
ADNNFNR;
(SEQ ID NO: 157)
AAAKHDK;
(SEQ ID NO: 158)
IDNKFNK;
(SEQ ID NO: 159)
IDAKFDE;
(SEQ ID NO: 160)
DNNFNK;
(SEQ ID NO: 161)
ADNKFNE;
(SEQ ID NO: 162)
AAAQHDK;
and
(SEQ ID NO: 163)
AAAKFDE;

and [S2] is selected from the group consisting of:

(SEQ ID NO: 164)
QAPK;
(SEQ ID NO: 165)
QAPP;
QAP;
(SEQ ID NO: 166)
QAPR;
(SEQ ID NO: 167)
QAPE;
APK;
(SEQ ID NO: 168)
QAPG;
(SEQ ID NO: 169)
QAIK;
and
QA.

In one embodiment, S1 is selected from the group consisting of ADNKFNK; VDAKFDK; VDNKFNK; IAAKHDK; IAAQHDK; ADNNFNK; IAAKFDE; PAAKHDK; ADNAFNT: FNK; ADNRFNE; and IDSKFDE, such as the group consisting of ADNKFNK; VDAKFDK; VDNKFNK; IAAKHDK; IAAQHDK; ADNNFNK; IAAKFDE; and PAAKHDK, such as the group consisting of ADNKFNK; VDAKFDK; VDNKFNK; IAAKHDK; IAAQHDK; and ADNNFNK, such as the group consisting of ADNKFNK; VDAKFDK; VDNKFNK; IAAKHDK; and IAAQHDK, such as the group consisting of ADNKFNK; VDAKFDK; VDNKFNK; and IAAKHDK, such as the group consisting of ADNKFNK; VDAKFDK; and VDNKFNK. In one embodiment, S1 comprises or consists of VDNKFNK. In one embodiment, S1 comprises or consists of VDAKFDK. In one embodiment, S1 comprises or consists of ADNKFNK.

In one embodiment, S2 is selected from the group consisting of QAPK; QAPP; QAP; QAPR; and QAPE, such as the group consisting of QAPK; QAPP; QAP; and QAPE, such as the group consisting of QAPK; QAPP; QAP; and QAPR, such as the group consisting of QAPK; QAPP and QAP; such as the group consisting of QAPK; and QAPP. In one embodiment, S2 comprises or consists of QAPK. In one embodiment, S2 comprises or consists of QAPP. In one embodiment, S2 comprises or consists of QAP.

In some embodiments, the Fc binding polypeptide may be selected from polypeptides of which Sequence A forms a part according to the following. Thus, the Fc binding polypeptide may comprise an amino acid sequence selected from the group consisting of:

(SEQ ID NO: 170)
ADNKFNKEQQNAFYEILHLPNLT-[Sequence A]-QAPK;
(SEQ ID NO: 171)
ADNKFNKEQQNAFYEILHLPNLN-[Sequence A]-QAPK;
(SEQ ID NO: 172)
VDAKFDKEAQEAFYEILHLPNLT-[Sequence A]-QAPK;
(SEQ ID NO: 173)
VDNKFNKEQQNAFYEILHLPNLN-[Sequence A]-QAPK;
(SEQ ID NO: 174)
VDNKFNKEQQNAFYEILHLPNLT-[Sequence A]-QAPK;
(SEQ ID NO: 175)
PAAKHDKDQQSAFYEILHLPNLT-[Sequence A]-QAPP;
(SEQ ID NO: 176)
ADNRFNREQQNAFYEILHLPNLT-[Sequence A]-QAPR;
(SEQ ID NO: 177)
VDAKFDKEAQNAFYEILHLPNLT-[Sequence A]-QAPK;
(SEQ ID NO: 178)
ADNAFNTEQQNAFYEILHLPNLT-[Sequence A]-QAPK;
(SEQ ID NO: 179)
VDAKFDKETQNAFYEILHLPNLT-[Sequence A]-QAPK;
(SEQ ID NO: 180)
ADNKFNKEQQNAFYEILHLPNGN-[Sequence A]-QAPKA;
(SEQ ID NO: 181)
ADNKFNKEHQNAFYEILHLPNLN-[Sequence A]-QAPK;
(SEQ ID NO: 182)
FNKEQQNAFYEILHLPNLN-[Sequence A]-QAPK;
(SEQ ID NO: 183)
VDAKFDKEQQNAFYEILHLPNLT-[Sequence A]-QAPK;
(SEQ ID NO: 184)
VDAKFDKEAQKAFYEILKLPNLT-[Sequence A]-QAPK;
(SEQ ID NO: 185)
ADNKFNKEQHNAFYEILHLPNLN-[Sequence A]-QAPK;
(SEQ ID NO: 186)
VDNKFNKEAQNAFYKILHLPNLT-[Sequence A]-QAPK;
(SEQ ID NO: 187)
VDAKFDKEQQEAFYEILHLPNLT-[Sequence A]-QAPK;
(SEQ ID NO: 188)
VDAKFDKEQQKAFYEILKLPNLT-[Sequence A]-QAPK;
(SEQ ID NO: 189)
ADNKFHKEQQNAFYEILHLPNLN-[Sequence A]-QAPK;
(SEQ ID NO: 190)
ADNKFNKEQQNAFYHILHLPNLN-[Sequence A]-QAPK;
(SEQ ID NO: 191)
ADNKFNKEHQNAFYEILHLPNLT-[Sequence A]-QAPK;
(SEQ ID NO: 192)
ADNKHNKEHHHALYHILHLPNLN-[Sequence A]-QAPK;
(SEQ ID NO: 193)
KFNKEQQNAFYEILHLPNLN-[Sequence A]-QAPK;
(SEQ ID NO: 194)
ADSKFDEAQQSAFYEILHLPSLT-[Sequence A]-QAPP;
and
(SEQ ID NO: 195)
IDAKFDEAQQAAFYEILHLPNLT-[Sequence A]-QAPP,
wherein
[Sequence A] is as defined herein.

In particular embodiments, the Fc binding polypeptide comprises an amino acid sequence selected from the group consisting of:

ADNKFNKEQQNAFYEILHLPNLN-[Sequence A]-QAPK;
VDAKFDKEAQEAFYEILHLPNLT-[Sequence A]-QAPK;
VDNKFNKEQQNAFYEILHLPNLN-[Sequence A]-QAPK;
VDNKFNKEQQNAFYEILHLPNLT-[Sequence A]-QAPK;
PAAKHDKDQQSAFYEILHLPNLT-[Sequence A]-QAPP;
ADNRFNREQQNAFYEILHLPNLT-[Sequence A]-QAPR;
VDAKFDKEAQNAFYEILHLPNLT-[Sequence A]-QAPK;
ADNAFNTEQQNAFYEILHLPNLT-[Sequence A]-QAPK,
and
VDAKFDKETQNAFYEILHLPNLT-[Sequence A]-QAPK;
ADNKFNKEQQNAFYEILHLPNLT-[Sequence A]-QAPK;

such as the group consisting of:

ADNKFNKEQQNAFYEILHLPNLT-[Sequence A]-QAPK;
ADNKFNKEQQNAFYEILHLPNLN-[Sequence A]-QAPK;
VDAKFDKEAQEAFYEILHLPNLT-[Sequence A]-QAPK;
VDNKFNKEQQNAFYEILHLPNLN-[Sequence A]-QAPK;
VDNKFNKEQQNAFYEILHLPNLT-[Sequence A]-QAPK;
PAAKHDKDQQSAFYEILHLPNLT-[Sequence A]-QAPP,
and
ADNRFNREQQNAFYEILHLPNLT-[Sequence A]-QAPR;

such as the group consisting of:

ADNKFNKEQQNAFYEILHLPNLT-[Sequence A]-QAPK;
ADNKFNKEQQNAFYEILHLPNLN-[Sequence A]-QAPK;
VDAKFDKEAQEAFYEILHLPNLT-[Sequence A]-QAPK;
VDNKFNKEQQNAFYEILHLPNLN-[Sequence A]-QAPK,
and
VDNKFNKEQQNAFYEILHLPNLT-[Sequence A]-QAPK;

such as the group consisting of:

ADNKFNKEQQNAFYEILHLPNLT-[Sequence A]-QAPK;
ADNKFNKEQQNAFYEILHLPNLN-[Sequence A]-QAPK;
and
VDAKFDKEAQEAFYEILHLPNLT-[Sequence A]-QAPK.

In one embodiment the Fc binding polypeptide comprises or consists of ADNKFNKEQQNAFYEILHLPNLT-[Sequence A]-QAPK. In one embodiment the Fc binding polypeptide comprises or consists of ADNKFNKEQQNAFYEILHLPNLN-[Sequence A]-QAPK. In one embodiment the Fc binding polypeptide comprises or consists of VDAKFDKEAQEAFYEILHLPNLT-[Sequence A]-QAPK.

In one embodiment, the Fc binding polypeptide comprises an amino acid sequence selected from:

x)
(SEQ ID NO: 172)
VDAKFDKE AQEAFYEILHLPNLT-[Sequence A]-QAPK

wherein [Sequence A] is as defined herein; and
xi) an amino acid sequence which has at least 86% identity to the sequence defined in x).

Again, polypeptides comprising minor changes as compared to the above amino acid sequences x) without largely affecting the tertiary structure and the function thereof are also within the scope of the present disclosure. Thus, in some embodiments, the Fc binding polypeptides as defined above may for example have a sequence which is at least 87%, such as at least 89%, such as at least 91%, such as at least 93%, such as at least 94%, such as at least 96%, such as at least 98% identical to the sequence defined by x).

Sequence x) in such a polypeptide may be selected from the group consisting of SEQ ID NO:1-57, such as SEQ ID NO:1-56. In one embodiment, sequence x) corresponds to a sequence selected from the group consisting of SEQ ID NO:2-57. In one embodiment, sequence x) corresponds to a sequence selected from the group consisting of SEQ ID NO:1-16 or the group consisting of SEQ ID NO:2-14 and 16. In one embodiment, sequence x) corresponds to a sequence selected from the group consisting of SEQ ID NO:2-16. In one embodiment, sequence x) corresponds to a sequence selected from the group consisting of SEQ ID NO:2, 3, 4, 5, 6, 7, 8, 11, 12 and 14. In one embodiment, sequence x) corresponds to a sequence selected from the group consisting of SEQ ID NO:2, 4, 6, 7, 11 and 12.

In one embodiment, sequence x) corresponds to a sequence selected from the group consisting of SEQ ID NO:6, 7, 11 and 12. In one embodiment, sequence x) corresponds to a sequence selected from the group consisting of SEQ ID NO:6, 7 and 11. In another embodiment, sequence x) corresponds to the sequence SEQ ID NO:7.

In one embodiment, the Fc binding polypeptide comprises an amino acid sequence selected from:

xii)
(SEQ ID NO: 170)
ADNKFNKEQQNAFYEILHLPNLT-[Sequence A]-QAPK

wherein [Sequence A] is as defined herein; and
xiii) an amino acid sequence which has at least 86% identity to the sequence defined in xii).

In yet another embodiment, the Fc binding polypeptide comprises an amino acid sequence selected from:

xiv)
(SEQ ID NO: 171)
ADNKFNKEQQNAFYEILHLPNLN-[Sequence A]-QAPK

wherein [Sequence A] is as defined herein; and
xv) an amino acid sequence which has at least 86% identity to the sequence defined in xiv).

In some embodiments, the Fc binding polypeptides as defined in xiii) or xv) may for example have a sequence which is at least 87%, such as at least 89%, such as at least 91%, such as at least 93%, such as at least 94%, such as at least 96%, such as at least 98% identical to the sequence defined by xii) or xiv).

In one embodiment, the Fc binding polypeptide comprises an amino acid sequence selected from:

xvi)
VDNKFNKEQQNAFYEILHLPNLN-[Sequence A]-QAPK

wherein [Sequence A] is as defined herein; and
xvii) an amino acid sequence which has at least 86% identity to the sequence defined in xvi).

In some embodiments, the Fc binding polypeptides as defined in xvii) may for example have a sequence which is at least 87%, such as at least 89%, such as at least 91%, such as at least 93%, such as at least 94%, such as at least 96%, such as at least 98% identical to the sequence defined by xvi).

Sequence xvi) in such a polypeptide may be selected from the group consisting of SEQ ID NO:208-264 and 269-305, such as SEQ ID NO:208-263. In one embodiment, sequence xvi) corresponds to a sequence selected from the group consisting of SEQ ID NO:209-264 and 269-305. In one embodiment, sequence xvi) corresponds to a sequence selected from the group consisting of SEQ ID NO:208-223 or the group consisting of SEQ ID NO:209-221 and 223. In one embodiment, sequence xvi) corresponds to a sequence selected from the group consisting of SEQ ID NO:209-223. In one embodiment, sequence xvi) corresponds to a sequence selected from the group consisting of SEQ ID NO:209, 210, 211, 212, 213, 214, 215, 218, 219 and 221. In one embodiment, sequence xvi) corresponds to a sequence selected from the group consisting of SEQ ID NO:209, 211, 213, 214, 218 and 219.

In one embodiment, sequence xvi) corresponds to a sequence selected from the group consisting of SEQ ID NO:213, 214, 218 and 219. In one embodiment, sequence xvi) corresponds to a sequence selected from the group consisting of SEQ ID NO:213, 214 and 218. In another embodiment, sequence xvi) corresponds to the sequence SEQ ID NO:214.

In one embodiment, the Fc binding polypeptide comprises an amino acid sequence selected from

x)
VDAKFDKE AQEAFYEILHLPNLT-[Sequence A]-QAPK

wherein [Sequence A] is as defined herein;
xi) an amino acid sequence which has at least 86% identity to the sequence defined in x);

xvi)
VDNKFNKEQQNAFYEILHLPNLN-[Sequence A]-QAPK

wherein [Sequence A] is as defined herein; and
xvii) an amino acid sequence which has at least 86% identity to the sequence defined in xvi).

In some embodiments, the Fc binding polypeptides as defined in xi) or xvii) may for example have a sequence which is at least 87%, such as at least 89%, such as at least 91%, such as at least 93%, such as at least 94%, such as at least 96%, such as at least 98% identical to the sequence defined by x) or xvi).

In one embodiment according to the second aspect as disclosed herein, there is provided an Fc binding polypeptide wherein sequence x) or xvi) corresponds to a sequence selected from the group consisting of SEQ ID NO:1-57 and 208-264 and 269-305, such as SEQ ID NO:1-56 and 208-263. In one embodiment, sequence x) or xvi) corresponds to a sequence selected from the group consisting of SEQ ID NO:2-57 and 209-264 and 269-305. In one embodiment, sequence x) or xvi) corresponds to a sequence selected from the group consisting of SEQ ID NO:1-16 and 208-223 or the group consisting of SEQ ID NO:2-14, 16, 209-221 and 223. In one embodiment, sequence x) or xvi) corresponds to a sequence selected from the group consisting of SEQ ID NO:2-16 and 209-223. In one embodiment, sequence x) or xvi) corresponds to a sequence selected from the group consisting of SEQ ID NO:2, 3, 4, 5, 6, 7, 8, 11, 12, 14, 209, 210, 211, 212, 213, 214, 215, 218, 219 and 221. In one embodiment, sequence x) or xvi) corresponds to a sequence selected from the group consisting of SEQ ID NO:2, 4, 6, 7, 11, 12, 209, 211, 213, 214, 218 and 219. In one embodiment, sequence x) or xvi) corresponds to a sequence selected from the group consisting of SEQ ID NO:6, 7, 11, 12, 213, 214, 218 and 219. In one embodiment, sequence x) or xvi) corresponds to a sequence selected from the group consisting of SEQ ID NO:6, 7, 11, 213, 214 and 218. In one embodiment, sequence vi) or viii) corresponds to a sequence selected from the group consisting of SEQ ID NO:7 and 214.

A further polypeptide domain with the same functionality may be attached to the Fc binding polypeptide as defined herein, for example any Fc binding polypeptide as defined in i) to xvii), thus achieving a Fc binding polypeptide multimer. Such multimers may in particular be useful to increase the efficiency of binding the Fc region, for example to ensure that more Fc region containing polypeptides are bound to the Fc binding polypeptide multimer compared to the monomer variants thereof (in other words the Fc binding polypeptides). Thus, in a third aspect of the present disclosure, there is provided an Fc binding polypeptide multimer. Said multimer is understood to comprise at least two Fc binding polypeptides as disclosed herein as monomer units, the amino acid sequences of which may be the same or different. Thus, in one embodiment, there is provided an Fc binding polypeptide multimer wherein each monomer of the multimer comprises a Sequence A which is independently selected from any Sequence A defined herein. In another embodiment, there is provided an Fc binding polypeptide multimer wherein each monomer of the multimer comprises a Fc binding polypeptide which is independently selected from any Fc binding polypeptide defined herein.

Multimeric forms of the polypeptides may comprise a suitable number of domains, each having an Fc binding activity, and each forming a monomer within the multimer. In one embodiment, said multimer is selected from the group consisting of dimer, trimer, tetramer, pentamer, hexamer, heptamer, octamer, nonamer and decamer, such as selected from the group consisting of tetramer, pentamer, hexamer, heptamer and octamer; such as the group consisting of pentamer, hexamer and heptamer. In one particular embodiment, said multimer is a hexamer.

These monomers within the multimer may have the same amino acid sequence, but alternatively, they may have different amino acid sequences. Thus the monomers may have any amino acid sequence of the Fc binding polypeptides as defined herein, for example all monomers within the multimer may have different sequences or only a subset of the monomers in the multimer may have different amino acid sequences while other have the same sequence. In other words, the Fc binding polypeptide of the invention may form homo- or heteromultimers, for example homo- or heterodimers. In one embodiment, there is provided an Fc binding polypeptide multimer, wherein the multimer is a homomer. In another embodiment, there is provided an Fc binding polypeptide multimer, wherein multimer is a heteromer. Said heteromer may comprise at least two or at least three different Sequence A, wherein each Sequence A is as defined herein. In one embodiment, said multimer is a heteromer comprising at least two or at least three different Fc binding polypeptides, each as defined herein.

In one particular embodiment, said multimer is a homomer comprising at least three, such as at least four, such as at least five, such as at least six, such as at least seven, such as at least eight monomers selected from any one of SEQ ID NO:1-57 and 208-264 and 269-305, such as any one of SEQ ID NO:1-56 and 208-263, such as any one of SEQ ID NO:1-16 and SEQ ID NO:208-223, such as any one of SEQ ID NO:2-14, 16, 209-221 and 223, such as any one of SEQ ID NO:2, 3, 4, 5, 6, 7, 8, 11, 12, 14, 209, 210, 211, 212, 213, 214, 215, 218, 219 and 221; such as any one of SEQ ID NO:2, 4, 6, 7, 12, 209, 211, 213, 214 and 219, such as any one of SEQ ID NO:6, 7, 12, 213, 214 and 219; such as any one of SEQ ID NO:6, 7, 213 and 214; such as any one of SEQ ID NO:7 and SEQ ID NO:214.

In one particular embodiment, said multimer is a homomer comprising at least three, such as at least four, such as at least five, such as at least six, such as at least seven, such as at least eight monomers selected from any one of SEQ ID NO:208-264 and 269-305, such as any one of SEQ ID NO: 208-263, such as any one of SEQ ID NO:208-223, such as any one of SEQ ID NO: 209-221 and 223, such as any one of SEQ ID NO: 209, 210, 211, 212, 213, 214, 215, 218, 219 and 221; such as any one of SEQ ID NO:209, 211, 213, 214 and 219, such as any one of SEQ ID NO: 213, 214 and 219; such as any one of SEQ ID NO:213 and 214; such as of SEQ ID NO:214.

In one particular embodiment, said multimer is a homomer comprising at least three, such as at least four, such as at least five, such as at least six, such as at least seven, such as at least eight monomers selected from any one of SEQ ID NO:1-57, such as any one of SEQ ID NO:1-56, such as any one of SEQ ID NO:1-16, such as any one of SEQ ID NO:2-14 and 16, such as any one of SEQ ID NO:2, 3, 4, 5, 6, 7, 8, 11, 12 and 14; such as any one of SEQ ID NO:2, 4, 6, 7 and 12, such as any one of SEQ ID NO:6, 7 and 12; such as any one of SEQ ID NO:6 and 7; such as of SEQ ID NO:7.

In one particular embodiment, said multimer is a homomer comprising six monomers of SEQ ID NO:7, in other words said multimer is a homohexamer of SEQ ID NO:7. In another embodiment, the multimer is a homohexamer of SEQ ID NO:6. In one embodiment, the multimer is a homohexamer of SEQ ID NO:12. In one embodiment, said multimer is a homomer comprising six monomers of SEQ ID NO:214, in other words said multimer is a homohexamer of SEQ ID NO:214. In another embodiment, the multimer is a homohexamer of SEQ ID NO:213. In one embodiment, the multimer is a homohexamer of SEQ ID 219.

In particular embodiments, the multimer comprises a sequence selected from the group consisting of SEQ ID NO:203, 204 and 205. In one embodiment, said multimer comprises the sequence SEQ ID NO: 204.

It will be appreciated that monomers in the multimers discussed herein, which multimers comprise Sequence A or comprise Fc binding polypeptides as disclosed herein, may be directly coupled to each other or spaced apart by linker sequences. The skilled person appreciates that the presence or absence of linker sequences may be different between different monomers moieties in a multimer, and if linkers are present, the sequence of each individual linker may be the same or different.

As the skilled person understands, the construction of a multimer, for example as a fusion protein, often involves use of linkers between the monomer moieties to be fused. The skilled person is aware of different kinds of linkers with different properties, such as flexible amino acid linkers, rigid amino acid linkers and cleavable amino acid linkers. Linkers may be used in order to for example increase stability or improve folding of fusion proteins, to increase expression or to improve activity, affinity and/or binding capacity, Thus, in one embodiment, the Fc binding polypeptide multimer as defined herein further comprises at least one linker. In one embodiment, a linker is present between each monomer within the multimer. The linker may for example be selected from the group consisting of flexible amino acid linkers, rigid amino acid linkers and cleavable amino acid linkers. Alternatively, the linker may be a non-peptidic linker. Thus, the Fc binding polypeptides disclosed herein may be linked to each directly by peptide bonds between the C-terminal and N-terminal ends of the polypeptides. Alternatively, two or more monomers, in other words monomer units or moieties, within the multimer can be linked by elements comprising oligomeric or polymeric species, such as elements comprising up to 15 or 30 amino acids, such as 1-5, 1-10 or 5-10 amino acids. The nature of such a link should preferably not destabilize the spatial conformation of the protein units, that is of the Fc binding polypeptide monomers within the multimer. This can e.g. be achieved by avoiding the presence of proline in the linkers. Furthermore, said linkers should preferably also be sufficiently stable in alkaline environments not to impair the properties of the mutated protein units. For this purpose, it is advantageous if the linkers do not contain asparagine. It can additionally be advantageous if the linker do not contain glutamine. The multimer may further at the N-terminal end comprise a plurality of amino acid residues originating from the cloning process or constituting a residue from a cleaved off signaling sequence. The number of additional amino acid residues may e.g. be 15 or less, such as 10 or less or 5 or less.

The skilled person will understand that various modifications and/or additions can be made to an Fc binding polypeptide or to an Fc binding polypeptide multimer according to any aspect disclosed herein in order to tailor the polypeptide or multimer to a specific application without departing from the scope of the present disclosure.

For example, in one embodiment there is provided an Fc binding polypeptide or an Fc binding polypeptide multimer as described herein, which polypeptide has been extended by and/or comprises additional amino acids at the C terminus and/or N terminus. Such a polypeptide or multimer should be understood as a polypeptide or multimer having one or more additional amino acid residues at the very first and/or the very last position in the polypeptide chain, i.e. at the N- and/or C-terminus of the polypeptide or multimer. For example said additional amino acid residues may be at the N- and/or C-terminus any one of Sequence A according to i), ii) or iii), binding module Sequence C according to vi), vii), viii) or ix) or sequence x), xi), xii), xiii), xiv), xv) xvi) or xvii). Thus, the Fc binding polypeptides as defined herein may comprise any suitable number of additional amino acid residues, for example one, two, three, four, five, six, seven, eight, nine, ten or more additional amino acid residues. Said amino acid residues may individually or collectively improve production, purification, stabilization in vitro or coupling of the polypeptide to substrates of interest, for example to a solid support, such as a solid support described in connection to the aspect of a separation matrix.

Said additional amino residues may be coupled to the Fc binding polypeptide or multimer by means of chemical conjugation (using known organic chemistry methods) or by any other means, such as expression of the Fc binding polypeptide or multimer as a fusion protein or joined in any other fashion, either directly or via a linker, for example an amino acid linker as described above.

In some embodiments, the Fc binding polypeptides and/or multimer, as disclosed above, further comprises at the C-terminal or N-terminal end one or more coupling elements, selected from the group consisting of a cysteine residue, a plurality of lysine residues and a plurality of histidine residues. The coupling element may e.g. be a single cysteine at the C-terminal end. The coupling element(s) may be directly linked to the C- or N-terminal end, or it/they may be linked via a linker comprising up to 15 amino acids, such as 1-5, 1-10 or 5-10 amino acids. This stretch should preferably also be sufficiently stable in alkaline environments not to impair the properties of the mutated protein. For this purpose, it is advantageous if the stretch does not contain asparagine. It can additionally be advantageous if the stretch does not contain glutamine. An advantage of having a C-terminal cysteine is that endpoint coupling of the protein can be achieved through reaction of the cysteine thiol with an electrophilic group on a support. This provides excellent mobility of the coupled protein which is important for the binding capacity.

In a fourth aspect of the present disclosure, there is provided a polynucleotide encoding an Fc binding polypeptide or an Fc binding polypeptide multimer as described herein; an expression vector comprising said polynucleotide; and a host cell comprising said expression vector.

Also encompassed by this disclosure is a method of producing the Fc binding polypeptide or the Fc binding polypeptide multimer as described herein, comprising culturing said host cell under conditions permissive of expression of said polypeptide or multimer from its expression vector, and isolating the polypeptide or multimer.

The Fc binding polypeptide or multimer as disclosed herein may alternatively be produced by non-biological peptide synthesis using amino acids and/or amino acid derivatives having protected reactive side-chains, the non-biological peptide synthesis comprising
step-wise coupling of the amino acids and/or the amino acid derivatives to form a polypeptide according to the second aspect or a multimer according to the third aspect having protected reactive side-chains,
removal of the protecting groups from the reactive side-chains of the polypeptide or multimer, and
folding of the polypeptide or multimer in aqueous solution.

The terms “Fc binding” and “binding affinity for Fc” as used in this disclosure refer to a property of a polypeptide which may be tested for example by ELISA or the use of surface plasmon resonance (SPR) technology.

Fc binding affinity may be tested in an experiment in which Fc, or a fragment thereof, is immobilized on a sensor chip of the surface plasmon resonance (SPR) instrument, and the sample containing the polypeptide to be tested is passed over the chip. Alternatively, the polypeptide to be tested is immobilized on a sensor chip of the instrument, and a sample containing Fc, or a fragment thereof, is passed over the chip. The skilled person may then interpret the results obtained by such experiments to establish at least a qualitative measure of the binding affinity of the polypeptide for Fc. If a quantitative measure is desired, for example to determine a K_{D} value for the interaction, surface plasmon resonance methods may also be used. Binding values may for example be defined in a Biacore (Cytiva) or ProteOn XPR 36 (Bio-Rad) instrument. Fc is suitably immobilized on a sensor chip of the instrument, and samples of the polypeptide whose affinity is to be determined are prepared by serial dilution and injected in random order. K_{D} values may then be calculated from the results using for example the 1:1 Langmuir binding model of the BIAevaluation 4.1 software, or other suitable software, provided by the instrument manufacturer.

As explained above the Fc binding polypeptide or Fc binding polypeptide multimer of the present disclosure are able to bind to the Fc region of either IgG1, IgG2 or IgG4 with said affinity.

Thus in one embodiment, there is provided an Fc binding polypeptide or Fc binding polypeptide multimer as disclosed herein, which is capable of binding to Fc such as that the K_{D} value of the interaction is at most 1×10⁻⁷ M, such as at most 1×10⁻⁸ M, such as at most 1×10⁻⁹ M, such as at most 1×10⁻¹⁰ M, such as at most 1×10⁻¹¹ M.

It will be appreciated that the Fc binding polypeptides and/or the Fc binding polypeptide multimers exhibit a similar K_{D} value of the interaction with Fc as SEQ ID NO:58 and/or SEQ ID NO:59. In particular, the K_{D} value of the interaction with Fc of the Fc binding polypeptide or Fc binding polypeptide multimer as disclosed herein may differ with one order of magnitude from the K_{D} value of the interaction of SEQ ID NO:58 and/or SEQ ID NO:59 with Fc. For example, if the K_{D} value of the interaction of SEQ ID NO:58 and/or SEQ ID NO:59 with a particular Fc is 9×10⁻¹⁰ M in an assay, then the K_{D} value of the interaction with Fc of the Fc binding polypeptide or Fc binding polypeptide multimer may be in the range of from 9×10⁻⁹ M to 9×10⁻¹¹ M (one order of magnitude), in that assay. The skilled person will appreciate that it may be useful to compare K_{D} values obtained using the same assay and while some variation may occur between different assays, intra-assay comparisons generally demonstrate the same trends independent of the assay employed. Assays may for example differ depending on if it is the Fc containing polypeptide or the Fc binding polypeptide which is immobilized on a chip. In one embodiment, said K_{D} value of the interaction with Fc is measured in an assay wherein the Fc binding polypeptide is immobilized on the chip. In a different embodiment, the K_{D} value of the interaction with Fc is measured in an assay wherein the Fc containing polypeptide is immobilized on the chip.

In another embodiment, there is provided an Fc binding polypeptide or Fc binding polypeptide multimer as disclosed herein, which is capable of binding to Fc such as that the K_{D} value of the interaction is at least 50%, such as at least 60%, such as at least 70%, such as at least 80%, such as at least 90% of the K_{D} value of the interaction of SEQ ID NO:58 with Fc. In one embodiment, said Fc binding polypeptide or Fc binding polypeptide multimer is capable of binding to Fc such as that the K_{D} value of the interaction is at least 50%, such as at least 60%, such as at least 70%, such as at least 80%, such as at least 90% of the K_{D} value of the interaction of SEQ ID NO:59 with Fc. In one embodiment, said Fc binding polypeptide or Fc binding polypeptide multimer is capable of binding to Fc such as that the K_{D} value of the interaction is at least 50%, such as at least 60%, such as at least 70%, such as at least 80%, such as at least 90% of the K_{D} value of the interaction of SEQ ID NO:58 and SEQ ID NO:59 with Fc.

Similarly, the terms “VH3 binding” and “binding affinity for VH3” as used in this disclosure refer to a property of a polypeptide which may be tested for example by ELISA or the use of surface plasmon resonance (SPR) technology. VH3 binding affinity may be tested in an experiment in which VH3, or a fragment thereof, is immobilized on a sensor chip of the surface plasmon resonance (SPR) instrument, and the sample containing the polypeptide to be tested is passed over the chip. Alternatively, the polypeptide to be tested (in this case the Fc binding polypeptide) may be immobilized on a sensor chip of the instrument, and a sample containing VH3, or a fragment thereof, is passed over the chip. The skilled person appreciates that essentially an absence of binding is to be detected if the binding to VH3 is abolished or significantly reduced. The skilled person may then interpret the results obtained by such experiments to establish at least a qualitative measure of the binding affinity of the polypeptide for VH3. If a quantitative measure is desired, for example to determine a K_{D} value for the interaction, surface plasmon resonance methods may also be used in an analogous manner to what is explained above of Fc binding.

As explained in connection with the second aspect of the present disclosure, the presently identified Fc binding polypeptides and Fc binding polypeptide multimers as defined herein have significantly reduced or abolished binding affinity for VH3 of trastuzumab. The skilled person will appreciate that the present Fc binding polypeptides and Fc binding polypeptide multimers do not significantly interact with VH3, and thus their capacity to bind to VH3 is low.

In one embodiment, there is provided an Fc binding polypeptide or Fc binding polypeptide multimer as disclosed herein, which is not capable of binding to VH3 with a K_{D} value of the interaction of less than 1×10⁻⁴ M, such as less than 1×10⁻³ M.

In addition, the Fc binding polypeptide displays improved alkali stability. In embodiments where the Fc binding polypeptide is used for separation or isolation of e.g. antibodies/immunoglobulins having an Fc region, high alkali stability will allow for use of highly alkaline conditions during cleaning, essential for long-term repeated use in a bioprocess separation setting.

In one embodiment, there is provided an Fc binding polypeptide or Fc binding polypeptide multimer as disclosed herein which has an alkali stability of at least 60%, such as at least 70%, such as at least 80%, such as at least 85%, such as at least 89%, such as at least 90%, such as at least 95%, such as at least 98%, such as at least 99% of the alkali stability of SEQ ID NO:58 after incubation in 0.5 M NaOH. In one embodiment, said Fc binding polypeptide or Fc binding polypeptide multimer has an alkali stability of at least 60%, such as at least 70%, such as at least 80%, such as at least 85%, such as at least 89%, such as at least 90%, such as at least 95%, such as at least 98%, such as at least 99% of the alkali stability of SEQ ID NO:59 after incubation in 0.5 M NaOH. In one embodiment, said Fc binding polypeptide or Fc binding polypeptide multimer has an alkali stability of at least 60%, such as at least 70%, such as at least 80%, such as at least 85%, such as at least 89%, such as at least 90%, such as at least 95%, such as at least 98%, such as at least 99% of the alkali stability of SEQ ID NO:58 and SEQ ID NO:59 after incubation in 0.5 M NaOH. In one embodiment, said incubation is at least for 12 hours, such as at least for 24 hours or more. Said incubation may be for example at 22+/−2° C.

In another aspect, the present invention discloses a separation matrix, comprising an Fc binding polypeptide according to any embodiment disclosed above coupled to a solid support. Such a matrix is useful for separation of immunoglobulins or other Fc-containing proteins from e.g. a sample. Since the Fc binding polypeptide has binding affinity to Fc but low or no binding affinity to VH3, the separation matrix may allow for efficient separation of Fc containing proteins from any VH3 containing proteins or impurities. As described above, the Fc binding polypeptide displays improved alkali stability which will allow for use of highly alkaline conditions during cleaning of the separation matrix, essential for long-term repeated use in a bioprocess separation setting. The alkali stability of the matrix can be assessed by measuring the immunoglobulin-binding capacity, or Fc binding capacity, typically using polyclonal human IgG, before and after incubation in alkaline solutions at a specified temperature, e.g. 22+/−2° C. The incubation can e.g. be performed in 0.5 M or 1.0 M NaOH for a number of 15 min cycles, such as 100, 200 or 300 cycles, corresponding to a total incubation time of 25, 50 or 75 h. The IgG capacity of the matrix after 96-100 15 min incubation cycles or a total incubation time of 24 or 25 h in 0.5 M NaOH at 22+/−2° C. can be at least 80, such as at least 85, at least 90 or at least 95% of the IgG capacity before the incubation. The capacity of the matrix after a total incubation time of 24 h in 1.0 M NaOH at 22+/−2° C. can be at least 70, such as at least 80 or at least 90% of the IgG capacity before the incubation. In one embodiment, the separation matrix comprises an Fc binding polypeptide multimer, wherein each monomer comprises an Fc binding polypeptide as defined herein. The multimer, which is a homo-multimer or a hetero-multimer, may be a dimer, trimer, tetramer, pentamer, hexamer, heptamer, octamer, nonamer or decamer. For example, the separation matrix may comprise a hexamer of an Fc binding polypeptide coupled to the solid support. Each monomer of the Fc binding polypeptide multimer may comprise a Sequence A which is independently selected from any Sequence A defined herein. In particular, it may comprise a sequence selected from the group consisting of SEQ ID NO:1-57, 208-264 and 269-305, such as SEQ ID NO:1-56 and 208-263, such as SEQ ID NO:1-16 and SEQ ID NO:208-223, such as SEQ ID NO:2-14, 16, 209-221 and 223, such as SEQ ID NO:2, 3, 4, 5, 6, 7, 8, 11, 12, 14, 209, 210, 211, 212, 213, 214, 215, 218, 219 and 221; such as SEQ ID NO:2, 4, 6, 7, 12, 209, 211, 213, 214 and 219, such as SEQ ID NO:6, 7, 12, 213, 214, and 219; such as SEQ ID NO:6, 7, 213 and 214; such as SEQ ID NO:7 and SEQ ID NO:214. It may comprise a sequence selected from the group consisting of SEQ ID NO:1-57, such as SEQ ID NO:1-56. In particular, it may comprise a sequence selected from the group consisting of SEQ ID NO:1-16; such as from the group consisting of SEQ ID NO:2-14 and 16, such as from the group consisting of SEQ ID NO:2, 3, 4, 5, 6, 7, 8, 11, 12 and 14; such as from the group consisting of SEQ ID NO:2, 4, 6, 7 and 12, such as from the group consisting of SEQ ID NO:6, 7 and 12; such as from the group consisting of SEQ ID NO:6 and 7, such as SEQ ID NO:7. In one embodiment, said separation matrix comprises an Fc binding polypeptide multimer, optionally a hexamer, wherein each monomer of said multimer comprises a sequence corresponding to SEQ ID NO:7. It may comprise a sequence selected from the group consisting of SEQ ID NO:208-264 and 269-305, such as from the group consisting of SEQ ID NO:208-263, such as from the group consisting of SEQ ID NO:208-223, such as from the group consisting of SEQ ID NO:209-221 and 223, such as from the group consisting of SEQ ID NO:209, 210, 211, 212, 213, 214, 215, 218, 219 and 221; such as from the group consisting of SEQ ID NO:209, 211, 213, 214 and 219, such as from the group consisting of SEQ ID NO:213, 214 and 219; such as from the group consisting of SEQ ID NO:213 and 214. In one embodiment, said separation matrix comprises an Fc binding polypeptide multimer, optionally a hexamer, wherein each monomer of said multimer comprises a sequence corresponding to SEQ ID NO:214.

As the skilled person will understand, the expressed Fc binding polypeptide, in monomeric or multimeric form, should be purified to an appropriate extent before being immobilized to a support. Such purification methods are well known in the field, and the immobilization of protein-based ligands to supports is easily carried out using standard methods. Suitable methods and supports will be discussed below in more detail and are disclosed e.g. in WO16079033 which is incorporated herein by reference.

The solid support of the matrix according to the invention can be of any suitable well-known kind. As a non-limiting example, a conventional affinity separation matrix is often of organic nature and based on polymers that expose a hydrophilic surface to the aqueous media used, i.e. expose hydroxy (—OH), carboxy (—COOH), carboxamido (—CONH2, possibly in N-substituted forms), amino (—NH2, possibly in substituted form), oligo- or polyethylenoxy groups on their external and, if present, also on internal surfaces. The solid support can suitably be porous. The porosity can be expressed as a Kav or Kd value (the fraction of the pore volume available to a probe molecule of a particular size) measured by inverse size exclusion chromatography, e.g. according to the methods described in Gel Filtration Principles and Methods, Pharmacia LKB Biotechnology 1991, pp 6-13. By definition, both Kd and Kav values always lie within the range 0-1. The Kav value can advantageously be 0.6-0.95, e.g. 0.7-0.90 or 0.6-0.8, as measured with dextran of Mw 110 kDa as a probe molecule. An advantage of this is that the support has a large fraction of pores able to accommodate both the polypeptides/multimers of the invention and immunoglobulins binding to the polypeptides/multimers and to provide mass transport of the immunoglobulins to and from the binding sites.

The polypeptides or multimers may be attached to the support via conventional coupling techniques utilizing e.g. thiol, amino and/or carboxy groups present in the ligand. Bisepoxides, epichlorohydrin, CNBr, N-hydroxysuccinimide (NHS) etc. are well-known coupling reagents. Between the support and the polypeptide/multimer, a molecule known as a spacer can be introduced, which improves the availability of the polypeptide/multimer and facilitates the chemical coupling of the polypeptide/multimer to the support. Depending on the nature of the polypeptide/multimer and the coupling conditions, the coupling may be a multipoint coupling (e.g. via a plurality of lysines) or a single point coupling (e.g. via a single cysteine).

Alternatively, the polypeptide/multimer may be attached to the support by non-covalent bonding, such as physical adsorption or biospecific adsorption.

In some embodiments the matrix comprises 5-25, such as 5-20 mg/ml, 5-15 mg/ml, 5-11 mg/ml or 6-11 mg/ml of the polypeptide or multimer coupled to the support. The amount of coupled polypeptide/multimer can be controlled by the concentration of polypeptide/multimer used in the coupling process, by the activation and coupling conditions used and/or by the pore structure of the support used. As a general rule the absolute binding capacity of the matrix increases with the amount of coupled polypeptide/multimer, at least up to a point where the pores become significantly constricted by the coupled polypeptide/multimer. The relative binding capacity per mg coupled polypeptide/multimer will decrease at high coupling levels, resulting in a cost-benefit optimum within the ranges specified above.

In certain embodiments the polypeptides or multimers are coupled to the support via thioether bonds. Methods for performing such coupling are well-known in this field and easily performed by the skilled person in this field using standard techniques and equipment. Thioether bonds are flexible and stable and generally suited for use in affinity chromatography. In particular when the thioether bond is via a terminal or near-terminal cysteine residue on the polypeptide or multimer, the mobility of the coupled polypeptide/multimer is enhanced which provides improved binding capacity and binding kinetics. In some embodiments the polypeptide/multimer is coupled via a C-terminal cysteine provided on the protein as described above. This allows for efficient coupling of the cysteine thiol to electrophilic groups, e.g. epoxide groups, halohydrin groups etc. on a support, resulting in a thioether bridge coupling.

In certain embodiments the solid support comprises a polyhydroxy polymer, such as a polysaccharide. Examples of polysaccharides include e.g. dextran, starch, cellulose, pullulan, agar, agarose etc. Polysaccharides are inherently hydrophilic with low degrees of nonspecific interactions, they provide a high content of reactive (activatable) hydroxyl groups and they are generally stable towards alkaline cleaning solutions used in bioprocessing. In some embodiments the support comprises agar or agarose. The supports used in the present invention can easily be prepared according to standard methods, such as inverse suspension gelation (S Hjerten: Biochim Biophys Acta 79(2), 393-398 (1964)). Alternatively, the base matrices are commercially available products, such as crosslinked agarose beads sold under the name of SEPHAROSE™ FF (Cytiva). In an embodiment, which is especially advantageous for large-scale separations, the support has been adapted to increase its rigidity using the methods described in U.S. Pat. No. 6,602,990 or U.S. Pat. No. 7,396,467, which are hereby incorporated by reference in their entirety, and hence renders the matrix more suitable for high flow rates.

In certain embodiments the support, such as a polysaccharide or agarose support, is crosslinked, such as with hydroxyalkyl ether crosslinks. Crosslinker reagents producing such crosslinks can be e.g. epihalohydrins like epichlorohydrin, diepoxides like butanediol diglycidyl ether, allylating reagents like allyl halides or allyl glycidyl ether. Crosslinking is beneficial for the rigidity of the support and improves the chemical stability. Hydroxyalkyl ether crosslinks are alkali stable and do not cause significant nonspecific adsorption.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L'Industria 70(9), 70-75 (1988)). As an alternative, a commercially available product, such as SOURCE™ (Cytiva) may be used. Alternatively, the solid support according to the invention comprises a support of inorganic nature, e.g. silica, zirconium oxide etc.

In one embodiment, the solid support has the form of a porous monolith. In an alternative embodiment, the support is in beaded or particle form that can be porous or non-porous. Solid supports in beaded or particle form can be used as a packed bed or in a suspended form. Suspended forms include those known as expanded beds and pure suspensions, in which the particles or beads are free to move. In case of monoliths, packed bed and expanded beds, the separation procedure commonly follows conventional chromatography with a concentration gradient. In case of pure suspension, batch-wise mode will be used.

In one embodiment, the separation matrix is a convection-based chromatography matrix. Such convection-based chromatography matrix may comprise a fibrous support or fibrous substrate. Said fibrous support may be based on electrospun polymeric fibers or cellulose fibers, optionally non-woven fibers. The fibrous support may thus be a fibrous non-woven polymer matrix. The fibers comprised in said fibrous support have a cross-sectional diameter of 10-1000 nm, such as 200-800 nm, 200-400 nm or 300-400 nm. Such a fibrous support can be found in a HiTrap Fibro™ unit (Cytiva). Alternative fibrous supports are disclosed in e.g. WO2019/137869 and WO2018/011600.

In yet another embodiment, the solid support is in another form such as a surface, a chip, capillaries, or a filter (e.g. a membrane or a depth filter matrix).

In another aspect, the present invention provides a method of isolating an immunoglobulin, wherein a separation matrix as disclosed above is used. In some embodiments, the method comprises a) contacting a liquid sample comprising an immunoglobulin with a separation matrix as disclosed above. The method may furthermore comprise b) washing said separation matrix with a washing liquid, c) eluting the immunoglobulin from the separation matrix with an elution liquid, and optionally d) cleaning the separation matrix with a cleaning liquid. The cleaning liquid can alternatively be called a cleaning-in-place (CIP) liquid. The contact (incubation) time may be at least 10 min.

The method may also comprise steps of, before step a), providing an affinity separation matrix according to any of the embodiments described above and providing a solution comprising an immunoglobulin and at least one other substance as a liquid sample and of, after step c), recovering the eluate and optionally subjecting the eluate to further separation steps, e.g. by anion or cation exchange chromatography, multimodal chromatography and/or hydrophobic interaction chromatography. Suitable compositions of the liquid sample, the washing liquid and the elution liquid, as well as the general conditions for performing the separation are well known in the art of affinity chromatography and in particular in the art of Protein A chromatography. The liquid sample comprising an Fc-containing protein and at least one other substance may comprise host cell proteins (HCP), such as CHO cell, E. Coli or yeast proteins. Contents of CHO cell and E. Coli proteins can conveniently be determined by immunoassays directed towards these proteins, e.g. the CHO HCP or E. Coli HCP ELISA kits from Cygnus Technologies. The host cell proteins or CHO cell/E. Coli proteins may be desorbed during step b). The elution may be performed by using any suitable solution used for elution from Protein A media. This can e.g. be a solution or buffer with pH 5 or lower, such as pH 2.5-5 or 3-5. It can also in some cases be a solution or buffer with pH 11 or higher, such as pH 11-14 or pH 11-13. In some embodiments the elution buffer or the elution buffer gradient comprises at least one mono- di- or trifunctional carboxylic acid or salt of such a carboxylic acid. In certain embodiments the elution buffer or the elution buffer gradient comprises at least one anion species selected from the group consisting of acetate, citrate, glycine, succinate, phosphate, and formiate.

In some embodiments, the cleaning liquid is alkaline, such as with a pH of 13-14. Such solutions provide efficient cleaning of the matrix, in particular at the upper end of the interval.

In certain embodiments, the cleaning liquid comprises 0.1-2.0 M NaOH or KOH, such as 0.5-2.0 or 0.5-1.0 M NaOH or KOH. These are efficient cleaning solutions, and in particular so when the NaOH or KOH concentration is above 0.1 M or at least 0.5 M. The high stability of the polypeptides of the invention enables the use of such strongly alkaline solutions.

The method may also include a step of sanitizing the matrix with a sanitization liquid, which may e.g. comprise a peroxide, such as hydrogen peroxide and/or a peracid, such as peracetic acid or performic acid.

In some embodiments, steps a)-d) are repeated at least 10 times, such as at least 50 times, 50-200, 50-300 or 50-500 times. This is important for the process economy in that the matrix can be re-used many times. Steps a)-c) can also be repeated at least 10 times, such as at least 50 times, 50-200, 50-300 or 50-500 times, with step d) being performed after a plurality of instances of step c), such that step d) is performed at least 10 times, such as at least 50 times. Step d) can e.g. be performed every second to twentieth instance of step c).

The terms an “Fc binding polypeptide” and “Fc binding protein” mean a polypeptide or protein respectively, capable of binding to the crystallizable part (Fc) of an antibody and includes e.g. Protein A, or any fragment or fusion protein thereof that has maintained said binding property.

The term “linker” herein means an element linking two polypeptide units, monomers or domains to each other in a multimer.

The term “spacer” herein means an element connecting a polypeptide or a polypeptide multimer to a support.

The term “VH binding” refers to binding to the variable heavy (VH) chain of an antibody, or the so-called Fab fragment. The term “Fab fragment” and “VH chain” may be used interchangeably herein, and subsequently the term “Fab binding” is used interchangeably with the term “VH binding”.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1a is an overview of Biacore set-up for screening of library of Fc binding polypeptide variants.

FIG. 1b shows sensorgrams of Fab and Fc-binding of albumin binding domain-linked domain Z (SEQ ID NO:59-ABD) and an inventive Fc binding polypeptide linked to ABD (candidate-ABD).

FIG. 2 is an example plot of alkali stability for tested Fc binding polypeptide variants in comparison to SEQ ID NO:58.

FIG. 3 shows Biacore binding curves (reference-subtracted) following injection of 1 g/L trastuzumab Fab.

FIG. 4 shows Biacore binding curves (reference-subtracted) following injection of 40 mg/L Fc.

FIG. 5 shows Biacore binding curves (reference-subtracted) following injection of 50 mg/L trastuzumab.

FIG. 6 shows Biacore binding curves (reference-subtracted) following injection of 0.5 g/L control antibody which does not contain the VH3 of trastuzumab

FIG. 7 shows Biacore binding capacity as a function of ligand coupling level for four different target proteins.

FIG. 8 is a plot of trastuzumab response as a function of number of NaOH injections for hexameric Fc binding polypeptide ligands.

FIG. 9 is a Plot of trastuzumab response as a function of number of NaOH injections for monomeric Fc binding polypeptide ligands.

FIG. 10 is a is a listing of the amino acid sequences of examples of Fc binding polypeptides of the present disclosure (SEQ ID NO:1-57 and SEQ ID NO:208-264) and hexamer variants thereof (SEQ ID NO:203-205), as well amino acid sequences of domain A, B, C, D and E of Staphylococcal protein A and derivatives thereof (SEQ ID NO:58-64). In the Fc binding polypeptides of the present disclosure, the deduced Sequence A extend from residue 24 to residue 54 in each sequence. The amino acid sequences Sequence B extend from residue 8-23 and binding module Sequence C predicted to constitute the complete three-helix bundle within each of these Fc binding polypeptides extend from residue 8 to residue 54.

FIG. 11 shows Biacore binding curves (reference-subtracted) for full antibodies trastuzumab and adalimumab. 11a) Domain B_{wt} (SEQ ID NO 61) and trastuzumab; 11b) Domain B_{wt} (SEQ ID NO 61) and adalimumab; 11c) mutated domain B (SEQ ID 275) and trastuzumab; 11d) mutated domain B (SEQ ID 275) and adalimumab.

FIG. 12 shows Biacore binding curves (reference-subtracted) for Fc fragments from adalimumab and for Fab fragments from trastuzumab. 12a) Domain B_{wt} (SEQ ID NO 61) and Fc fragment; 12b) Domain B_{wt} (SEQ ID NO 61) and Fab fragment; 12c) mutated domain B (SEQ ID 275) and Fc fragment; 12d) mutated domain B (SEQ ID 275) and Fab fragment.

### EXAMPLES

### Summary of Examples 1 to 3

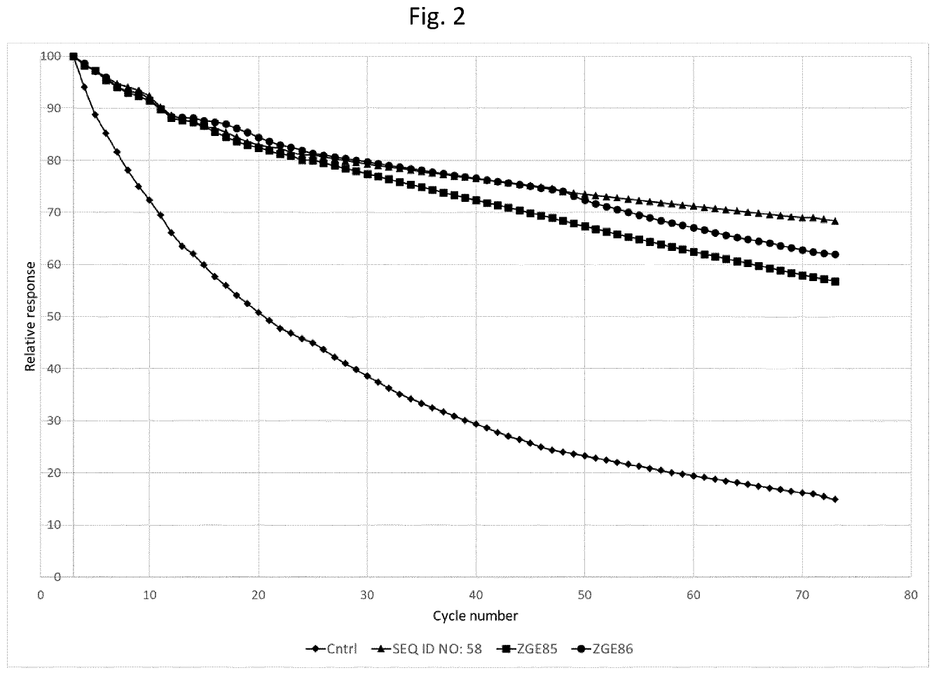

This project aimed for providing Fc binding polypeptides with reduced or abolished ability to bind VH3 compared to SEQ ID NO:59 or 58. Three positions (33, 40 and 51) in SEQ ID NO:58 were identified as key positions to knock out VH binding and randomized in a Fc binding polypeptide variant library. The library was screened for the following parameters: no VH binding, high Fc binding and high alkaline stability. The set-up for analysis of the members of the library is outlined in FIG. 2.

From the screening, a subset of ligands were chosen for further studies on column. The further studies included multimerization of the ligands to hexamers and expression of said hexamers in E. coli. On these hexameric Fc binding polypeptide ligands, a primary characterization was performed, wherein the ligands were evaluated for dynamic binding capacity (DBC), pH elution, no binding of Fab and alkaline stability.

Trastuzumab used in the Examples below was produced/purified from in-house production cell line (CHO-K1 background).

### Example 1

### Biacore Evaluation of Fc Binding Polypeptide VH Knock-Out Library and Sequencing of Candidates

The impact of mutations at key amino acid positions was investigated to identify positions that enabled reduced binding to the VH3 region. The three positions with the highest impact on VH3 (in a Fab fragment) binding were identified as amino acid positions 33/40/51 of SEQ ID NO:58.

To identify optimal Fc binding polypeptide candidates—a small library with a diversity of amino acids at the three identified positions (33/40/51) were designed and constructed (Table 1). The total diversity of the library was 648 possible variants.

TABLE 1
Summary of library randomization.
Library randomization

| Position 33 | Position 40 | Position 51 |
|---|---|---|
| P | H | L |
| T | Q | I |
| A | N | V |
| H | K | R |
| Q | D | S |
| N | E | G |
| K | R | |
| D | S | |
| E | G | |
| R | | |
| S | | |
| G | | |

### Generation of Candidate Lysates and Colony Storage Plates

For evaluation of the mutant library all the Fc binding polypeptide variants were constructed as fusion proteins with C-terminal fusion to the Albumin Binding Domain (ABD) (SEQ ID NO:201). This allowed for efficient screening of variants from raw E. coli lysates via use of capture to immobilized RSA (Rat Serum Albumin; high affinity for ABD). In short, the generation of protein was achieved by transformation of KCM competent E. coli top10 with the library in plasmid form. Colonies representing individual variants were generated by plating the transformation product on agar plates, followed by incubation at 37° C. overnight for individual colonies to appear.

Individual colonies were transferred from the agar plates to 96-well Deep Well Plates (96DWP, 10 in total) for culture and protein production. Pipette tips used to pipette up/down 10-times to ensure bacterial dispersion and then 3-5 μl from each well was transferred to a marked agar plate in marked 96-grid structure for later retrieval of interesting candidates for sequencing, re-culture or re-cloning (colony storage plate).

The 96DWPs were incubated in an Infors HT shaking incubator for 24 hours at 27° C. and 300 rpm shaking. Finished cultures were frozen at −20° C.

To generate crude protein containing lysates; frozen 96DWPs were incubated in 80° C. water bath for 2 hours followed by pelleting of cell debris by centrifugation at 3400×g for 40 min. These crude lysate plates with the raw protein were then kept in cooler until further analysis.

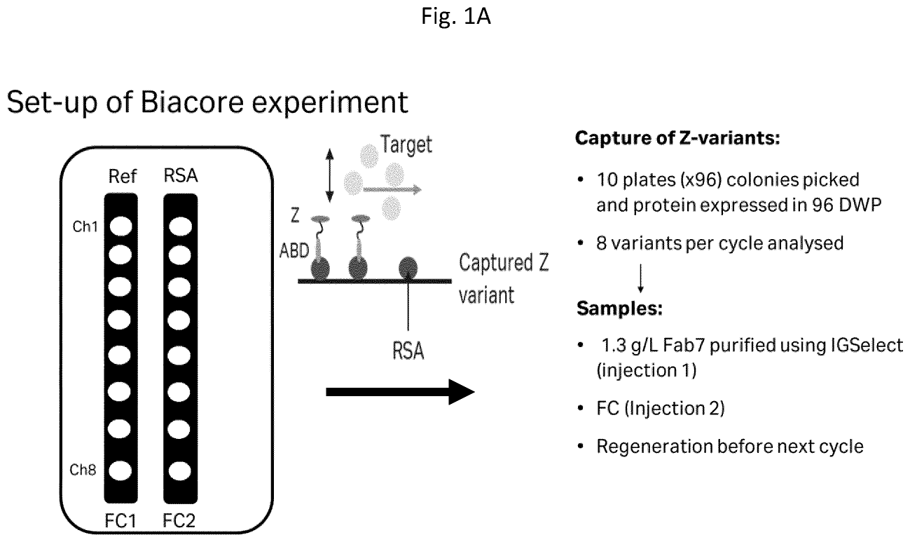

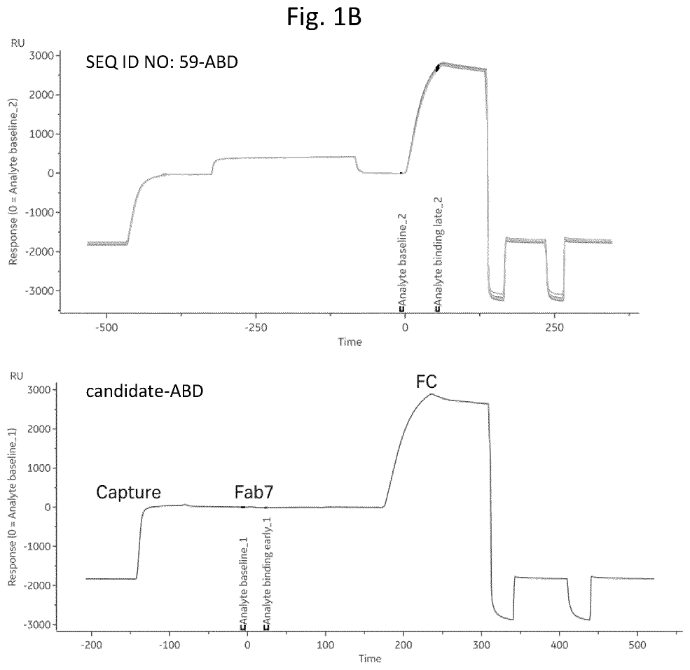

Raw protein lysates were generated by heat treatment (2 hours at 80° C.) in Biacore running buffer. Variants were then screened for expression level (capture to immobilized RSA), binding to Fab prepared from trastuzumab (SEQ ID NO:198 and 199; Fab fragment of trastuzumab, comprising VH3) and binding to Fc (SEQ ID NO:200) from trastuzumab. FIG. 1 shows an overview of a Biacore set-up.

### Pre-Testing of Antibody Fragment Material

An analysis of the antibody fragment material Fab (comprising SEQ ID NO:198 and 199) and Fc (SEQ ID NO:200) was performed using albumin binding domain-linked domain Z (Z-ABD) (SEQ ID NO:202).

### Materials and Methods

Z-ABD (SEQ ID NO:202), Fab fragment of trastuzumab (comprising SEQ ID NO:198 and 199) and Fc (SEQ ID NO:200) were obtained from preparations generated in-house. Biacore CM5 chips; RSA; Biacore EHS coupling kit; Biacore 8K+ instrument (all from Cytiva, Uppsala, Sweden) were used according to manufacturer's instructions. RSA was immobilized on all channels of FC2 on a CM5 chip (50 ug/ml RSA pH5 in acetate buffer). The immobilized amount was ranging between 13500-14000 Ru. FC1 was blocked with ethanolamine.

To test binding to available Fab and Fc, an analysis run using Z-ABD was performed. Z-ABD was diluted 1:1 with PBS-P+ (phosphate buffered saline with KCl and Tween20), Fab fragment of trastuzumab (also referred to as Fab below) was diluted to 1.3 g/L in pBS-P+ (Prep 1), and Fc was diluted to 18 μg/ml in PBS-P+. Z was captured on FC2, followed by injection of Fab and then Fc. The resulting sensorgram is shown in FIG. 1B (a).

### Biacore Analysis of Fc Binding Polypeptide VH Knock-Out Candidates

### Materials and Methods

CM5 sensor chip with RSA was immobilized on all channels of FC2 (50 ug/ml RSA pH5 in acetate buffer). Biacore 8K+ instrument (Cytiva) was used for analysis, E. coli lysates of the VH knock-out candidate Fc binding polypeptides were prepared as described above. Fab fragment of trastuzumab as used at the concentration of 1.3 g/L in pBS-P+ and the Fc preparation used was 18 μg/ml in PBS-P+).

Two methods for the Biacore analysis were used as described below:

### Biacore Method 1:

Biacore method 1 setup was as follows:
Running buffer: PBS-P+
Flow-rate: 5 μl/min during analysis; 30 μl/min during regeneration
Injections: 60 s capture of candicate Fc binding polypeptide fused to ABD (SEQ ID NO:201) (1+2 dilution in PBS-P+), 240 s injection of 1.3 g/L Fab fragment of trastuzumab preparation 1 diluted in PBS-P+, 60 s injection of 18 μg/ml Fc diluted in PBS-P+ (all injections over both flow cells).
Regeneration: 2×30 s injections with Glycin-HCl pH 1.5.

### Biacore Method 2:

Biacore method 2 setup was identical to above except a reduction of injection time for Fab fragment of trastuzumab to preserve sample and use of a new Fab fragment of trastuzumab batch (preparation 2). New injection time was 90 s. The resulting sensorgram of a candidate Fc binding polypeptide according to present invention is shown in FIG. 1B. The candidate binds to VH3 with a lower affinity than SEQ ID NO:59, while maintaining affinity for Fc.

### Selection of Top Candidate Fc Binding Polypeptides

Based on screening results, candidates were selected for identification by sequencing using the following criteria:
(1) All candidates giving a Fab response below zero were taken forward.
(2) Of these, all candidates giving a capture level above 1600 Ru taken forward.
(3) Remaining candidates sorted based on Fc to Capture level ratio*.
(4) Top 70 candidates based on Fc to capture level selected for sequencing.

*It should be noted that both Fab and Fc interaction was impacted by the amount of candidate captured in the first step. Due to this, selection of candidates based on retained Fc interaction was done using the ratio of the Fc signal and the Capture level (referred to as Fc to Capture).

Selected candidates had a Fc to capture ratio of 1.35-1.68. The data for Z (SEQ ID NO:59) above gives a Fc to capture ratio of around 1.55-1.6 in comparison.

### Sequencing of Selected Fc Binding Polypeptide VH Knock-Out Candidates

### Materials and Methods

For sequencing the material used was: colony storage plates, Agar-MTP plate (Eurofins), and B069 sequencing primer (IDT). Selected colonies were transferred from the colony storage plates to Agar-MTP plates using pipette tips. Agar-MTP plates were sent together with the B069 primer to Eurofins for sequencing. Sequencing results analyzed using Geneious prime software.

### Results

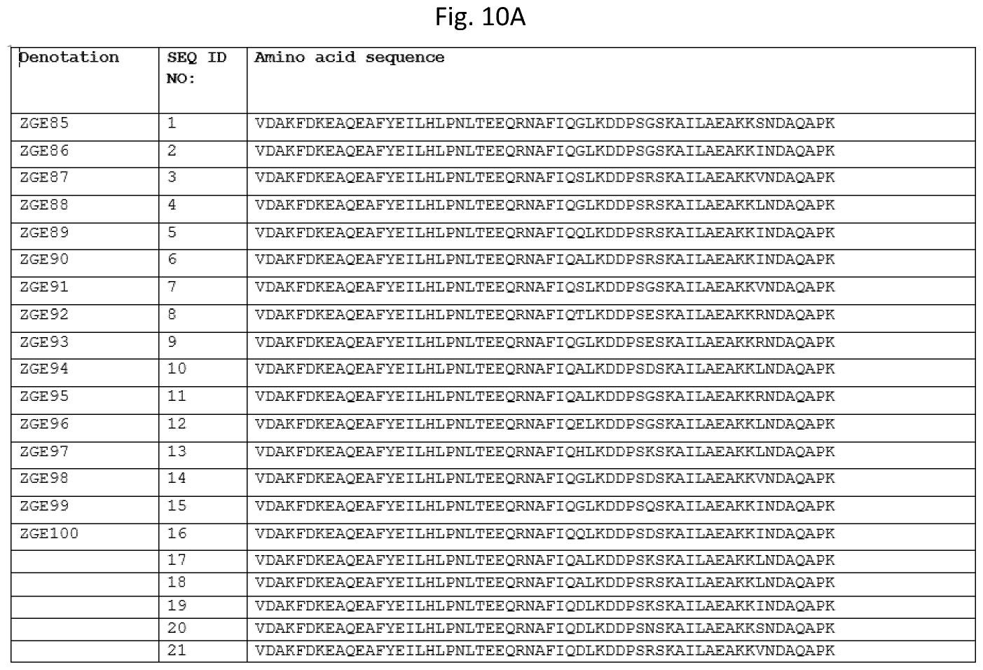

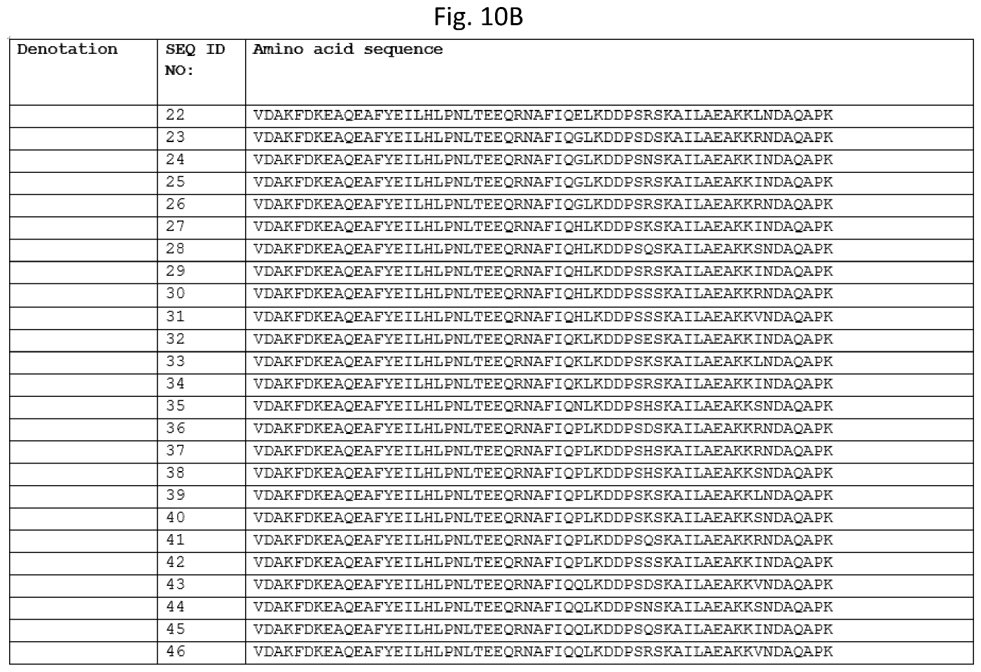

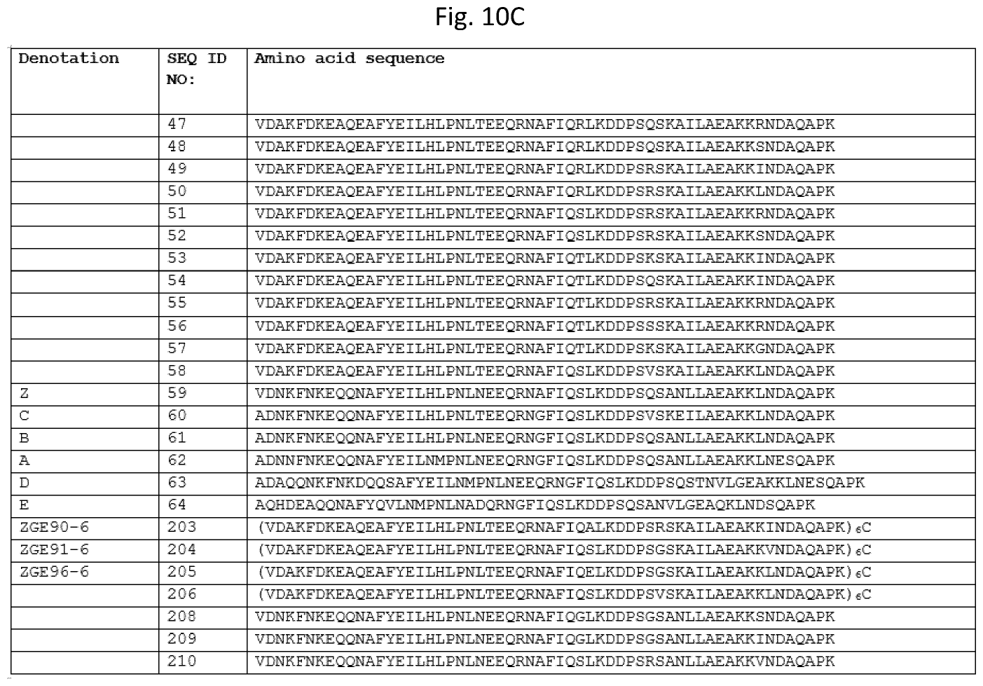

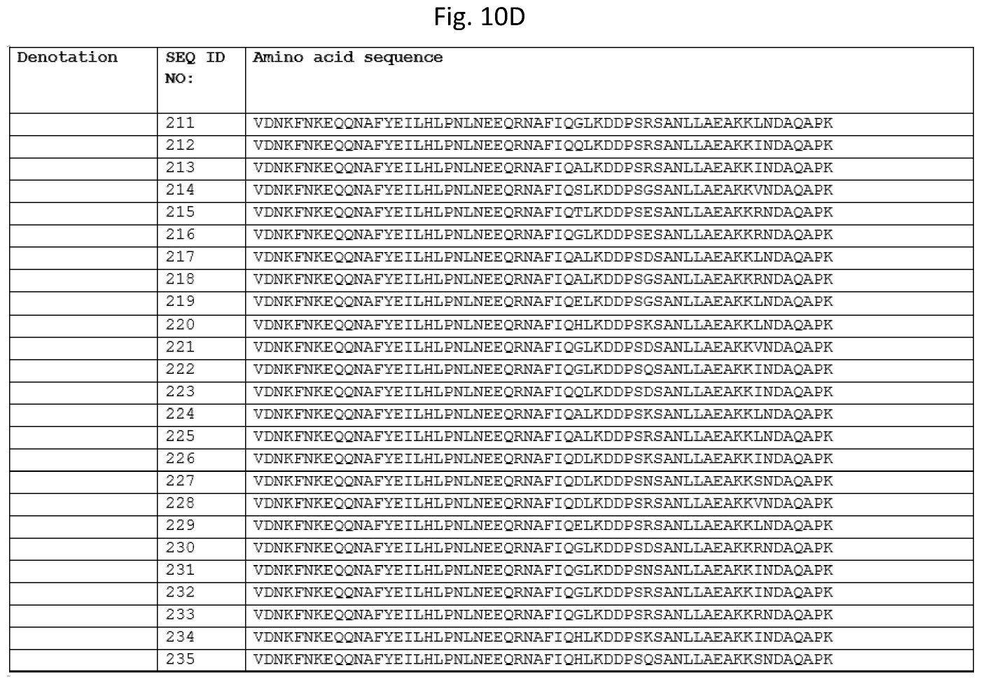

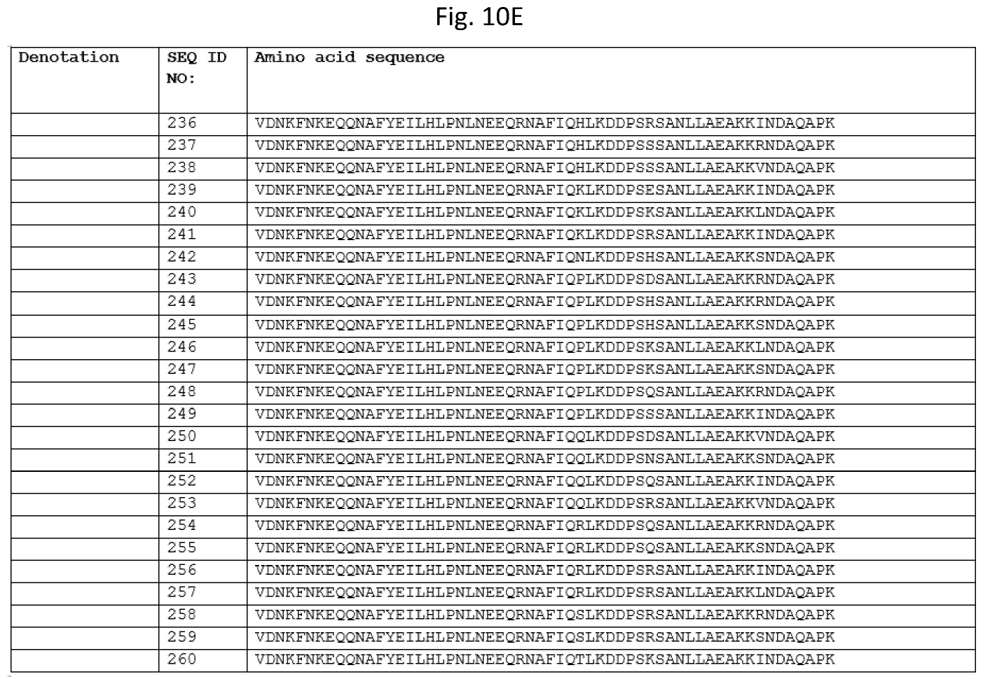

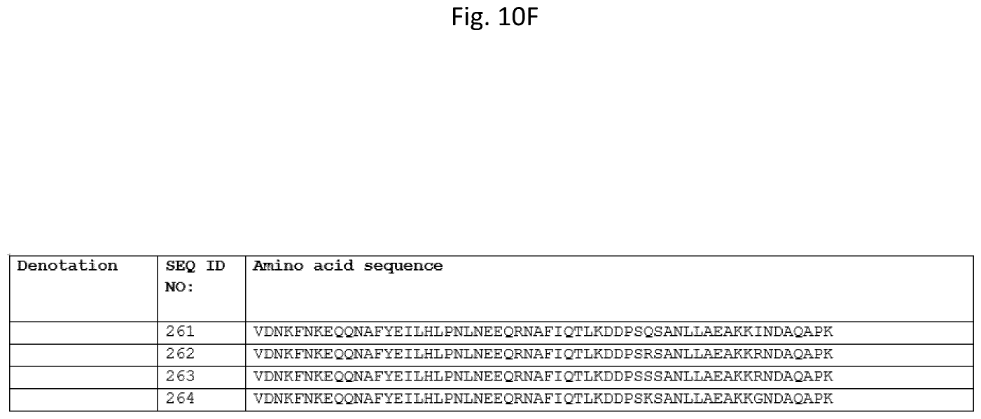

57 unique Fc binding polypeptides were obtained corresponding to SEQ ID NO:1-57 in FIG. 10. Table 2 shows Biacore data and sequencing results for these candidates. The table is sorted based on Fc to Capture level ratio. Sequence alterations outside targeted region occurred for some variants and are indicated in comments. The most frequently occurring amino acid residues in positions 33, 40 and 51 are summarized in Table 3.

TABLE 2
Biacore data and sequencing results for the 57 candidates.

| | Analyte | Analyte | FC to | Seq variant |
|---|---|---|---|---|
| Capture | binding | binding | Capture | positions |
| level_1 | early_1: Fab | late_2: FC | ratio | 33-40-51 |
| 1762 | −37.6 | 2873 | 1.63 | ADL |
| 1787 | −42.4 | 2914 | 1.63 | AGR |
| 1821 | −17.2 | 2925 | 1.61 | AKL |
| 1784 | −18.9 | 2907 | 1.63 | ARI |
| 2084 | −2.85 | 3114 | 1.49 | ARL |
| 1790 | −18.8 | 2881 | 1.61 | DK |
| 1741 | −35.1 | 2860 | 1.64 | DNS |
| 1762 | −17.9 | 2832 | 1.61 | DRV |
| 1713 | −32.6 | 2849 | 1.66 | EGL |
| 1772 | −18.3 | 2845 | 1.60 | ERL |
| 1736 | −41.1 | 2832 | 1.63 | GDR |
| 1738 | −19.4 | 2900 | 1.67 | GDV |
| 1704 | −40.8 | 2862 | 1.68 | GER |
| 1782 | −20.3 | 2875 | 1.61 | GGI |
| 2867 | −8.61 | 4363 | 1.52 | GGS |
| 1704 | −33.8 | 2836 | 1.66 | GNI |
| 1774 | 83.6 | 2965 | 1.67 | GQI |
| 2073 | −4.06 | 3224 | 1.56 | GRI |
| 1775 | −19.0 | 2927 | 1.65 | GRL |
| 2126 | −3.11 | 3249 | 1.53 | GRR |
| 2099 | −4.41 | 3117 | 1.48 | HKI |
| 1773 | −37.3 | 2913 | 1.64 | HKL |
| 1740 | −28.8 | 2845 | 1.64 | HQS |
| 1801 | −17.9 | 2907 | 1.61 | HRI |
| 1813 | −16.5 | 2934 | 1.62 | HSR |
| 1818 | −15.7 | 2950 | 1.62 | HSV |
| 1818 | −18.5 | 2916 | 1.60 | KE |
| 2180 | −8.37 | 2972 | 1.36 | KKL |
| 1810 | −34.0 | 2969 | 1.64 | KRI |
| 1763 | −40.6 | 2876 | 1.63 | NHS |
| 1793 | −17.3 | 2923 | 1.63 | PDR |
| 1785 | −40.9 | 2909 | 1.63 | PHR |
| 1730 | −40.9 | 2861 | 1.65 | PHS |
| 1757 | −35.4 | 2891 | 1.65 | PKL |
| 1780 | −17.3 | 2879 | 1.62 | PKS |
| 1729 | −33.3 | 2839 | 1.64 | PQR |
| 1728 | −33.0 | 2847 | 1.65 | PSI |
| 1755 | −34.6 | 2891 | 1.65 | QDI |
| 1753 | −17.6 | 2857 | 1.63 | QDV |
| 1774 | −20.9 | 2861 | 1.61 | QNS |
| 1729 | −17.1 | 2811 | 1.63 | QQI |
| 1787 | −19.1 | 2895 | 1.62 | QRI |
| 1806 | −19.0 | 2901 | 1.61 | QRV |
| 1679 | −33.8 | 2797 | 1.67 | RQR |
| 1786 | −17.0 | 2869 | 1.61 | RQS |
| 2247 | −8.19 | 3026 | 1.35 | RRI |
| 1705 | −39.3 | 2818 | 1.65 | RRL |
| 1801 | −18.7 | 2927 | 1.63 | SGV |
| 1817 | −15.8 | 2927 | 1.61 | SRR |
| 1816 | −18.8 | 2929 | 1.61 | SRS |
| 1856 | −19.3 | 3051 | 1.64 | SRV |
| 1739 | −22.6 | 2916 | 1.68 | TER |
| 1746 | −15.6 | 2864 | 1.64 | TKG |
| 1743 | −18.5 | 2843 | 1.63 | TKI |
| 1753 | −21.2 | 2813 | 1.61 | TQI |
| 1714 | −26.5 | 2827 | 1.65 | TRR |
| 1805 | −28.7 | 2951 | 1.63 | TSR |

TABLE 3
Summary of frequently occurring amino acid residues
in positions 33 (pos 1), 40 (pos 2) and 51 (pos 51)
in the Fc binding polypeptides disclosed herein.

| Pos1 | Pos2 | Pos3 |
|---|---|---|
| All 12 amino acids seen | All 9 amino acids seen | All 6 amino acids seen |
| 13 of 64 = Glycine (G) | 18 of 64 Arginine (R) | 20 of 64 Isoleucine (I) |
| 9 of 64 = Proline (P) | 10 of 64 Lysine (K) | 16 of 64 Arginine (R) |
| 8 of 64 = Threonine (T) | 32 of 64 | 11 of 64 Leucine (L) |
| | positively charged | |
| | 41 of 64 charged | only one Glycine (G) |
| | 57 of 64 polar | |

### Example 2

### Biacore Evaluation of Fc Binding Polypeptide VH Knock-Out Candidates and Controls

### Background

The selected 16 Fc binding polypeptide variants from the library screen were evaluated together with the control SEQ ID NO:58.

The following aspects were evaluated:
(1) Knock-out of VH3 interaction (tested through high concentration injection of Fab fragment generated from trastuzumab.
(2) Binding to trastuzumab.
(3) Kinetics/affinity assessment of Fc interaction (injection of Fc samples generated from trastuzumab)
(4) NaOH-stability (tested by reduction of trastuzumab binding after increasing number of treatments with 0.5M NaOH).

The different variants analyzed are summarized in Table 4.

TABLE 4
Overview of candidates evaluated in Biacore. Amino
acids in bold are changed compared to SEQ ID NO: 58.

| Fc binding | | Sequence at | |
|---|---|---|---|
| polypeptide | SEQ ID | aa position | |
| variant | NO: | 33/40/51 | Description |
| | 58 | SVL | Positive control for alkali stability |
| | | | and Fc interaction. Positive |
| | | | control for VH3 interaction. |
| ZGE85 | 1 | GGS | Library candidate |
| ZGE86 | 2 | GGI | Library candidate |
| ZGE87 | 3 | SRV | Library candidate |
| ZGE88 | 4 | GRL | Library candidate |
| ZGE89 | 5 | QRI | Library candidate |
| ZGE90 | 6 | ARI | Library candidate |
| ZGE91 | 7 | SGV | Library candidate |
| ZGE92 | 8 | TER | Library candidate |
| ZGE93 | 9 | GER | Library candidate |
| ZGE94 | 10 | ADL | Library candidate |
| ZGE95 | 11 | AGR | Library candidate |
| ZGE96 | 12 | EGL | Library candidate |
| ZGE97 | 13 | HKL | Library candidate |
| ZGE98 | 14 | GDV | Library candidate |
| ZGE99 | 15 | GQI | Library candidate |
| ZGE100 | 16 | QDI | Library candidate |

Generation of his-Purified Candidate Proteins

### Materials and Methods

Materials and equipment used were as follows: glycerol stocks for pAM095 (SEQ ID NO:58), and pAM200-215 (ZGE85-100, corresponding to SEQ ID NO:1-16); 2YT culture medium, Carbenicillin, IPTG, 100 ml baffled glass shake flasks, Infors HT Shaking incubator; His-GraviTrap and kit (Gytiva), PD10 GraviTraps (Gytiva).

Protein expression culture medium (2YT medium supplemented with 200 ug/ml Carbenicillin and 1 mM IPTG) was prepared and added to filled baffled shake flasks (20 ml per flask, 22 flasks in total). Each flask was inoculated with 5 μl from corresponding glycerol stock. The flasks were incubated in Infors HT shaking incubator for 24 hours at 27° C. and 190 rpm shaking, whereafter the cultures were pelleted and re-suspended in 5 ml PBS in Falcon tubes and frozen.

To generate crude Fc binding polypeptide variant lysates the frozen Falcon tubes were incubated in 80° C. water bath for 2 hours followed by pelleting of cell debris by centrifugation at 12000×g for 30 min.

Imidazole was added to the lysates to achieve the concentration recommended by the manufacturer and the samples were purified using His GraviTraps and kit according to kit instructions. Buffer exchange into HBS-P+ (0.1 M HEPES, 1.5 M NaCl and 0.5% v/v Surfactant P20) (running buffer used in Biacore) was performed using PD10 Gravitraps and kit instructions.

Concentration of buffer exchanged samples was measured using NanoDrop (Thermo Scientific) according to manufacturer's instructions. Following purification, samples were kept in refrigerator until all measurements performed and were then transferred to eppendorf tubes and kept in freezer (−20° C.).

### Ph-Scouting for Immobilization Conditions

### Materials and Methods

Materials and equipment used were as follows: Fc binding polypeptide variants ZGE85-100 (corresponding to SEQ ID NO:1-16, respectively); Biacore CM5 chips (Cytiva); Biacore Acetate-buffers (Cytiva) with different pH; Biacore 8K+ instrument (Cytiva).

The eight first variants (SEQ ID NO:1-8) were used for pH-scouting (pH 4, pH 4.5, pH 5 and pH 5.5). The pH-scout was performed on Biacore 8K+ using the standard method defined in the software. pH 5 was found to give the best surface attraction for all variants. Hence, pH 5 was selected for immobilization of variants.

### Initial Biacore Analysis of VH3 Knock-Out Variants

### Material and Methods

Materials and equipment used were as follows: CM5 sensor chips (Cytiva), Biacore NHS coupling kit (Cytiva), Fc binding polypeptide variants from above; Biacore 8K+ (Cytiva); Fab (prepared from trastuzumab in-house and comprising SEQ ID NO:198 and 199), Fc (SEQ ID NO:200) from trastuzumab.

Immobilization was performed using a standard method in Biacore software with coupling of Fc binding polypeptide variants in FC2 and activation/inactivation in FC1. Fc binding polypeptide variants were diluted in pH5 Acetate buffer at approximately 30 μg/ml. The immobilization levels did vary somewhat between different Fc binding polypeptide variants (840-1190 Ru).

### Biacore Method:

Running buffer: HBS-P+
Flow rate: 5 μl/min
Sample injection: 120 s/2 min over both Flow Cells (FC1 and FC2)
Dissociation time: 30 s
Regeneration: 10 mM Glycin-HCl pH 1.5, 30 μl/min, 2×30 s

In each run SEQ ID NO:58 was immobilized in channel1 in FC2 as internal control. Channel3-channel8 was immobilized with the candidate Fc binding polypeptides. Multiple chips were used until all candidates were tested.

Injections for each channel (cycles) were as follows: buffer, 1 g/L Fab, 40 μg/ml Fc except for immobilized ligands ZGE99-ZGE100. For immobilized ligands ZGE99-ZGE100, injections for each channel (cycles) were as follows: buffer, 1 g/L Fab.

### Results

All sensorgrams were generated as reference subtracted and the responses as the difference between baseline before injection and signal just before end of injection.

The response levels of the Fab (VH3) interaction of the Fc binding polypeptide variants are given in Table 5. The data shows that the Fc binding polypeptide variants (SEQ ID NO:1-16) exhibited a significantly reduced or abolished binding affinity for Fab, which included the VH3 from trastuzumab. A subset of the Fc binding polypeptide variants were analyzed further.

TABLE 5
Immobilization levels and response values for Fc binding polypeptide
variants following injection of 1 g/L Fab and 40 μg/ml Fc, respectively.
SEQ ID NO: 58 is a positive control for binding to VH3 from trastuzumab.

| | Immobilized | Fab 1 g/l | 40 μg/ml Fc |
|---|---|---|---|
| Immobilized | ligand | analyte binding | analyte binding |
| ligand | level (RU) | Relative (RU) | Relative (RU) |
| SEQ ID NO: 58 | 1169.5 | 2587.5 | 2650.1 |
| ZGE85 | 987.3 | 39.1 | 2336.4 |
| ZGE86 | 1031.2 | −18.4 | 2550.2 |
| ZGE87 | 1126.9 | 7.0 | 2562.2 |
| ZGE88 | 1188.7 | 8.3 | 2647.9 |
| ZGE89 | 1180.1 | 6.7 | 2615.5 |
| ZGE90 | 1159.3 | −0.9 | 2648.2 |
| ZGE91 | 889.8 | −7.0 | 1887.1 |
| ZGE92 | 1135.4 | 9.0 | 2433.6 |
| ZGE93 | 939.1 | 59.2 | 2346.0 |
| ZGE94 | 1073.2 | 23.4 | 2490.1 |
| ZGE95 | 999.5 | −0.6 | 2037.3 |
| ZGE96 | 839.4 | −3.3 | 2055.8 |
| ZGE97 | 1100.1 | 3.2 | 2546.0 |
| ZGE98 | 890.0 | 7.4 | 2217.3 |
| ZGE99 | 845.6 | 105.3 | |
| ZGE100 | 1007.7 | 12.5 | |

### Alkali Stability (0.5 MNaOH) Evaluation Using Biacore

### Materials and Methods

Materials and equipment used were as follows: CM5 sensor chips (Cytiva), Biacore NHS coupling kit (Gytiva), Fc binding polypeptide variants ZGE85-ZGE100 (corresponding to SEQ ID NO:1-16, respectively); Biacore 8K+ (Cytiva); trastuzumab, 0.5 M NaOH.

Immobilization was performed using standard method in Biacore software with coupling of Fc binding polypeptide variants in FC2 and activation/inactivation in FC1. Fc binding polypeptide variants were diluted in pH5 Acetate buffer at approximately 30 μg/ml.

For each run SEQ ID NO:58 was immobilized in channel 1 (FC1) as internal control. Channel3-channel8 immobilized with other candidates. Multiple chips used until all candidates tested.

Immobilization levels vary somewhat between different Fc binding polypeptide variants (840-1190 Ru).

### Biacore Method (Cycle):

Running buffer: HBS-P+
Flow rate: 5 μl/min
Sample injection 1 (trastuzumab 47 μg/ml): 200 s over both Flow Cells
Dissociation time 1: 60 s
Sample injection 2 (0.5 M NaOH): 300 s over both flow cells.
Regeneration: 10 mM Glycin-HCl pH 1.5, 30 μl/min, 2×30 s

This cycle was repeated more than 70 times to follow stability of trastuzumab response values.

All sensorgrams were generated as reference subtracted. Responses as difference between baseline before injection and signal just before end of injection.

### Results

Stability was visualized by plotting normalized trastuzumab response values (% of response in cycle 1) for the first cycle as set out above to the last cycle run. SEQ ID NO:58 present as control in each separate stability run. FIG. 2 shows an example plot of alkali stability for tested Fc binding polypeptide variants in comparison to SEQ ID NO:58. In each cycle, 0.5 M NaOH was injected with a contact time of 5 min at 10 μl/min.

This data showed that the candidate Fc binding polypeptides SEQ ID NO:1-16 exhibited a stability in 0.5 M NaOH similar to that of SEQ ID NO:58 (see summary in Table 8).

### Kinetics of Fc Interaction Using Immobilized Fc Binding Polypeptide Variants

### Materials and Methods

Materials and equipment used were as follows: CM5 sensor chips (Cytiva), Biacore NHS coupling kit (Cytiva), Fc binding polypeptide variants ZGE99 (SEQ ID NO:15), ZGE100 (SEQ ID NO:16), ZGE86 (SEQ ID NO:2), ZGE91 (SEQ ID NO:7), ZGE98 (SEQ ID NO:14), respectively); Biacore 8K+ (Cytiva); Fc (prepared in-house) as above.

Immobilization was performed using standard method in Biacore software with coupling of Fc binding polypeptide variants in FC2 and activation/inactivation of FC1. Fc binding polypeptide variants were diluted in pH5 Acetate buffer at approximately 30 μg/ml.

Immobilization levels varied somewhat between different Fc binding polypeptide variants (840-1190 Ru).

### Biacore Method:

Running buffer: HBS-P+
Used pre-defined method for multi-cycle kinetics on Biacore-8K+ using injections of buffer, 2.4 nM Fc, 12 nM Fc, 60 nM Fc, 300 nM Fc, 1500 nM Fc and 7500 nM Fc.

### Results

Results were analyzed using pre-defined Evaluation method Multi-cycle kinetics. Note: These data were collected using chip immobilized for evaluation of last variants in above tests.

Kinetic parameters for evaluated Fc binding polypeptide variants shown in Table 6.

The data shows that in this assay set up, the Fc-binding polypeptides ZGE99 (SEQ ID NO:15), ZGE100 (SEQ ID NO:16), ZGE86 (SEQ ID NO:2), ZGE91 (SEQ ID NO:7), ZGE98 (SEQ ID NO:14) exhibited an K_{D} value of the interaction with Fc in the range of 10⁻¹⁰ M, which is in the same range as that of the control SEQ ID NO:58.

TABLE 6
Kinetic parameters for tested Fc binding polypeptide variants immobilized
on surface. Analyte solutions: Fc. Kinetics model 1:1 binding.

| Immobilized | Kinetics | | | | Rmax | |
|---|---|---|---|---|---|---|
| ligand | Chi² (RU²) | ka (1/Ms) | kd (1/s) | KD (M) | (RU) | tc |
| SEQ ID | 2.23e+03 | 5.22e+05 | 4.53e−04 | 8.68e−10 | 971.1 | 1.25e+12 |
| NO: 58 | | | | | | |
| ZGE99 | 1.68e+03 | 5.89e+05 | 3.87e−04 | 6.57e−10 | 858.4 | 2.63e+13 |
| ZGE100 | 1.89e+03 | 5.35e+05 | 5.23e−04 | 9.78e−10 | 852.5 | 1.24e+12 |
| ZGE86 | 1.91e+03 | 5.55e+05 | 4.27e−04 | 7.68e−10 | 894.8 | 1.43e+13 |
| ZGE91 | 1.65e03 | 6.7e+05 | 5.12e−04 | 7.64e−10 | 785.7 | 8.34e+08 |
| ZGE98 | 1.75e+03 | 5.24e+05 | 4.22e−04 | 8.06e−10 | 856.4 | 2.75e+13 |

### Kinetics of Fc Interaction of the Fc Binding Polypeptides Using Immobilized Fc

### Materials and Methods

Materials and equipment used were as follows: CM5 sensor chips (Cytiva), Biacore NHS coupling kit(Cytiva), Fc binding polypeptides ZGE86 (SEQ ID NO:2), ZGE87 (SEQ ID NO:3), ZGE88 (SEQ ID NO:4), ZGE89 (SEQ ID NO:5), ZGE90 (SEQ ID NO:6) and ZGE91 (SEQ ID NO:7); Biacore 8K+ (Cytiva); Fc as above (prepared in-house).

Immobilization was performed using standard method in Biacore software with coupling of Fc in FC2 and activation/inactivation of FC1. Fc was diluted in pH5 Acetate buffer to 20 ug/ml. Immobilization levels were around 7500 Ru.

### Biacore Method:

Running buffer: HBS-P+
Used pre-defined method for multi-cycle kinetics on Biacore-8K+ using injections of buffer, 2.4 nM Fc binding polypeptide variant, 12 nM Fc binding polypeptide variant, 60 nM Fc binding polypeptide variant, 300 nM Fc binding polypeptide variant, 1500 nM Fc binding polypeptide variant. 8 Fc binding polypeptide variants evaluated in parallel. Results were analyzed using pre-defined Evaluation method Multi-cycle kinetics.

### Results

Kinetic parameters for evaluated Fc binding polypeptide variants shown in Table 7.

The data shows that in this assay set up, the Fc-binding polypeptides, ZGE86 (SEQ ID NO:2), ZGE87 (SEQ ID NO:3), ZGE88 (SEQ ID NO:4), ZGE89 (SEQ ID NO:5), ZGE90 (SEQ ID NO:6) and ZGE91 (SEQ ID NO:7) exhibited an K_{D} value of the interaction with Fc in the range of 10⁻⁷ M, which is in the same range as that of the control SEQ ID NO:58.

TABLE 7
Kinetic parameters for tested Fc binding polypeptide variants with Fc immobilized
on surface. Immobilized ligand: Fc 20 μg/ml pH 5. Kinetics model 1:1 binding.

| Analyte 2 | Kinetics | | | | Rmax | |
|---|---|---|---|---|---|---|
| Solution | Chi² (RU²) | ka (1/Ms) | kd (1/s) | KD (M) | (RU) | tc |
| SEQ ID | 1.25e+01 | 2.10e+05 | 3.50e−02 | 1.67e−07 | 248.0 | 2.67e+09 |
| NO: 58 | | | | | | |
| ZGE86 | 1.43e+01 | 1.90e+05 | 2.90e−02 | 1.52e−07 | 257.9 | 2.66e+09 |
| ZGE87 | 1.55e+01 | 1.89e+05 | 3.41e−02 | 1.81e−07 | 272.4 | 2.85e+09 |
| ZGE88 | 1.78e+01 | 2.35e+05 | 3.05e−02 | 1.30e−07 | 278.4 | 1.23e+08 |
| ZGE89 | 1.54e+01 | 2.04e+05 | 3.64e−02 | 1.78e−07 | 271.0 | 1.27e+08 |
| ZGE90 | 1.48e+01 | 2.22e+05 | 3.79e−02 | 1.71e−07 | 262.4 | 1.33e+08 |
| ZGE91 | 1.46e+01 | 1.86e+05 | 3.35e−02 | 1.80e−07 | 263.1 | 3.22e+12 |

### Summary of Results and Comments

Table 8 summarizes the results from the detailed Biacore evaluation as described above. It was observed that all evaluated Fc binding polypeptides ZGE85-100, corresponding to SEQ ID NO:1-16, exhibited significantly reduced or abolished ability to bind to VH3 of trastuzumab, Furthermore, the analyzed Fc binding polypeptides showed a binding affinity for Fc of trastuzumab as well as stability as evaluated in 0.5 M NaOH in the same range as the control SEQ ID NO:58.

Based on the obtained results, three variants (ZGE90 (SEQ ID NO:6); ZGE91 (SEQ ID NO:7); and ZGE96 (SEQ ID NO:12) were selected for hexamer construction and further analysis (see Example 3). In selection, priority was given to complete absence of VH3 interaction and high alkali stability.

TABLE 8
Summary of results from detailed Biacore evaluation of Fc binding polypeptide variants.
Variants in bold were selected for hexamer construction and further analysis.

| | | | | | FC | | | |
|---|---|---|---|---|---|---|---|---|
| | Fab | | | | affinity | | | |
| | binding | | FC ka* | FC kd* | KD* | | Stability | |
| | RU (% of | | (% of | (% of | (% of | | 0.5M NaOH | |
| | SEQ ID | | SEQ ID | SEQ ID | SEQ ID | Bivalent | (% of SEQ | |
| Variant | NO: 58) | | NO: 58) | NO: 58) | NO: 58) | aff. | ID NO: 58) | |
| ZGE85 | 39.1 | (1.6) | — | — | — | — | 62.6 | |
| ZGE86 | −18.4 | (0) | 90.5 | 120.7 | 109.9 | 113% | 89 | (100x) |
| | | | | | | (0.77 nM) | 98.6 | (30x) |
| ZGE87 | 7.0 | (0.27) | 90 | 102.6 | 92.3 | — | 100.3 | (64x) |
| ZGE88 | 8.3 | (0.32) | 111.9 | 114.8 | 128.5 | — | 100.1 | (64x) |
| ZGE89 | 6.7 | (0.26) | 97.1 | 96.2 | 93.8 | — | 100.2 | (64x) |
| ZGE90 | −0.9 | (0) | 105.7 | 92.3 | 97.7 | — | 99.9 | (64x) |
| ZGE91 | −7.0 | (0) | 88.6 | 104.5 | 92.8 | 114% | 98.4 | (64x) |
| | | | | | | (0.76 nM) | 100 | (30x) |
| ZGE92 | 9.0 | (0.35) | 83.1 | 75.3 | 62.3 | — | 95.1 | (64x) |
| ZGE93 | 59.2 | (2.5) | — | — | — | — | 98.6 | (30x) |
| ZGE94 | 23.4 | (1) | — | — | — | — | 99.8 | (30x) |
| ZGE95 | −0.6 | (0) | 98.5 | 102 | 100 | — | 99.5 | (30x) |
| ZGE96 | −3.3 | (0) | 76.4 | 85.4 | 65 | — | 99 | (30x) |
| ZGE97 | 3.2 | (0.14) | — | — | — | — | 100.7 | (30x) |
| ZGE98 | 7.4 | (0.32) | 82 | 130.9 | 106.4 | 107% | 98.8 | (30x) |
| | | | | | | (0.81 nM) | | |
| ZGE99 | 105.3 | (4.6) | — | — | — | 132% | 100 | (30x) |
| | | | | | | (0.66 nM) | | |
| ZGE100 | 12.5 | (0.6) | — | — | — | 88.8% | 99.5 | (30x) |

(0.98 nM)

*Monovalent affinity; FC immobilized

### Example 3

### Evaluation of Fc Binding Polypeptides VH Knock-Out Hexamer Candidates and Controls

### Expression and Coupling of Four Variants in Hexameric Form

### Plasmids and Cell-Banks

A subset of hexamer candidate variants according to general formula SEQ ID NO:207 were prepared. In particular, four sequences (ZGE90 (SEQ ID NO:6), ZGE91 (SEQ ID NO:91), ZGE96 (SEQ ID NO:12) and SEQ ID NO:58) as hexamers, which comprise the sequences according to SEQ ID NO:203-206, respectively, were ordered as DNA synthesis plasmids from ATUM (Newark, CA, USA) in the pJ401 plasmid backbone. The plasmids were transformed into chemically competent E. coli K12-017 cells and research cell-banks were made according to standard procedures.

### Fermentation

1 L fermentations was performed with the four abovementioned hexamers candidates. The results from protein expression are shown in Table 9.

TABLE 9
Samples were prepared by heat treatment in heat-block at 85° C., 5 min.

| | | | Expression |
|---|---|---|---|
| Name | SEQ ID NO: | Scale | [g/L] |
| ZGE90-6 | 203 | 1 L | 8.9 |
| ZGE91-6 | 204 | 1 L | 14.7 |
| ZGE96-6 | 205 | 1 L | 11.1 |
| Hexamer of | 206 | 1 L | 12.0 |
| SEQ ID | | | |
| NO: 58 | | | |

The products of the fermentations were purified on IgG Sepharose® as capture followed by Source™ 15Q as polishing step.

Coupling was made with epoxy coupling and the ligand density of the coupled prototypes was measured with amino acid analysis (AAA) by QC, and dynamic binding capacity (DBC) was measured with 1 mL cubed gel, see Table 10.

TABLE 10
Ligand density, Free His, Lys and DBC measurements
at 6 min residence time and pH at elution.

| | | Dry | | | | DBC | |
|---|---|---|---|---|---|---|---|
| | | Weight | Ligand | | | Qb10% | |
| | SEQ | Increase, | density, | Free | Free | 6 min rt, | pH at |
| | ID | mg/mL | mg/mL | His, | Lys, | mg/mL | peak |
| Name | NO: | gel | gel | % | % | gel | elution |
| ZGE90-6 | 203 | 21.9 | 18.6 | 101 | 101 | 63.6 | 4.0 |
| ZGE91-6 | 204 | 17.6 | 16 | 98 | 101 | 66.0 | 4.0 |
| ZGE96-6 | 205 | 14.5 | 13.8 | 95 | 102 | 60.3 | 4.0 |
| Hexamer | 206 | NA | 16.1 | 95 | 100 | 63.8 | 3.8 |
| of SEQ | | | | | | | |
| ID NO: 58 | | | | | | | |

### Evaluation of Hexameric Variants by Chromatography

Coupled variants were initially tested for DBC with trastuzumab and negative binding for a Fab fragment cleaved from trastuzumab. None of the candidate hexamers showed binding to the Fab. Moreover, a pH gradient was run to analyze pH at elution and alkaline stability with an accelerated alkaline stability study. The results are shown in Table 11.

Alkaline Stability with Accelerated Alkaline Stability Study

Accelerated alkaline stability studies was performed on coupled hexamers with trastuzumab as testing substance. The DBC was initially measured for the candidate hexamers and a reference resin. Then, resins were soaked in 0.5 M NaOH for 4 h contact time (which is comparable to 16 cycles of 15 min contact time). After the NaOH soak, the resins were neutralized in running buffer and a new DBC measurement was performed. This cycling was then iterated for up to 24 h total contact time. The results are shown in Table 11.

TABLE 11
Accelerated alkaline studies. Incubation in 0.5M NaOH,
4 h followed by DBC measurements at 6 min residence time.
The study is cycled up to 24 h total incubation time,
corresponding to 96 cycles of 15 min contact time.

| | | | | | | | | Residual |
|---|---|---|---|---|---|---|---|---|
| | | | | | | | | capacity |
| Candidate | 0 h | 4 h | 8 h | 12 h | 16 h | 20 h | 24 h | [%] |
| ZGE90-6 | 60.6 | 59.9 | 60.4 | 59.7 | 59.7 | 59.3 | 53.5 | 88% |
| ZGE91-6 | 61.7 | 58.5 | 62.7 | 61.9 | 61.9 | 61.4 | 56.2 | 91% |
| ZGE96-6 | 62.6 | 60.4 | 60.5 | 58.7 | 58.7 | 57.3 | 51.4 | 82% |
| Hexamer | 65.9 | 58.4 | 60.8 | 60.5 | 60.5 | 60.4 | 59.1 | 90% |
| of SEQ ID | | | | | | | | |
| NO: 58 | | | | | | | | |

### Results

All tested candidate hexamers exhibited a residual capacity of over 80% after 24 hours.

### Binding Characteristics in Biacore

### Materials and Methods

Hexameric and monomeric Fc binding polypeptide variants, Biacore CM5 chips; EDC/NHS coupling kit, Biacore Acetate-buffers with different pH; PBS-P+ running buffer (Biacore); 10 mM Glycin-HCl pH 1.5; Fab fragment of trastuzumab, Fc, trastuzumab and unknown antibody (which does not comprise VH3) stock solutions; Biacore 8K+ instrument.

Fc binding polypeptide variants were immobilized on Biacore CM5 chip FC2 using standard EDC/NHS coupling method (FC1 activated and blocked) with Fc binding polypeptide variants diluted in Biacore acetate buffer with pH5 (410 s contact time). Coupling per channel according to Table 12 below.

TABLE 12
Summary of binding characteristics of monomer and hexamers of
selected Fc binding polypeptides.

| | | | Concentration | |
|---|---|---|---|---|
| | Fc-binding | | in Acetate- | Coupled |
| | polypeptide | SEQ ID | buffer pH5 | amount |
| Channel | variant | NO: | (ug/ml) | (Ru) |
| 1 | Hexamer of SEQ | 207 | 60 | 5461 |
| | ID NO: 58 | | | |
| 2 | ZGE90-6 | 203 | 48 | 6469 |
| 3 | ZGE91-6 | 204 | 61 | 5870 |
| 4 | ZGE96-6 | 206 | 51 | 5682 |
| 5 | SEQ ID NO:58 | 58 | 22 | 1022 |
| 6 | ZGE86 | 2 | 17 | 900 |
| 7 | ZGE90 | 6 | 17 | 1154 |

Next a start-up cycle using 125 ng/ml trastuzumab was run (120 s contact time at 10 μl/min) followed by regeneration using a 30 s pulse of 10 mM Glycin-HCl at 30 μl/min. The different analytes were then injected over the surface using the same method. Injections summarized in Table 13. The results are displayed as reference subtracted sensorgrams (FC2-FC1).

TABLE 13
Summary of injections

| Injection | Sample |
|---|---|
| 1 | 1000 μg/ml Fab (Fab fragment of trastuzumab; |
| | comprises VH3) |
| 2 | 40 μg/ml Fc (Prepared by digestion from |
| | trastuzumab) |
| 3 | 50 μg/ml trastuzumab (comprises a VH3 |
| | region) |
| 4 | 500 μg/ml of unknown antibody, which does not |
| | comprise VH3 |

### Results

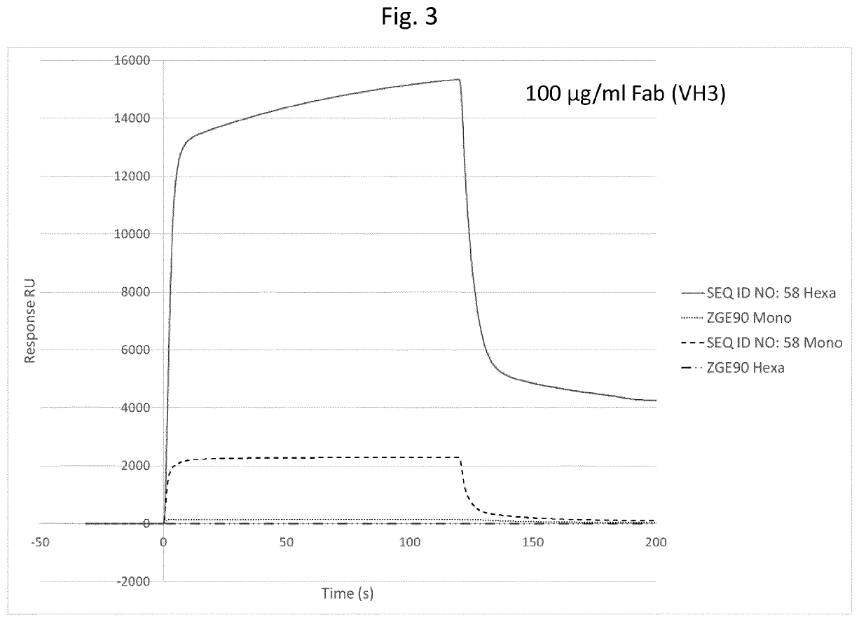

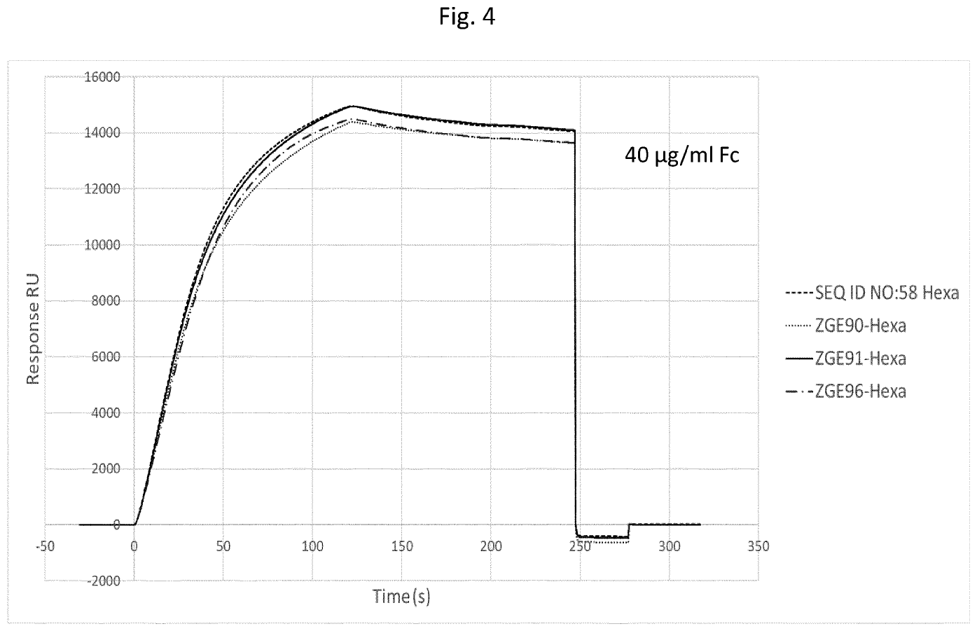

FIG. 3 shows Biacore binding curves (reference-subtracted) following injection of 1 g/L Fab from trastuzumab.

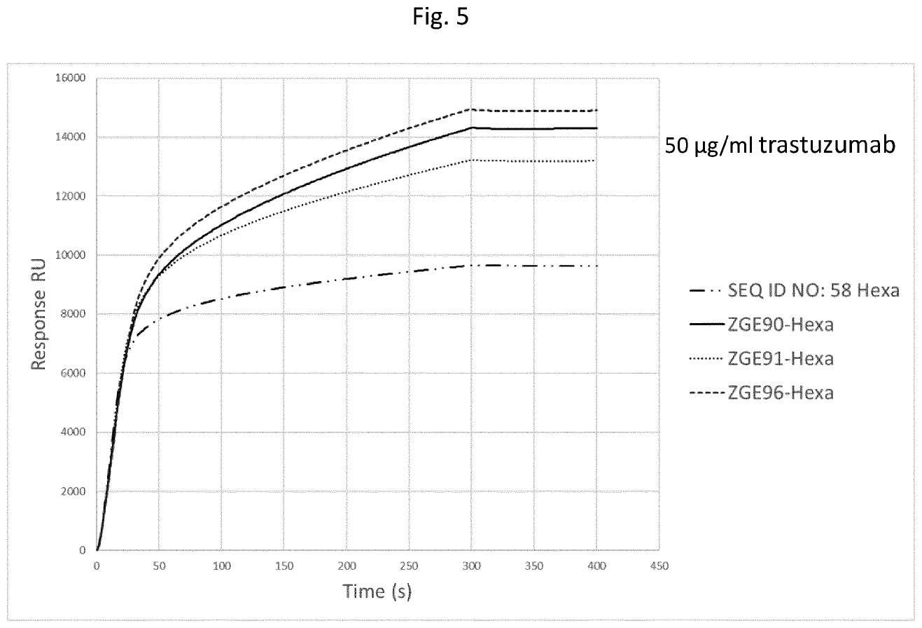

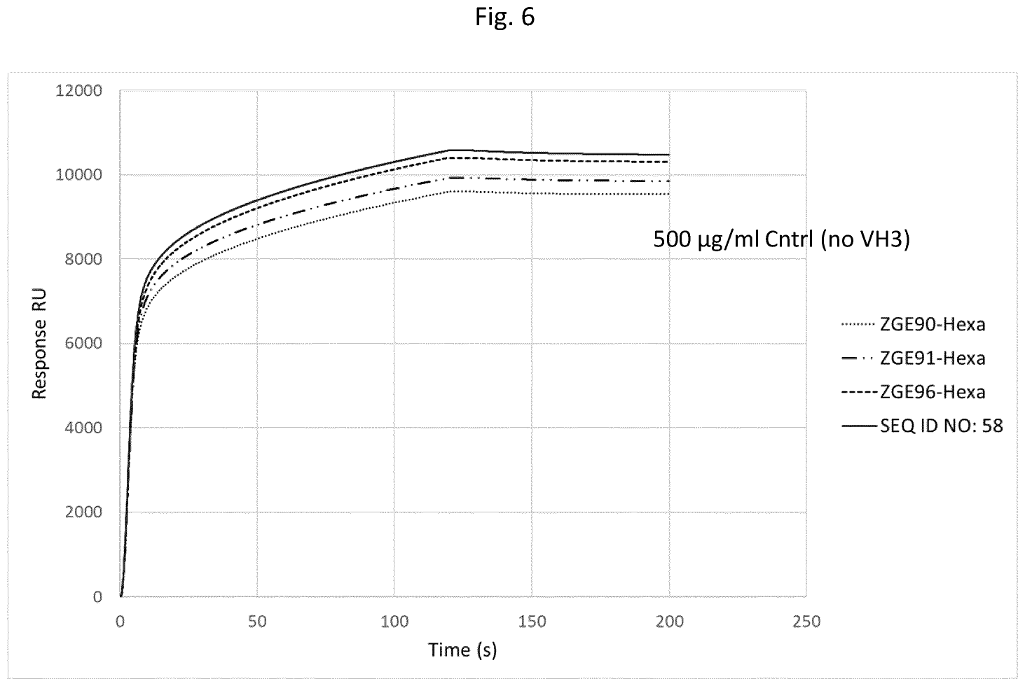

Binding to the three different Fc containing targets (Fc only, trastuzumab and a monoclonal antibody which does not contain VH3) was tested. The Biacore curves can be found in FIG. 4 to 6 (FIG. 4: 40 μg/ml Fc; FIG. 5: 50 μg/ml trastuzumab; FIG. 6: 500 μg/ml of said monoclonal antibody which does not contain VH3). As can be seen, binding response is roughly inversely proportional to the amount of ligand immobilized on chip, with one major exception, SEQ ID NO:58 shows significantly reduced binding to trastuzumab. These results prompted a more thorough investigation of the relation between coupling level and binding response (capacity) for different targets using SEQ ID NO:58 (with dual interaction) and ZGE91 (with pure Fc interaction).

SEQ ID NO:58 and ZGE91 were immobilized (coupled) at different levels and Fc (400 mg/L), Fab (76.8 mg/L), trastuzumab (470 mg/L) and said monoclonal antibody which does not contain VH3 (510 mg/L) were injected at high concentrations over the surfaces.

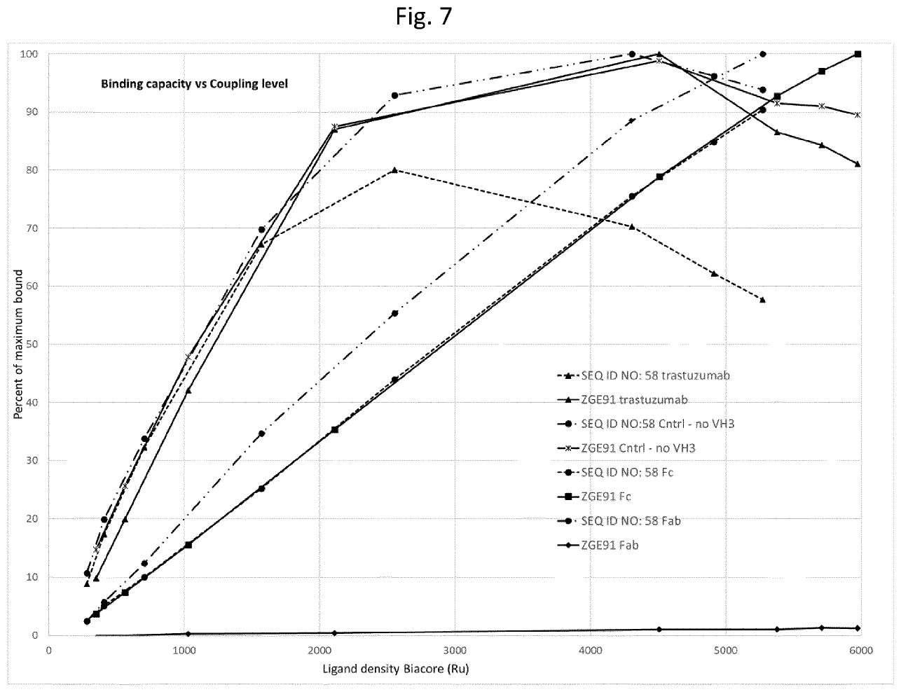

A plot showing binding level as a function of coupled ligand level for the two hexameric ligands and the different targets can be found in FIG. 7.

Interestingly, the VH3 negative variant ZGE91 (SEQ ID NO:7) showed a higher binding capacity than SEQ ID NO:58, which binds the VH3 and Fc regions of trastuzumab.

### NaOH Stability in Biacore Assay

### Materials and Methods

Hexameric and monomeric Fc binding polypeptide variants, Biacore CM5 chips; EDC/NHS coupling kit, Biacore Acetate-buffers with different pH; PBS-P+ running buffer (Biacore); 10 mM Glycin-HCl pH 1.5; trastuzumab and stock solution; Biacore 8K+ instrument.

Fc binding polypeptide variants were immobilized on Biacore CM5 chip FC2 using standard EDC/NHS coupling method (FC1 activated and blocked) with Fc binding polypeptide variants diluted in Biacore acetate buffer with pH5/pH4.5 (205 s contact time). Coupling per channel was according to Table 14 below. The experiment aimed to reach close to 1000 Ru immobilized for each variant.

TABLE 14
Coupling per channel.

| | | | | Contact | |
|---|---|---|---|---|---|
| | Fc binding | SEQ | Concentration | time in | Coupled |
| | polypeptide | ID | in Acetate- | coupling | amount |
| Channel | variant | NO: | buffer (μg/ml) | (s) | (Ru) |
| 1 | Hexamer of | 207 | 11.25/pH4.5 | 205 | 1004 |
| | SEQ ID | | | | |
| | NO:58 | | | | |
| 2 | ZGE90-6 | 203 | 7.5/pH5 | 205 | 1072 |
| 3 | ZGE91-6 | 204 | 7.5/pH5 | 205 | 750 |
| 4 | ZGE96-6 | 205 | 7.5/pH5 | 205 | 872 |
| 5 | ZGE91 | 7 | 15/pH5 | 205 | 832 |
| 6 | ZGE95 | 11 | 15/pH5 | 205 | 924 |
| 7 | ZGE96 | 12 | 15/pH5 | 205 | 902 |

### Biacore Method

Injection of 96 μg/ml trastuzumab at 10 μl/min and 300 s contact time, 2×10 mM Glycin-HCl pH 1.5 at 30 μl/min and 30 s contact time, 0.5M NaOH at 10 μl/min and 600 s contact time. The cycle repeated 100×. Results showed are based on reference subtracted data (from Cycle 2 onward).

### Results

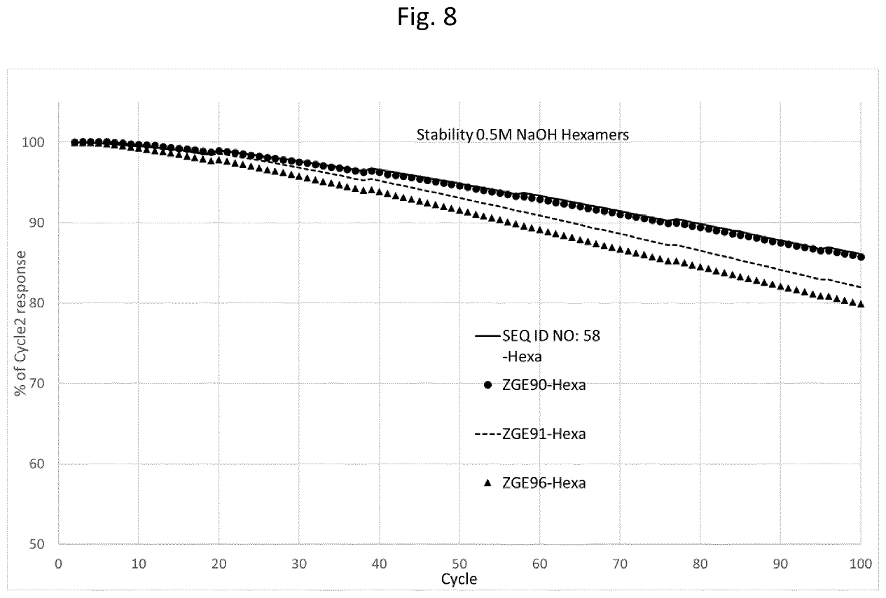

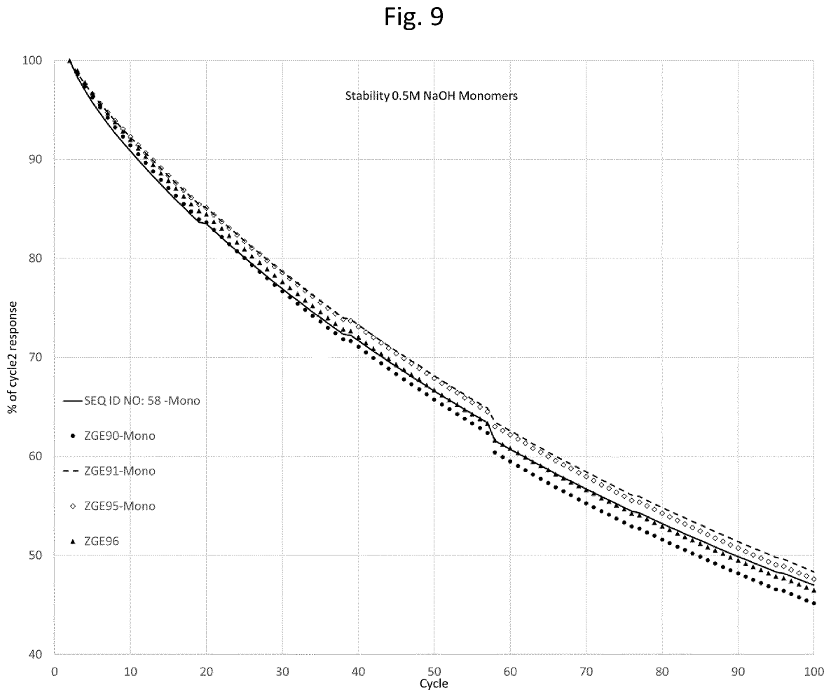

Results are shown be found in FIGS. 7 and 8. Coupling level of ligands was adjusted to be in the linear response range. Note that differences in the exact coupling level can have some impact on obtained stability plots.

Based on the Biacore data (FIG. 7), it seems plausible that ZGE91-6 (SEQ ID NO:204) exhibits a high optimal ligand density and hence a potential for increased capacity. The data in FIG. 7 also indicates that optimal coupling level is dependent on the size of the target protein. It may be that that complex bi-specific formats or large Fc fusion proteins would benefit from a product with lower ligand density.

### Example 4

### Evaluation of Fc Binding Polypeptides VH Knock-Out Candidates in Different Scaffolds

It is possible to introduce the mutations in positions 33, 40 and 51 in a different scaffold background. For instance SEQ ID NO:59 may be modified with the amino acid residues according to Table 2 in the three positions. The resulting Fc binding polypeptides are listed in FIG. 10 as SEQ ID NO:208-264.

Additionally, a selection of mutations as disclosed above may be introduced into any scaffolds, such as any one of domains A (SEQ ID NO:62), B (SEQ ID NO:61), C (SEQ ID NO:60), D (SEQ ID NO:63) and E (SEQ ID NO:64) of SpA or derivatives thereof, such as domain Z (SEQ ID NO:59) or variants thereof, such as SEQ ID NO:58.

Evaluation and analysis was performed essentially as described in Example 2, section entitled “Initial Biacore analysis of VH3 knock-out and retained Fc binding”.

It is expected that the Fc binding polypeptides SEQ ID NO:208-264 and 269-304 exhibit Fc binding affinity in the same range as SEQ ID NO:59 and significantly reduced or abolished affinity for VH3 of trastuzumab compared to SEQ ID NO:59. Such a comparison can also be made with SEQ ID NO:58.

For the purpose of showing that the mutations in positions 33, 40 and 51 have the same effect in any scaffold background, three sets of mutations for positions 33, 40 and 51 according to the above, A/R/I, S/G/V, and A/G/R, were introduced into domains A (ZGE0206) (SEQ ID NO:62), B (ZGE0207) (SEQ ID NO:61), C (ZGE0208) (SEQ ID NO:60), D (ZGE0205) (SEQ ID NO:63) and E (ZGE0204) (SEQ ID NO:64), Zwt (ZGE0209) (SEQ ID NO 59) as well as modified versions of protein A such as ZGE0201 (SEQ ID NO 265), ZGE0202 (SEQ ID NO 266), and ZGE0203 (SEQ ID NO 267), and modified version of Z such as Z var (ZGE0210) (SEQ ID NO 268). These constructs were tested in a Biacore as disclosed above, with small modifications as indicated below.

Monomeric protein A or protein A derived IgG binding polypeptide variants according to the above (in Biacore 10 mM Acetate-buffers (pH4, pH4.5, pH5 or pH5.5) were immobilized on Biacore CM5 chips FC2 and FC1 as reference (activated and deactivated) using standard EDC/NHS coupling method. Experiments used PBS-P+ running buffer (Biacore); regeneration buffer 10 mM Glycin-HCl pH 1.5; Target molecules trastuzumab, adalimumab and adalimumab Fc fragment at concentrations 0, 5, 25, 50, 100, 200, 400, 800, 1600 nM, and trastuzumab Fab fragment at concentrations 0, 50, 100, 200, 400, 800, 1600 nM were used with a Biacore 8K+ instrument.

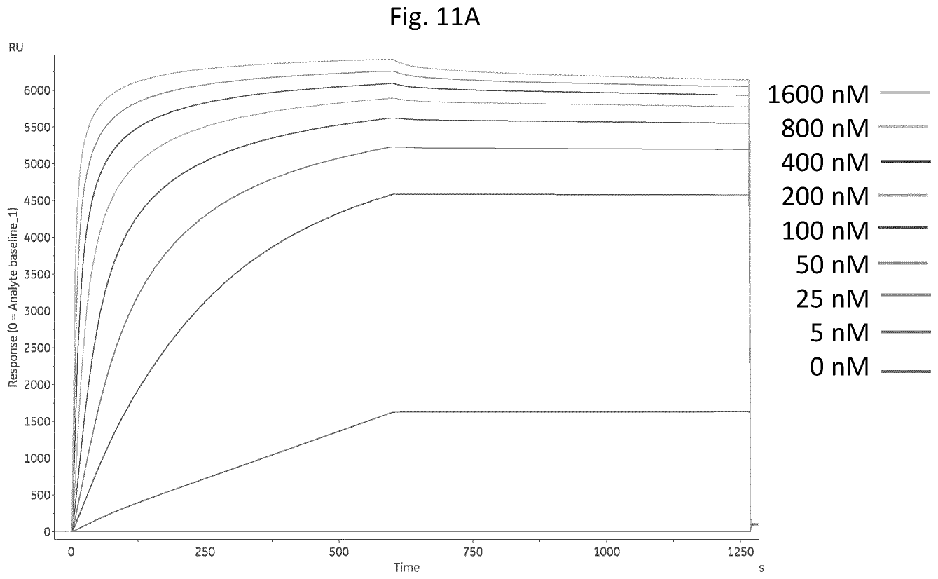

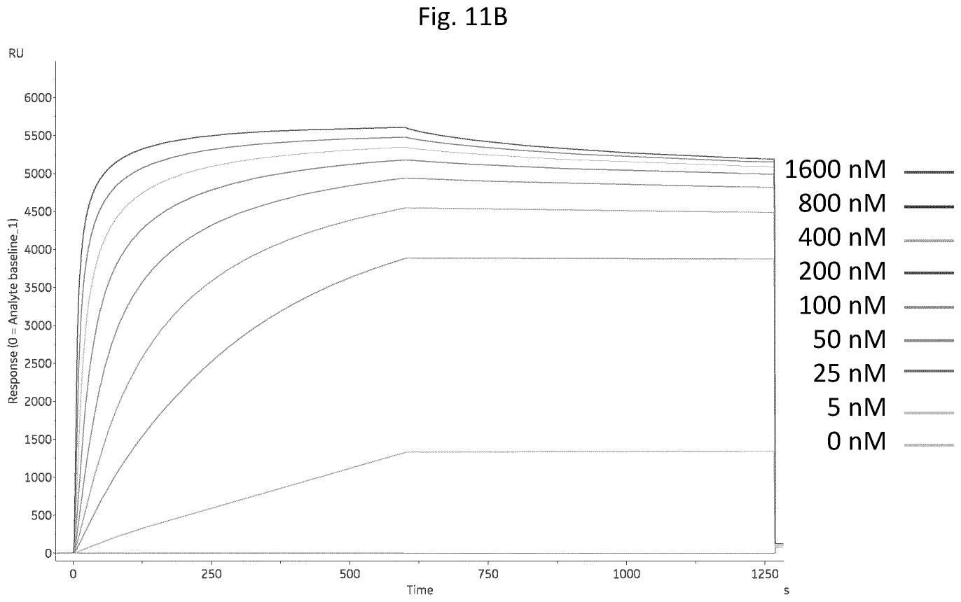

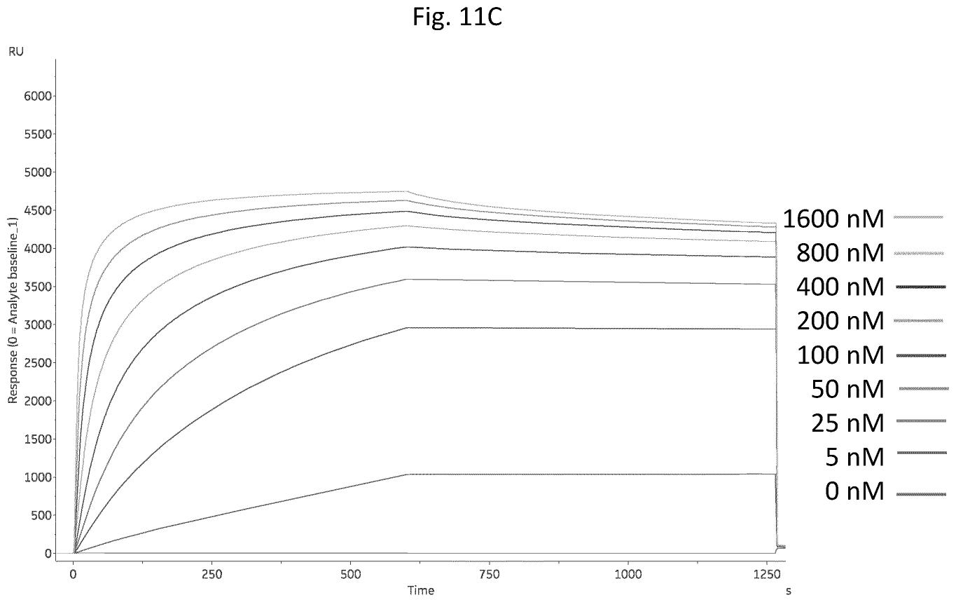

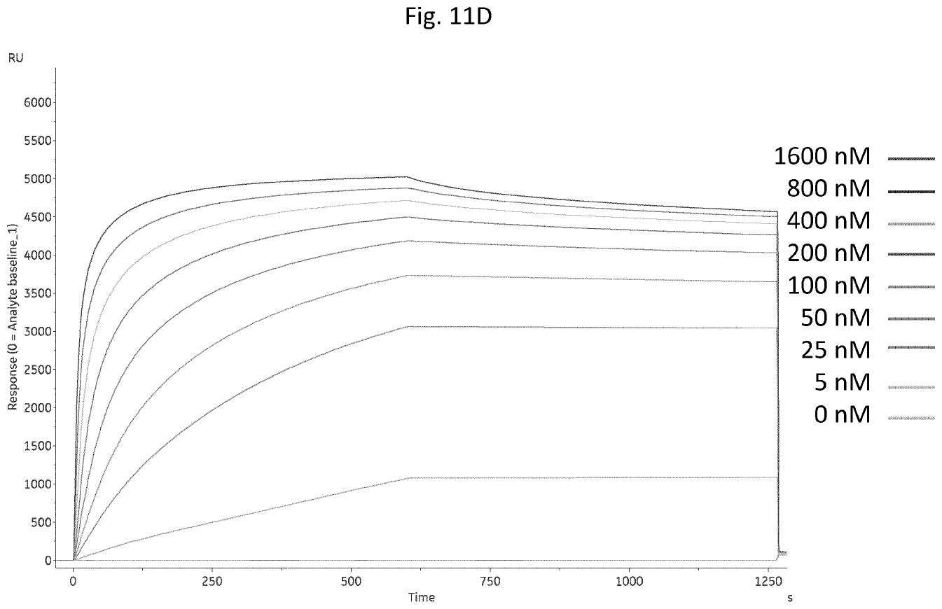

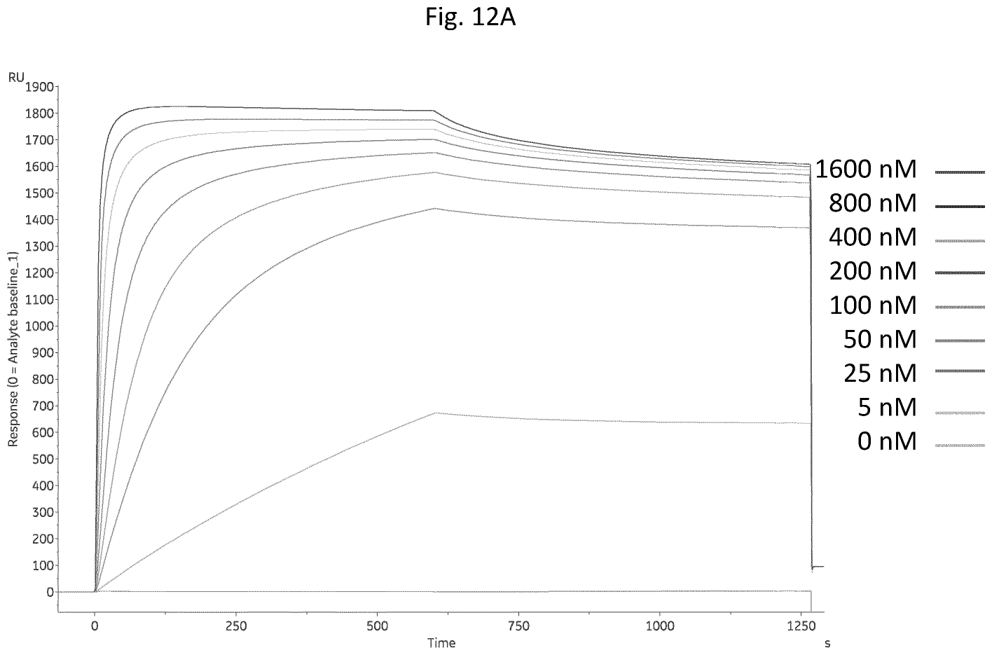

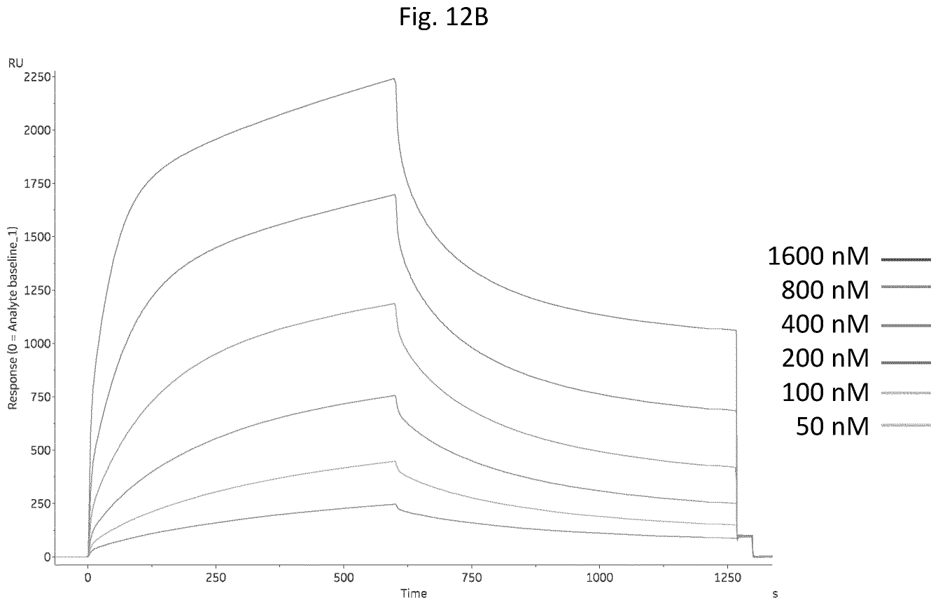

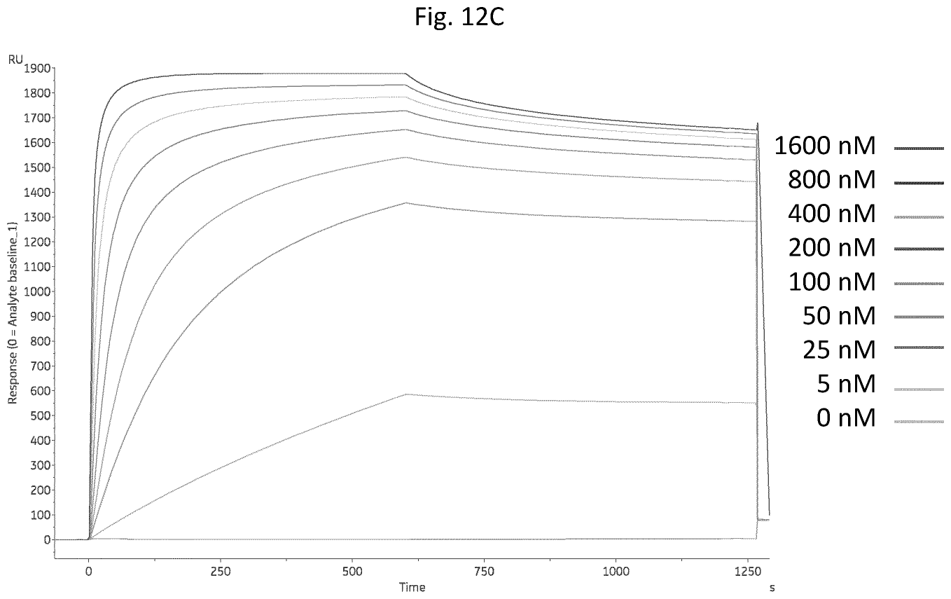

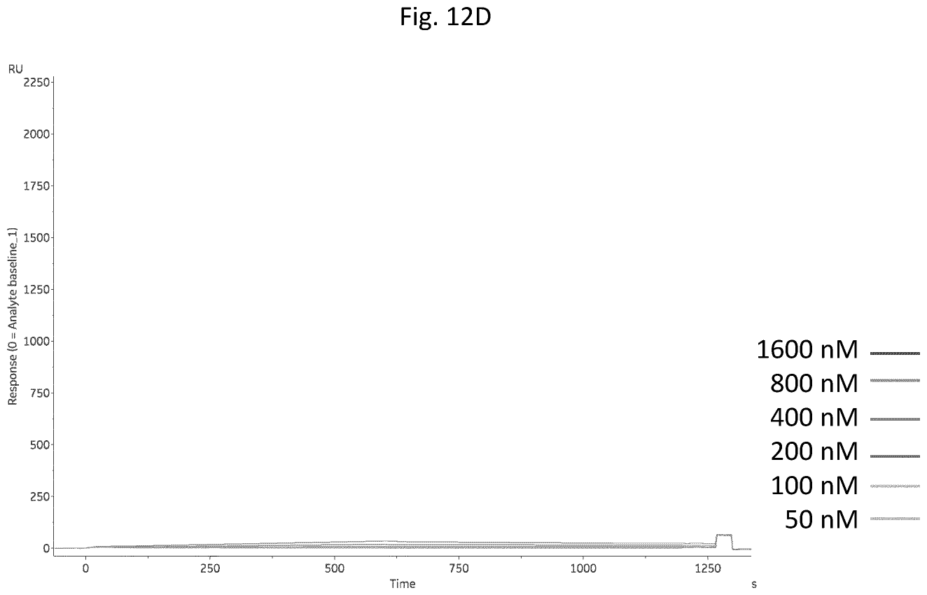

Each cycle used a flowrate of 10 μl/min with 600 seconds association and disassociation of each target molecule and concentration followed by regeneration using two 30 s pulses of 10 mM Glycin-HCl at 30 μl/min. The results are presented in Table 15 below for max response of 800 nM of each target molecule, wherein the results have been rounded to the nearest 50 ru. Exemplary samples are displayed as reference subtracted sensorgrams (FC2-FC1) in FIGS. 11 and 12.

TABLE 15
Binding in Ru to trastuzumab, adulimumab, Fc fragment from adalimumab,
Fab fragment from trastuzumab, for different scaffolds.

| | SEQ | | | Fc | Fab | Immobilized |
|---|---|---|---|---|---|---|
| Sample | ID | Trastuzumab | Adalimumab | fragment | fragment | ligand level |
| No. | NO | (Ru) | (Ru) | (Ru) | (Ru) | (Ru) |
| ZGE0201 | 265 | 5200 | 5050 | 1700 | 150 | 860 |
| ZGE0211 | 269 | 5550 | 5900 | 2400 | 50 | 1110 |
| ZGE0221 | 279 | 3550 | 3800 | 1400 | 50 | 710 |
| ZGE0231 | 289 | 3800 | 4050 | 1500 | 50 | 780 |
| ZGE0202 | 266 | 5850 | 5250 | 1800 | 50 | 1000 |
| ZGE0212 | 270 | 5700 | 6000 | 2400 | 0 | 1170 |
| ZGE0222 | 280 | 5250 | 5500 | 2000 | 0 | 1050 |
| ZGE0232 | 290 | 3600 | 3800 | 1400 | 0 | 840 |
| ZGE0203 | 267 | 4700 | 3250 | 1250 | 100 | 1120 |
| ZGE0213 | 271 | 3250 | 3550 | 1650 | 0 | 1200 |
| ZGE0223 | 281 | 1355 | 1400 | 650 | 0 | 1280 |
| ZGE0233 | 291 | 1400 | 1550 | 700 | 0 | 1230 |
| Domain E | | | | | | |
| ZGE0204 | 64 | 5050 | 4200 | 1450 | 850 | 780 |
| ZGE0214 | 272 | 3850 | 4100 | 1650 | 0 | 780 |
| ZGE0224 | 282 | 3550 | 3650 | 1350 | 0 | 740 |
| ZGE0234 | 292 | 2750 | 2950 | 1100 | 0 | 800 |
| Domain D | | | | | | |
| ZGE0205 | 63 | 5350 | 4600 | 1600 | 750 | 830 |
| ZGE0215 | 273 | 4700 | 5000 | 1950 | 0 | 930 |
| ZGE0225 | 283 | 4100 | 4200 | 1500 | 0 | 810 |
| ZGE0235 | 293 | 4800 | 5050 | 1850 | 0 | 1000 |
| Domain A | | | | | | |
| ZGE0206 | 62 | 4850 | 4050 | 1350 | 1150 | 710 |
| ZGE0216 | 274 | 4350 | 4550 | 1850 | 0 | 1140 |
| ZGE0226 | 284 | 1500 | 1500 | 600 | 0 | 810 |
| ZGE0236 | 294 | 2550 | 2700 | 1050 | 0 | 750 |
| Domain B | | | | | | |
| ZGE0207 | 61 | 6250 | 5500 | 1750 | 1700 | 930 |
| ZGE0217 | 275 | 4650 | 4900 | 1850 | 0 | 1170 |
| ZGE0227 | 285 | 3300 | 3300 | 1300 | 0 | 940 |
| ZGE0237 | 295 | 3050 | 3250 | 1250 | 0 | 800 |
| Domain C | | | | | | |
| ZGE0208 | 60 | 6000 | 50000 | 1750 | 3000 | 950 |
| ZGE0218 | 276 | 5450 | 5650 | 2100 | 50 | 1000 |
| ZGE0228 | 286 | 3300 | 3200 | 1300 | 0 | 1060 |
| ZGE0238 | 296 | 3650 | 3750 | 1500 | 0 | 990 |
| Z wt | | | | | | |
| ZGE0209 | 59 | 4400 | 4000 | 1600 | 50 | 970 |
| ZGE0219 | 277 | 5100 | 5400 | 2000 | 0 | 1180 |
| ZGE0229 | 287 | 3350 | 3550 | 1400 | 0 | 930 |
| ZGE0239 | 297 | 2950 | 3150 | 1200 | 0 | 850 |
| Z var | | | | | | |
| ZGE0210 | 268 | 4600 | 4300 | 1750 | 50 | 1090 |
| ZGE0220 | 278 | 4950 | 5250 | 2000 | 0 | 1230 |
| ZGE0230 | 288 | 2650 | 2850 | 1150 | 0 | 930 |
| ZGE0240 | 297 | 3150 | 3350 | 1300 | 0 | 710 |

Each scaffold was tested in a set of four and is grouped accordingly in the table above: 1) wt, 2) with mutation A/R/I, 3) with mutation S/G/V, and 4) with mutation A/G/R. Thus, as an example, for domain C the test set is according to the following: 1) ZGE0208 (wt), 2) ZGE0218 (A/R/I), 3) ZGE228 (S/G/V), 4) ZGE238 (A/G/R).

In general, the response for Fc-binding is lower than the response for the full antibody, as the Fc fragment is smaller than the full antibody. The response in a Biacore system is directly related to the change in mass concentration on the surface, so that molar responses (i.e. responses for a given number of molecules) are proportional to the size of the molecule involved. Thus, a given number of molecules binding to the surface will give a lower response if the molecule is small.

As is evident from Table 15, and FIGS. 11 and 12, the tested mutations had the effect of substantially maintaining the Fc binding activity, but substantially abolishing the Fab binding activity for all domains and versions thereof tested, in comparison with the wildtype or a mutated version of the SpA domain lacking the above specified mutations for positions 33, 40 and 51.

Thus, it is shown that the mutations as disclosed herein for positions 33, 40 and 51, as defined above, retains the inherent ability to bind to the Fc region of antibodies/immunoglobulins, in particular to the Fc region of the antibody subclasses IgG1, IgG2 and IgG4, and show a reduced or abolished binding affinity for the VH3 region of the antibody trastuzumab, for all Protein A scaffolds, such as naturally occurring SpA domains, as well as versions of the same.

### ITEMIZED LIST OF EMBODIMENTS

1. Fc binding polypeptide derived from a Staphylococcus Protein A (SpA) or any domain thereof, wherein said polypeptide has binding affinity to an Fc region of an immunoglobulin, and has lower binding affinity for a VH3 region of trastuzumab compared to the binding affinity of SEQ ID NO:59 for the same VH3 region.
2. Fc binding polypeptide comprising a sequence A, which Sequence A consists of an amino acid sequence selected from i), ii) and iii), wherein i), ii) and iii) are defined as follows:

i)
(SEQ ID NO: 65)
EX₂₅QX₂₇X₂₈X₂₉X₃₀IX₃₂X₃₃LX₃₅X₃₆X₃₇PSX₄₀SX₄₂X₄₃X₄₄LX₄₆EAX₄₉
X₅₀X₅₁NX₅₃X₅₄

wherein, independently from each other,
X₂₅ is selected from E and D;
X₂₇ is selected from R and H;
X₂₈ is selected from N, A, S, H and W;
X₂₉ is selected from A, G and K;
X₃₀ is selected from F and A;
X₃₂ is selected from Q and H;
X₃₅ is selected from K, R and H;
X₃₆ is selected from D and H;
X₃₇ is selected from D and E;
X₄₂ is selected from A, K, L, R and T;
X₄₃ is selected from N, E, A, K and S;
X₄₄ is selected from L, I and V;
X₄₆ is selected from A, G and K;
X₄₉ is selected from K, Q and R;
X₅₀ is selected from K and R;
X₅₃ is selected from D, E and K; and
X₅₄ is selected from A and S;
ii) an amino acid sequence which has at least 83% identity to a sequence defined by i)
iii) an amino acid sequence which has at least 70% identity to any sequence selected from the group consisting of: residues 24-54 in SEQ ID NO:58, residues 24-54 in SEQ ID NO:59, residues 24-54 in SEQ ID NO:60, residues 24-54 in SEQ ID NO:61, residues 24-54 in SEQ ID NO:62, residues 27-57 in SEQ ID NO:63 and residues 22-52 in SEQ ID NO:64,
   wherein additionally, in each of i), ii) and iii) independently from each other,
X₃₃ is selected from T, S, G, Q, A, E, H, R, P, D, K and N;
X₄₀ is selected from E, G, R, D, K, Q, N, H and S; and
X₅₁ is selected from L, V, S, I, R and G;
   with the proviso that when X₅₁ is L then X₃₃X₄₀ is selected from AD, HK, EG, ER, GR, AK, AR, PK, RR and KK and when X₅₁ is G then X₃₃X₄₀ is TK.
   3. Fc binding polypeptide according to item 2, wherein said Fc binding polypeptide has a lower binding affinity for the VH3 region of trastuzumab than SEQ ID NO:59.
   4. Fc binding polypeptide according to item 2 or 3, wherein X₃₃ is selected from T, S, G, Q, A, E, H, R, P, D and K, such as selected from T, S, G, Q, A, H, R, P, D and K, such as selected from T, S, G, Q, A, H, R, P and D or selected from T, S, G, Q, A, H, R, P and K; such as selected from T, S, G, Q, A, H, R and P; such as selected from T, S, G, Q, A, H and P or selected from T, G, Q, A, H, R and P; such as selected from T, G, Q, P, A and H; such as selected from T, G, Q, H and P; such as selected from T, G, Q and P or selected from G, Q, H and P or selected from T, G, H and P; such as the group selected from G and P; such is G.
   5. Fc binding polypeptide according to any one of items 2-4, wherein X₃₃ is selected from T, S, G, Q, A, E and H, such as selected from T, S, G, Q, A and E, such as selected from S, G, Q and A.
   6. Fc binding polypeptide according to any one of items 2-5, wherein X₃₃ is selected from S, G, A, E and H, such as selected from S, G, A and E.
   7. Fc binding polypeptide according to any one of items 2-6, wherein X₃₃ is selected from A, S and E.
   8. Fc binding polypeptide according to any one of items 2-7, wherein X₃₃ is selected from A and S or selected from A and E or selected from S and E.
   9. Fc binding polypeptide according to any one of items 2-8, wherein X₃₃ is S.
   10. Fc binding polypeptide according to any one of items 2-9, wherein X₄₀ is selected from E, G, R, D, K, Q, N and S or selected from E, G, R, D, K, Q, H and S or selected from G, R, D, K, Q, N, H and S, such as selected from G, R, D, K, Q, and S, such as selected from G, R, D, K and Q; such as selected from R, D, K and Q; such as selected from R, K and Q; such as selected from R and K; such as is R or is K.
   11. Fc binding polypeptide according to any one of items 2-10, wherein X₄₀ is selected from E, G, R, D, K and Q, such as selected from E, G, R, D and K or selected from E, G, R, D and Q.
   12. Fc binding polypeptide according to any one of items 2-11, wherein X₄₀ is selected from G, R, E and D.
   13. Fc binding polypeptide according to any one of items 2-12, wherein X₄₀ is selected from G, R and D.
   14. Fc binding polypeptide according to any one of items 2-11, wherein X₄₀ is selected from G, R and K.
   15. Fc binding polypeptide according to any one of items 2-14, wherein X₄₀ is selected from G and R.
   16. Fc binding polypeptide according to any one of items 2-15, wherein X₄₀ is G.
   17. Fc binding polypeptide according to any one of items 2-14, wherein X₄₀ is R.
   18. Fc binding polypeptide according to any one of items 2-17, wherein X₅₁ is selected from L, V, 1, R and S.
   19. Fc binding polypeptide according to any one of items 2-18, wherein X₅₁ is selected from L, I, R and S.
   20. Fc binding polypeptide according to any one of items 2-18, wherein X₅₁ is selected from or selected from L, V, I and R.
   21. Fc binding polypeptide according to one of items 2-18 and 20, wherein X₅₁ is selected from I, V and R; or is selected from L, I and R; or is selected from V, L and I.
   22. Fc binding polypeptide according to any one of item 2-18 and 20-21, wherein X₅₁ is selected from V and L; or is selected from V and I; or is selected from V and R; or is selected from L and I; or is selected from L and R; or is selected from I and R.
   23. Fc binding polypeptide according to any one of items 2-22, wherein X₅₁ is L or R.
   24. Fc binding polypeptide according to any one of items 2-22, wherein X₅₁ is I.
   25. Fc binding polypeptide according to any one of items 2-18 and 20-22, wherein X₅₁ is V.
   26. Fc binding polypeptide according to any one of items 2-18, wherein X₅₁ is selected from L, V, S, I and R with the proviso that when X₅₁ is L then X₃₃X₄₀ is selected from AD, HK, EG, ER, GR, AK, AR, PK, RR and KK.
   27. Fc binding polypeptide according to item 26, wherein X₅₁ is selected from L, V, S, I and R with the proviso that when X₅₁ is L then X₃₃X₄₀ are AD or HK.
   28. Fc binding polypeptide according to any one of items 2-27, wherein at least one, such as two or all three, of amino acid residues X₃₃, X₄₀ and X₅₁ is/are an uncharged amino acid residue.
   29. Fc binding polypeptide according to item 28, wherein the amino acid residue in X₃₃ is an uncharged amino acid.
   30. Fc binding polypeptide according to any one of items 28-29, wherein the amino acid residue in X₄₀ is an uncharged amino acid.
   31. Fc binding polypeptide according to any one of items 2-30, wherein at least two of the amino acid residues in positions X₃₃, X₄₀ and X₅₁ are mutated compared to SEQ ID NO:59.
   32. Fc binding polypeptide according to item 2 or 3, wherein X₃₃X₄₀X₅₁ are selected from the group consisting of ADL, AGR, AKL, ARI, ARL, DKI, DNS, DRV, EGL, ERL, GDR, GDV, GER, GGI, GGS, GNI, GQI, GRI, GRL, GRR, HKI, HKL, HQS, HRI, HSR, HSV, KEI, KKL, KRI, NHS, PDR, PHR, PHS, PKL, PKS, POR, PSI, QDI, QDV, QNS, QQI, QRI, QRV, RQR, ROS, RRI, RRL, SGV, SRR, SRS, SRV, TER, TKG, TKI, TQI, TRR and TSR, such as the group consisting of ADL, AGR, AKL, ARI, ARL, DKI, DNS, DRV, EGL, ERL, GDR, GDV, GER, GGI, GNI, GRI, GRL, GRR, HKI, HKL, HQS, HRI, HSR, HSV, KEI, KKL, KRI, NHS, PDR, PHR, PHS, PKL, PKS, POR, PSI, QDI, QDV, QNS, QQI, QRI, QRV, RQR, ROS, RRI, RRL, SGV, SRR, SRS, SRV, TER, TKI, TQI, TRR and TSR.
   33. Fc binding polypeptide according to item 32, wherein X₃₃X₄₀X₅₁ are selected from the group consisting of GGS, GGI, SRV, GRL, QRI, ARI, SGV, TER, GER, ADL, AGR, EGL, HKL, GDV, GQI and QDI or the group consisting of GGI, SRV, GRL, QRI, ARI, SGV, TER, GER, ADL, AGR, EGL, HKL, GDV and QDI.
   34. Fc binding polypeptide according to any one of items 32-33, wherein X₃₃X₄₀X₅₁ are selected from the group consisting of GGI, SRV, GRL, QRI, ARI, SGV, TER, AGR, EGL, HKL and GDV.
   35. Fc binding polypeptide according to any one of items 32-34, wherein X₃₃X₄₀X₅₁ are selected from the group consisting of GGI, ARI, SGV, AGR, EGL and HKL.
   36. Fc binding polypeptide according to any one of items 32-35, wherein X₃₃X₄₀X₅₁ are selected from the group consisting of GGI, ARI, SGV, AGR and EGL.
   37. Fc binding polypeptide according to any one of items 32-34, wherein X₃₃X₄₀X₅₁ are selected from the group consisting of GGI, SRV, GRL, QRI, ARI, SGV, TER, AGR, EGL and GDV.
   38. Fc binding polypeptide according to any one of items 32-34 and 37, wherein X₃₃X₄₀X₅₁ are selected from the group consisting of GGI, SRV, QRI, ARI, SGV, AGR and GDV.
   39. Fc binding polypeptide according to any one of items 32-33, wherein X₃₃X₄₀X₅₁ are selected from the group consisting of SRV, GRL, QRI, ARI, SGV, TER, GER, ADL, AGR, EGL, HKL, GDV, GQI and QDI.
   40. Fc binding polypeptide according to any one of items 32-34 and 37, wherein X₃₃X₄₀X₅₁ are selected from the group consisting of GGI, GRL, ARI, SGV, AGR and EGL.
   41. Fc binding polypeptide according to any one of items 32-34 and 39-40, wherein X₃₃X₄₀X₅₁ are selected from the group consisting of ARI, SGV, AGR and EGL.
   42. Fc binding polypeptide according to any one of items 32-34 and 39-41, wherein X₃₃X₄₀X₅₁ are selected from the group consisting of SGV, AGR and EGL; or the group consisting of ARI, AGR and EGL; or the group consisting of ARI, SGV and EGL; or the group consisting of ARI, SGV and AGR.
   43. Fc binding polypeptide according to any one of items 32-34 and 39-42, wherein X₃₃X₄₀X₅₁ are selected from the group consisting of ARI and SVG; or the group consisting of ARI and AGR; or the group consisting of ARI and EGL; or the group consisting of SGV and AGR; or the group consisting of SGV and EGL; or the group consisting of AGR and EGL.
   44. Fc binding polypeptide according to any one of items 32-34 and 39-42, wherein X₃₃X₄₀X₅₁ is ARI or SGV or AGR or EGL.
   45. Fc binding polypeptide according to any one of items 2-44, wherein X₃₃X₄₀X₅₁ is SGV.
   46. Fc binding polypeptide according to any one of items 2-45, wherein sequence i) corresponds to the sequence from position 24 to position 54 in a sequence selected from the group consisting of SEQ ID NO:1-57 and 208-264; such as the group consisting of SEQ ID NO:1-56 and 208-263; or such as the group consisting of SEQ ID NO:1-57, such as SEQ ID NO:1-56; or such as the group consisting of 208-264; such as SEQ ID NO:208-263.
   47. Fc binding polypeptide according to item 46, wherein sequence i) corresponds to the sequence from position 24 to position 54 in a sequence selected from the group consisting of SEQ ID NO:1-16 and 208-223; such as the group consisting of SEQ ID NO:2-14, 16, 209-221 and 223; or such as the group consisting of SEQ ID NO:1-16, such as SEQ ID NO:2-14 and 16; or such as the group consisting of 208-223; such as SEQ ID NO:209-221 and 223.
   48. Fc binding polypeptide according to any one of items 46-47, wherein sequence i) corresponds to the sequence from position 24 to position 54 in a sequence selected from the group consisting of SEQ ID NO:2, 3, 4, 5, 6, 7, 8, 11, 12, 14, 209, 210, 211, 212, 213, 214, 215, 218, 219 and 221; such as the group consisting of SEQ ID NO:2, 3, 4, 5, 6, 7, 8, 11, 12 and 14 or the group consisting of SEQ ID NO:209, 210, 211, 212, 213, 214, 215, 218, 219 and 221.
   49. Fc binding polypeptide according to any one of items 46-48, wherein sequence i) corresponds to the sequence from position 24 to position 54 in a sequence selected from the group consisting of SEQ ID NO:2, 4, 6, 7, 11, 12, 209, 211, 213, 214, 218 and 219; such as the group consisting of SEQ ID NO:2, 4, 6, 7, 11 and 12 or the group consisting of SEQ ID NO:209, 211, 213, 214, 218 and 219 or wherein sequence i) corresponds to a sequence selected from the group consisting of SEQ ID NO:2, 4, 6, 7, 12, 209, 211, 213, 214, 218 and 219; such as the group consisting of SEQ ID NO:2, 4, 6, 7 and 12 or the group consisting of SEQ ID NO:209, 211, 213, 214 and 219.
   50. Fc binding polypeptide according to any one of items 46-49, wherein sequence i) corresponds to the sequence from position 24 to position 54 in a sequence selected from the group consisting of SEQ ID NO:6, 7, 11, 12, 213, 214, 218 and 219; such as the group consisting of SEQ ID NO:6, 7, 11 and 12 or the group consisting of SEQ ID NO:213, 214, 218 and 219 or wherein sequence i) corresponds to a sequence selected from the group consisting of SEQ ID NO:6, 7, 12, 213, 214 and 219; such as the group consisting of SEQ ID NO:6, 7 and 12 or the group consisting of SEQ ID NO:213, 214 and 219.
   51. Fc binding polypeptide according to any one of items 46-50, wherein sequence i) corresponds to the sequence from position 24 to position 54 in a sequence selected from the group consisting of SEQ ID NO:6, 7, 11, 213, 214 and 218; such as the group consisting of SEQ ID NO:6, 7 and 11 or the group consisting of SEQ ID NO:213, 214 and 218 or wherein sequence i) corresponds to a sequence selected from the group consisting of SEQ ID NO:6, 7, 213 and 214; such as the group consisting of SEQ ID NO:6 and 7 or the group consisting of SEQ ID NO:213 and 214.
   52. Fc binding polypeptide according to any one of items 46-51, wherein sequence i) corresponds to the sequence from position 24 to position 54 in SEQ ID NO:7 or SEQ ID NO:214.
   53. Fc binding polypeptide according to any one of items 1-52, wherein said polypeptide derived from SpA or said Sequence A forms part of a three-helix bundle protein domain.
   54. Fc binding polypeptide according to item 53, wherein said polypeptide derived from SpA or said Sequence A forms part of two helices with an interconnecting loop, within said three-helix bundle protein domain.
   55. Fc binding polypeptide according to any one of items 53-54, wherein said three-helix bundle protein domain is selected from bacterial receptor domains.
   56. Fc binding polypeptide according to item 55, wherein said three-helix bundle protein domain is selected from domains of protein A from Staphylococcus aureus or derivatives thereof.
   57. Fc binding polypeptide according to one of items 2-56, further comprising a Sequence B arranged N-terminally of said sequence A, which Sequence B consists of an amino acid sequence selected from iv) and v), and wherein iv) and v) are defined as follows:

iv)
(SEQ ID NO: 66)
X₈X₉X₁₀X₁₁AFYX₁₅IX₁₇X₁₈X₁₉PX₂₁LX₂₃

wherein, independently from each other,
X₈ is selected from E, D and A;
X₉ is selected from Q, A, L, W, E, V, K, T and H;
X₁₀ is selected from Q and H;
X₁₁ is selected from N, A, S, E, K, H, Q, Y, T, F, L, W, I, M, V and R;
X₁₅ is selected from E, H and Q;
X₁₇ is selected from L and H;
X₁₈ is selected from H, N and K;
X₁₉ is selected from L and M;
X₂₁ is selected from N, Y and S;
X₂₃ is selected from T and N;
v) an amino acid sequence which has at least 75% identity to a sequence defined by iv).
   58. Fc binding polypeptide according to any preceding item, comprising a binding module sequence C, which Sequence C consists of Sequence A according to any one of items 2-56 and Sequence B according to item 57, in the following order from the N-terminus to the C-terminus

### [Sequence B]-[Sequence A]

or any amino acid sequence which has at least 70% identity to any sequence selected from the group consisting of: residues 8-54 in SEQ ID NO:58, residues 8-54 in SEQ ID NO:59, residues 8-54 in SEQ ID NO:60, residues 8-54 in SEQ ID NO:61, residues 8-54 in SEQ ID NO:62, residues 11-57 in SEQ ID NO:63 and residues 6-52 in SEQ ID NO:64.
59. Fc binding polypeptide in according to any preceding item, comprising a binding module Sequence C, which Sequence C consists of the sequences [H1], [L1] and [Sequence A] in the following order from the N-terminus to the C-terminus

### [H1]-[L1]-[Sequence A],

wherein [Sequence A] is as defined in any one of items 2-56,
and wherein, independently from each other, [H1] is selected from the group consisting of

(SEQ ID NO: 67)
EQQNAFYEILH;
(SEQ ID NO: 68)
DQQAAFYEILH;
(SEQ ID NO: 69)
EAQEAFYEILH;
(SEQ ID NO: 70)
DQQSAFYEILH;
(SEQ ID NO: 71)
AQQAAFYEILH;
(SEQ ID NO: 72)
EQQNAFYEILN;
(SEQ ID NO: 73)
EQQAAFYEILH;
(SEQ ID NO: 74)
AQQSAFYEILH;
(SEQ ID NO: 75)
EQQQAFYEILH;
(SEQ ID NO: 76)
EHQNAFYEILH;
(SEQ ID NO: 77)
EAQNAFYEILH;
(SEQ ID NO: 78)
ETQNAFYEILH;
(SEQ ID NO: 79)
EQQSAFYEILH;
(SEQ ID NO: 80)
EAQNAFYKILH;
(SEQ ID NO: 81)
EQQEAFYEILH;
(SEQ ID NO: 82)
EAQKAFYEILK;
(SEQ ID NO: 83)
EQHNAFYEILH;
(SEQ ID NO: 84)
EHHHALYHILH;
(SEQ ID NO: 85)
EQQKAFYAILH;
(SEQ ID NO: 86)
DQQSAFYEILN;
(SEQ ID NO: 87)
AQQNAFYQVLN;
(SEQ ID NO: 88)
EQQKAFYEILH;
(SEQ ID NO: 89)
EQQKAFYEILK;
(SEQ ID NO: 90)
EQQNAFYEILS;
(SEQ ID NO: 91)
EQQNAFYHILH;
and
(SEQ ID NO: 92)
EQQNAFYHILN;

and [L1] is selected from the group consisting of:

(SEQ ID NO: 93)
LPNLT;
(SEQ ID NO: 94)
LPNLN;
(SEQ ID NO: 95)
MPNLN;
(SEQ ID NO: 96)
LPYLT;
(SEQ ID NO: 97)
LPSLT;
(SEQ ID NO: 98)
MPNLL;
(SEQ ID NO: 99)
LPNGN;
(SEQ ID NO: 100)
LPNLS;
(SEQ ID NO: 101)
GPNAN;
(SEQ ID NO: 102)
VPNLN;
(SEQ ID NO: 103)
LPNLR;
and
(SEQ ID NO: 104)
LPNLL.

60. Fc binding polypeptide according to any one of items 58-59, comprising a binding module Sequence C selected from the group consisting of

(SEQ ID NO: 105)
EQQNAFYEILHLPNLT-[Sequence A];
(SEQ ID NO: 106)
EQQNAFYEILHLPNLN-[Sequence A];
(SEQ ID NO: 107)
DQQAAFYEILHLPNLT-[Sequence A];
(SEQ ID NO: 108)
EAQEAFYEILHLPNLT-[Sequence A];
(SEQ ID NO: 109)
DQQSAFYEILHLPNLT-[Sequence A];
(SEQ ID NO: 110)
AQQAAFYEILHLPNLT-[Sequence A];
(SEQ ID NO: 111)
EQQAAFYEILHLPNLT-[Sequence A];
(SEQ ID NO: 112)
AQQSAFYEILHLPNLT-[Sequence A];
(SEQ ID NO: 113)
EQQNAFYEILNMPNLN-[Sequence A];
(SEQ ID NO: 114)
EAQNAFYEILHLPNLT-[Sequence A];
(SEQ ID NO: 115)
EQQQAFYEILHLPNLT-[Sequence A];
(SEQ ID NO: 116)
EQQNAFYEILHLPYLT-[Sequence A];
(SEQ ID NO: 117)
ETQNAFYEILHLPNLT-[Sequence A];
(SEQ ID NO: 118)
EAQNAFYKILHLPNLT-[Sequence A];
(SEQ ID NO: 119)
EQQNAFYEILHLPNGN-[Sequence A];
(SEQ ID NO: 120)
EHQNAFYEILHLPNLN-[Sequence A];
(SEQ ID NO: 121)
EQQNAFYEILNMPNLL-[Sequence A];
(SEQ ID NO: 122)
EQQSAFYEILHLPNLT-[Sequence A];
(SEQ ID NO: 123)
EQQEAFYEILHLPNLT-[Sequence A];
(SEQ ID NO: 124)
EAQKAFYEILKLPNLT-[Sequence A];
(SEQ ID NO: 125)
EQHNAFYEILHLPNLN-[Sequence A];
(SEQ ID NO: 126)
EHHHALYHILHLPNLN-[Sequence A];
(SEQ ID NO: 127)
AQQSAFYEILHLPSLT-[Sequence A];
(SEQ ID NO: 128)
EQQKAFYAILHLPYLT-[Sequence A];
(SEQ ID NO: 129)
DQQSAFYEILNMPNLN-[Sequence A];
(SEQ ID NO: 130)
AQQNAFYQVLNMPNLN-[Sequence A];
(SEQ ID NO: 131)
EQQKAFYEILKLPNLT-[Sequence A];
(SEQ ID NO: 132)
EQQNAFYEILSLPNLT-[Sequence A];
(SEQ ID NO: 133)
EQQNAFYEILHLPNLS-[Sequence A];
(SEQ ID NO: 134)
EQQNAFYHILHLPNLN-[Sequence A];
(SEQ ID NO: 135)
EHQNAFYEILHLPNLT-[Sequence A];
(SEQ ID NO: 136)
EQQAAFYEILHLPNLN-[Sequence A];
(SEQ ID NO: 137)
AQQAAFYEILHLPSLT-[Sequence A]; wherein
[Sequence A] is as defined in any one of items
2-56.

61. Fc binding polypeptide according to any one of items 58-60, comprising a binding module Sequence C selected from the group consisting of:

vi)
(SEQ ID NO: 108)
EAQEAFYEILHLPNLT-[Sequence A]

wherein [Sequence A] is as defined in any one of items 2-56;
vii) an amino acid sequence which has at least 85% identity to the sequence defined in vi);

viii)
(SEQ ID NO: 106)
EQQNAFYEILHLPNLN-[Sequence A]

wherein [Sequence A] is as defined in any one of items 2-56; and
ix) an amino acid sequence which has at least 85% identity to the sequence defined in viii).
   62. Fc binding polypeptide according to item 61, wherein sequence vi) or viii) corresponds to the sequence from position 8 to position 54 in a sequence selected from the group consisting of SEQ ID NO:1-57 and 208-264; such as the group consisting of SEQ ID NO:1-56 and 208-263; or such as the group consisting of SEQ ID NO:1-57, such as SEQ ID NO:1-56; or such as the group consisting of 208-264; such as SEQ ID NO:208-263.
   63. Fc binding polypeptide according to any one of items 61-62, wherein sequence vi) or viii) corresponds to the sequence from position 8 to position 54 in a sequence selected from the group consisting of SEQ ID NO:1-16 and 208-223; such as the group consisting of SEQ ID NO:2-14, 16, 209-221 and 223; or such as the group consisting of SEQ ID NO:1-16, such as SEQ ID NO:2-14 and 16; or such as the group consisting of 208-223; such as SEQ ID NO:209-221 and 223.
   64. Fc binding polypeptide according to any one of items 61-63, wherein sequence vi) or viii) corresponds to the sequence from position 8 to position 54 in a sequence selected from the group consisting of SEQ ID NO:2, 3, 4, 5, 6, 7, 8, 11, 12, 14, 209, 210, 211, 212, 213, 214, 215, 218, 219 and 221; such as the group consisting of SEQ ID NO:2, 3, 4, 5, 6, 7, 8, 11, 12 and 14 or the group consisting of SEQ ID NO:209, 210, 211, 212, 213, 214, 215, 218, 219 and 221.
   65. Fc binding polypeptide according to any one of items 61-64, wherein sequence vi) or viii) corresponds to the sequence from position 8 to position 54 in a sequence selected from the group consisting of SEQ ID NO:2, 4, 6, 7, 11, 12, 209, 211, 213, 214, 218 and 219; such as the group consisting of SEQ ID NO:2, 4, 6, 7, 11 and 12 or the group consisting of SEQ ID NO:209, 211, 213, 214, 218 and 219 or wherein sequence vi) or viii) corresponds to a sequence selected from the group consisting of SEQ ID NO:2, 4, 6, 7, 12, 209, 211, 213, 214, 218 and 219; such as the group consisting of SEQ ID NO:2, 4, 6, 7 and 12 or the group consisting of SEQ ID NO:209, 211, 213, 214 and 219.
   66. Fc binding polypeptide according to any one of items 61-65, wherein sequence vi) or viii) corresponds to the sequence from position 8 to position 54 in a sequence selected from the group consisting of SEQ ID NO:6, 7, 11, 12, 213, 214, 218 and 219; such as the group consisting of SEQ ID NO:6, 7, 11 and 12 or the group consisting of SEQ ID NO:213, 214, 218 and 219 or wherein sequence vi) or viii) corresponds to a sequence selected from the group consisting of SEQ ID NO:6, 7, 12, 213, 214 and 219; such as the group consisting of SEQ ID NO:6, 7 and 12 or the group consisting of SEQ ID NO:213, 214 and 219.
   67. Fc binding polypeptide according to any one of items 61-66, wherein sequence vi) or viii) corresponds to the sequence from position 8 to position 54 in a sequence selected from the group consisting of SEQ ID NO:6, 7, 11, 213, 214 and 218; such as the group consisting of SEQ ID NO:6, 7 and 11 or the group consisting of SEQ ID NO:213, 214 and 218 or wherein sequence vi) or viii) corresponds to a sequence selected from the group consisting of SEQ ID NO:6, 7, 213 and 214; such as the group consisting of SEQ ID NO:6 and 7 or the group consisting of SEQ ID NO:213 and 214.
   68. Fc binding polypeptide according to any one of items 61-67, wherein sequence vi) or viii) corresponds to the sequence from position 8 to position 54 in SEQ ID NO:7 or SEQ ID NO:214.
   69. Fc binding polypeptide according to any preceding item, comprising a sequence [S1], [binding module Sequence C] and [S2] in the following order from the N-terminus to the C-terminus
   [S1]-[binding module Sequence C]-[S2],
   wherein [S1] or [S2] may be present or absent and [binding module sequence C] is as defined in any one of items 58-68, and
   wherein, independently from each other,
   [S1] is selected from the group consisting of

(SEQ ID NO: 138)
ADNKFNK;
(SEQ ID NO: 139)
VDAKFDK;
(SEQ ID NO: 140)
VDNKFNK;
(SEQ ID NO: 141)
IAAKHDK;
(SEQ ID NO: 142)
IAAQHDK;
(SEQ ID NO: 143)
ADNNFNK;
(SEQ ID NO: 144)
IAAKFDE;
(SEQ ID NO: 145)
PAAKHDK;
(SEQ ID NO: 146)
ADNAFNT;
FNK;
(SEQ ID NO: 147)
ADNRFNE;
(SEQ ID NO: 148)
IDSKFDE;
(SEQ ID NO: 149)
ADNRFNR;
(SEQ ID NO: 150)
ADNKHNK;
(SEQ ID NO: 151)
ADSKFDE;
(SEQ ID NO: 152)
IDAKHDE;
(SEQ ID NO: 153)
QQNKFNK;
(SEQ ID NO: 154)
ADNKFHK;
(SEQ ID NO: 155)
KFNK;
(SEQ ID NO: 156)
ADNNFNR;
(SEQ ID NO: 157)
AAAKHDK;
(SEQ ID NO: 158)
IDNKFNK;
(SEQ ID NO: 159)
IDAKFDE;
(SEQ ID NO: 160)
DNNFNK;
(SEQ ID NO: 161)
ADNKFNE;
(SEQ ID NO: 162)
AAAQHDK;
and
(SEQ ID NO: 163)
AAAKFDE;

and [S2] is selected from the group consisting of

(SEQ ID NO: 164)
QAPK;
(SEQ ID NO: 165)
QAPP;
QAP;
(SEQ ID NO: 166)
QAPR;
(SEQ ID NO: 167)
QAPE;
APK;
(SEQ ID NO: 168)
QAPG;
(SEQ ID NO: 169)
QAIK;
and
QA.

70. Fc binding polypeptide according to any preceding item, comprising a sequence selected from the group consisting of:

(SEQ ID NO: 170)
ADNKFNKEQQNAFYEILHLPNLT-[Sequence A]-QAPK;
(SEQ ID NO: 171)
ADNKFNKEQQNAFYEILHLPNLN-[Sequence A]-QAPK;
(SEQ ID NO: 172)
VDAKFDKEAQEAFYEILHLPNLT-[Sequence A]-QAPK;
(SEQ ID NO: 173)
VDNKFNKEQQNAFYEILHLPNLN-[Sequence A]-QAPK;
(SEQ ID NO: 174)
VDNKFNKEQQNAFYEILHLPNLT-[Sequence A]-QAPK;
(SEQ ID NO: 175)
PAAKHDKDQQSAFYEILHLPNLT-[Sequence A]-QAPP;
(SEQ ID NO: 176)
ADNRFNREQQNAFYEILHLPNLT-[Sequence A]-QAPR;
(SEQ ID NO: 177)
VDAKFDKEAQNAFYEILHLPNLT-[Sequence A]-QAPK;
(SEQ ID NO: 178)
ADNAFNTEQQNAFYEILHLPNLT-[Sequence A]-QAPK;
(SEQ ID NO: 179)
VDAKFDKETQNAFYEILHLPNLT-[Sequence A]-QAPK;
(SEQ ID NO: 180)
ADNKFNKEQQNAFYEILHLPNGN-[Sequence A]-QAPKA;
(SEQ ID NO: 181)
ADNKFNKEHQNAFYEILHLPNLN-[Sequence A]-QAPK;
(SEQ ID NO: 182)
FNKEQQNAFYEILHLPNLN-[Sequence A]-QAPK;
(SEQ ID NO: 183)
VDAKFDKEQQNAFYEILHLPNLT-[Sequence A]-QAPK;
(SEQ ID NO: 184)
VDAKFDKEAQKAFYEILKLPNLT-[Sequence A]-QAPK;
(SEQ ID NO: 185)
ADNKFNKEQHNAFYEILHLPNLN-[Sequence A]-QAPK;
(SEQ ID NO: 186)
VDNKFNKEAQNAFYKILHLPNLT-[Sequence A]-QAPK;
(SEQ ID NO: 187)
VDAKFDKEQQEAFYEILHLPNLT-[Sequence A]-QAPK;
(SEQ ID NO: 188)
VDAKFDKEQQKAFYEILKLPNLT-[Sequence A]-QAPK;
(SEQ ID NO: 189)
ADNKFHKEQQNAFYEILHLPNLN-[Sequence A]-QAPK;
(SEQ ID NO: 190)
ADNKFNKEQQNAFYHILHLPNLN-[Sequence A]-QAPK;
(SEQ ID NO: 191)
ADNKFNKEHQNAFYEILHLPNLT-[Sequence A]-QAPK;
(SEQ ID NO: 192)
ADNKHNKEHHHALYHILHLPNLN-[Sequence A]-QAPK;
(SEQ ID NO: 193)
KFNKEQQNAFYEILHLPNLN-[Sequence A]-QAPK;
(SEQ ID NO: 194)
ADSKFDEAQQSAFYEILHLPSLT-[Sequence A]-QAPP;
and
(SEQ ID NO: 195)
IDAKFDEAQQAAFYEILHLPNLT-[Sequence A]-QAPP,
wherein [Sequence A] is as defined in any one of
items 2-56.

71. Fc binding polypeptide according to any preceding item, comprising a sequence selected from the group consisting of:

x)
(SEQ ID NO: 172)
VDAKFDKE AQEAFYEILHLPNLT-[Sequence A]-QAPK

wherein [Sequence A] is as defined in any one of items 2-56;
xi) an amino acid sequence which has at least 86% identity to the sequence defined in x),

xvi)
(SEQ ID NO: 173)
VDNKFNKEQQNAFYEILHLPNLN-[Sequence A]-QAPK

wherein [Sequence A] is as defined in any one of items 2-56; and
xvii) an amino acid sequence which has at least 86% identity to the sequence defined in xvi).
   72. Fc binding polypeptide according to any preceding item, comprising a sequence selected from the group consisting of:

x)
(SEQ ID NO: 172)
VDAKFDKE AQEAFYEILHLPNLT-[Sequence A]-QAPK

wherein [Sequence A] is as defined in any one of items 2-56; and
xi) an amino acid sequence which has at least 86% identity to the sequence defined in x).
   73. Fc binding polypeptide according to item 71 or 72, wherein sequence x) or xvi) corresponds to a sequence selected from the group consisting of SEQ ID NO:1-57 and 208-264; such as the group consisting of SEQ ID NO:1-56 and 208-263; or such as the group consisting of SEQ ID NO:1-57, such as SEQ ID NO:1-56; or such as the group consisting of 208-264; such as SEQ ID NO:208-263.
   74. Fc binding polypeptide according to according to any one of items 71-73, wherein sequence x) or xvi) corresponds to a sequence selected from the group consisting of SEQ ID NO:1-16 and 208-223; such as the group consisting of SEQ ID NO:2-14, 16, 209-221 and 223; or such as the group consisting of SEQ ID NO:1-16, such as SEQ ID NO:2-14 and 16; or such as the group consisting of 208-223; such as SEQ ID NO:209-221 and 223.
   75. Fc binding polypeptide according to any one of items 71-74, wherein sequence x) or xvi) corresponds to a sequence selected from the group consisting of SEQ ID NO:2, 3, 4, 5, 6, 7, 8, 11, 12, 14, 209, 210, 211, 212, 213, 214, 215, 218, 219 and 221; such as the group consisting of SEQ ID NO:2, 3, 4, 5, 6, 7, 8, 11, 12 and 14 or the group consisting of SEQ ID NO:209, 210, 211, 212, 213, 214, 215, 218, 219 and 221.
   76. Fc binding polypeptide according to any one of items 71-75, wherein sequence x) or xvi) corresponds to a sequence selected from the group consisting of SEQ ID NO:2, 4, 6, 7, 11, 12, 209, 211, 213, 214, 218 and 219; such as the group consisting of SEQ ID NO:2, 4, 6, 7, 11 and 12 or the group consisting of SEQ ID NO:209, 211, 213, 214, 218 and 219 or wherein sequence x) or xvi) corresponds to a sequence selected from the group consisting of SEQ ID NO:2, 4, 6, 7, 12, 209, 211, 213, 214, 218 and 219; such as the group consisting of SEQ ID NO:2, 4, 6, 7 and 12 or the group consisting of SEQ ID NO:209, 211, 213, 214 and 219.
   77. Fc binding polypeptide according to any one of items 71-76, wherein sequence x) or xvi) corresponds to a sequence selected from the group consisting of SEQ ID NO:6, 7, 11, 12, 213, 214, 218 and 219; such as the group consisting of SEQ ID NO:6, 7, 11 and 12 or the group consisting of SEQ ID NO:213, 214, 218 and 219 or wherein sequence x) or xvi) corresponds to a sequence selected from the group consisting of SEQ ID NO:6, 7, 12, 213, 214 and 219; such as the group consisting of SEQ ID NO:6, 7 and 12 or the group consisting of SEQ ID NO:213, 214 and 219.
   78. Fc binding polypeptide according to any one of items 71-77, wherein sequence x) or xvi) corresponds to a sequence selected from the group consisting of SEQ ID NO:6, 7, 11, 213, 214 and 218; such as the group consisting of SEQ ID NO:6, 7 and 11 or the group consisting of SEQ ID NO:213, 214 and 218 or wherein sequence x) or xvi) corresponds to a sequence selected from the group consisting of SEQ ID NO:6, 7, 213 and 214; such as the group consisting of SEQ ID NO:6 and 7 or the group consisting of SEQ ID NO:213 and 214.
   79. Fc binding polypeptide according to any one of items 71-78, wherein sequence x) or xvi) corresponds to the sequence SEQ ID NO:7 or SEQ ID NO:214.
   80. Fc binding polypeptide according to any one of items 1-70, comprising a sequence selected from the group consisting of:

xii)
(SEQ ID NO: 170)
ADNKFNKEQQNAFYEILHLPNLT-[Sequence A]-QAPK

wherein [Sequence A] is as defined in any one of items 2-56; and
xiii) an amino acid sequence which has at least 86% identity to the sequence defined in xii).
   81. Fc binding polypeptide according to any one of items 1-70, comprising a sequence selected from the group consisting of:

xiv)
(SEQ ID NO: 171)
ADNKFNKEQQNAFYEILHLPNLN-[Sequence A]-QAPK

wherein [Sequence A] is as defined in any one of items 2-56; and
xv) an amino acid sequence which has at least 86% identity to the sequence defined in xiv).
   82. Fc binding polypeptide multimer, wherein each monomer of the multimer comprises a Fc binding polypeptide which is independently selected from any Fc binding polypeptide defined in any one of items 1-81.
   83. Fc binding polypeptide multimer according to item 82, wherein each monomer of the multimer comprises a Sequence A which is independently selected from any Sequence A defined in any one of items 2-56.
   84. Fc binding polypeptide multimer according to item 82 or 83, wherein said multimer is selected from the group consisting of dimer, trimer, tetramer, pentamer, hexamer, heptamer, octamer, nonamer and decamer.
   85. Fc binding polypeptide multimer according to any one of items 82-84, wherein said multimer is selected from the group consisting of tetramer, pentamer, hexamer, heptamer and octamer; such as the group consisting of pentamer, hexamer and heptamer.
   86. Fc binding polypeptide multimer according to any one of items 82-85, wherein said multimer is a hexamer.
   87. Fc binding polypeptide multimer according to any one of items 82-86, wherein said multimer is a homomer.
   88. Fc binding polypeptide multimer according to any one of items 82-86, wherein said multimer is a heteromer comprising at least two or at least three different Fc binding polypeptides each defined as in any one of items 1-81.
   89. Fc binding polypeptide multimer according to any one of items 82-86 and 88, wherein said multimer is a heteromer comprising at least two or at least three different sequence A, each defined as in any one of items 2-56
   90. Fc binding polypeptide multimer according to any one of items 82-89, further comprising at least one linker.
   91. Fc binding polypeptide multimer according to item 90, wherein said linker comprises up to 15 amino acid residues.
   92. Fc binding polypeptide according to any one of item 1-81 or a Fc binding polypeptide multimer according to any one of items 82-91 which comprises additional amino acids at the C-terminal and/or N-terminal end.
   93. Fc binding polypeptide or Fc binding polypeptide multimer according to item 92, wherein said additional amino acid(s) improve(s) production, purification, stabilization in vitro or coupling of the polypeptide.
   94. Fc binding polypeptide or Fc binding polypeptide multimer according to item 93, wherein said additional amino acid(s) improve(s) coupling of the polypeptide and are selected from the group consisting of one or more cysteine residues, a plurality of lysine residues and a plurality of histidine residues.
   95. Fc binding polypeptide multimer according to any one of items 82-94, wherein said Fc binding polypeptide monomers are expressed as a fusion protein.
   96. Fc binding polypeptide multimer according to any one of items 82-94, wherein said Fc binding polypeptide monomer units are covalently coupled together.
   97. A polynucleotide encoding an Fc binding polypeptide according to any one of items 1-81 and 92-94 or a Fc binding polypeptide multimer according to any one of items 82-95.
   98. Expression vector comprising a polynucleotide according to item 97.
   99. Host cell comprising an expression vector according to item 98.
   100. Method of producing an Fc binding polypeptide according to any one of items 1-81 and 92-94 or an Fc binding polypeptide multimer according to any one of items 82-95, comprising
culturing a host cell according to item 99 under conditions permissive of expression of said polypeptide or multimer from said expression vector, and
isolating said polypeptide or multimer.
   101. Fc binding polypeptide according to any one of items 1-81 and 92-94 or Fc binding polypeptide multimer according to any one of items 82-96, which is capable of binding to Fc such that the K_{D} value of the interaction is at most 1×10⁻⁷ M, such as at most 1×10⁻⁸ M, such as at most 1×10⁻⁹ M, such as at most 1×10⁻¹⁰ M, such as at most 1×10⁻¹¹ M.
   102. Fc binding polypeptide according to any one of items 1-82 and 92-94 and 101 or Fc binding polypeptide multimer according to any one of items 82-96 and 101, which is capable of binding to Fc such as that the K_{D} value of the interaction is at least 60%, such as at least 70%, such as at least 80%, such as at least 90% of the K_{D} value of the interaction of SEQ ID NO:58 and/or SEQ ID NO:59 with Fc.
   103. Fc binding polypeptide according to any one of items 1-82 and 92-94 and 101-102 or Fc binding polypeptide multimer according to any one of items 82-96 and 101-102, which is not capable of binding to said VH3 with a K_{D} value of the interaction of less than 1×10⁻⁴ M, such as less than 1×10⁻³ M.
   104. Fc binding polypeptide according to any one of items 1-82 and 92-94 and 101-103 or Fc binding polypeptide multimer according to any one of items 82-96 and 102-103, which exhibits has an alkali stability of at least 60%, such as at least 70%, such as at least 80%, such as at least 85%, such as at least 89%, such as at least 90%, such as at least 95%, such as at least 98%, such as at least 99% of the alkali stability of SEQ ID NO:58 and/or SEQ ID NO:59 after incubation in 0.5 M NaOH.
   105. A separation matrix comprising an Fc binding polypeptide according to any one of items 1-81 and 92-94 and 101-104 or an Fc binding polypeptide multimer according to any one of items 82-96 and 101-104 being coupled to a solid support.
   106. Separation matrix according to item 105 wherein said Fc binding polypeptide multimer is a multimer according to any one of items 82-96 and 101-104.
   107. Separation matrix according to item 106 wherein said Fc binding polypeptide multimer is a hexamer.
   108. Separation matrix according to any one of items 105-107 wherein said Fc binding polypeptide comprises a sequence selected from the group consisting of SEQ ID NO:1-57 and 208-264, such as the group consisting of SEQ ID NO:1-56 and 208-263; such as the group consisting of SEQ ID NO:1-16 and 208-223; such as the group consisting of SEQ ID NO:6, 7, 11, 12, 213, 214, 218 and 219; such as the group consisting of SEQ ID NO:6, 7, 11, 213, 214 and 218, such as the group consisting of SEQ ID NO:7 and 214;
or selected from the group consisting of
SEQ ID NO:1-57, such as selected from the group consisting of SEQ ID NO:1-56, such as the group consisting of SEQ ID NO:1-16, such as selected from the group consisting of SEQ ID NO:6, 7, 11 and 12; such as selected from the group consisting of SEQ ID NO:6, 7 and 11; such as SEQ ID NO:7.
   109. Separation matrix according to any one of items 105-107 wherein said Fc binding polypeptide multimer is a hexamer wherein each monomer of said hexamer comprises a sequence selected from the group consisting of SEQ ID NO:1-57 and 208-264; such as the group consisting of SEQ ID NO:1-56 and 208-263, such as the group consisting of SEQ ID NO:1-16 and 208-223, such as the group consisting of SEQ ID NO:6, 7, 12, 213, 214 and 219, such as the group consisting of SEQ ID NO:6, 7, 213 and 214; such as the group consisting of SEQ ID NO:7 and 214;
or selected from the group consisting of
SEQ ID NO:1-57; such as selected from the group consisting of SEQ ID NO:1-56; such as the group consisting of SEQ ID NO:1-16; such as selected from the group consisting of SEQ ID NO:6, 7 and 12; such as selected from the group consisting of SEQ ID NO:6 and 7; such as SEQ ID NO:7.
   110. Separation matrix according to any one of items 105-109, wherein the solid support comprises a polymer selected from the group consisting of polyhydroxy polymer, such as a polysaccharide; polyvinyl alcohol, a polyhydroxyalkyl acrylate, a polyhydroxyalkyl methacrylate, a polyacrylamide, and a polymethacrylamide.
   111. Separation matrix according to item 110 wherein said polymer is a polysaccharide, preferably a polysaccharide selected from dextran, starch, cellulose, pullulan, agar, and agarose.
   112. Separation matrix according to any one of items 105-111 wherein said solid support is in fibrous, beaded or particle form.
   113. Separation matrix according to any one of items 105-112, wherein said Fc binding polypeptide is coupled to the solid support by covalent or non-covalent binding.
   114. A method of isolating an immunoglobulin comprising
a) contacting a liquid sample comprising said immunoglobulin with a separation matrix according to any one of items 105-113.
   115. Method according to item 114 further comprising
b) washing said separation matrix with a washing liquid, and
c) eluting the immunoglobulin from the separation matrix with an elution liquid, and optionally
d) cleaning the separation matrix with a cleaning liquid.

1. Fc binding polypeptide derived from a Staphylococcus Protein A (SpA) or any domain thereof, wherein said polypeptide has binding affinity to an Fc region of an immunoglobulin, and has lower binding affinity for a VH3 region of trastuzumab compared to the binding affinity of SEQ ID NO:59 for the same VH3 region.

2. Fc binding polypeptide comprising a sequence A, which Sequence A consists of an amino acid sequence selected from i), ii) and iii), wherein i), ii) and iii) are defined as follows:
i)
(SEQ ID NO: 65)
EX₂₅QX₂₇X₂₈X₂₉X₃₀IX₃₂X₃₃LX₃₅X₃₆X₃₇PSX₄₀SX₄₂X₄₃X₄₄LX₄₆EAX₄₉
X₅₀X₅₁NX₅₃X₅₄
wherein, independently from each other,
X₂₅ is selected from E and D;
X₂₇ is selected from R and H;
X₂₈ is selected from N, A, S, H and W;
X₂₉ is selected from A, G and K;
X₃₀ is selected from F and A;
X₃₂ is selected from Q and H;
X₃₅ is selected from K, R and H;
X₃₆ is selected from D and H;
X₃₇ is selected from D and E;
X₄₂ is selected from A, K, L, R and T;
X₄₃ is selected from N, E, A, K and S;
X₄₄ is selected from L, I and V;
X₄₆ is selected from A, G and K;
X₄₉ is selected from K, Q and R;
X₅₀ is selected from K and R;
X₅₃ is selected from D, E and K; and
X₅₄ is selected from A and S;
ii) an amino acid sequence which has at least 83% identity to a sequence defined by i);
iii) an amino acid sequence which has at least 70% identity to any sequence selected from the group consisting of: residues 24-54 in SEQ ID NO:58, residues 24-54 in SEQ ID NO:59, residues 24-54 in SEQ ID NO:60, residues 24-54 in SEQ ID NO:61, residues 24-54 in SEQ ID NO:62, residues 27-57 in SEQ ID NO:63 and residues 22-52 in SEQ ID NO:64,
wherein additionally, in each of i), ii) and iii) independently from each other,
X₃₃ is selected from T, S, G, Q, A, E, H, R, P, D, K and N;
X₄₀ is selected from E, G, R, D, K, Q, N, H and S; and
X₅₁ is selected from L, V, S, I, R and G;
with the proviso that when X₅₁ is L then X₃₃X₄₀ is selected from AD, HK, EG, ER, GR, AK, AR, PK, RR and KK and when X₅₁ is G then X₃₃X₄₀ is TK.

3. Fc binding polypeptide according to claim 2, wherein said Fc binding polypeptide has a lower binding affinity for the VH3 region of trastuzumab, than SEQ ID NO:59.

4. Fc binding polypeptide according to claim 2, wherein X₃₃ is selected from T, S, G, Q, A, E and H; and/or X₄₀ is selected from E, G, R, D, K and Q; and/or X₅₁ is selected from L, V, S, I and R.

5. Fc binding polypeptide according to claim 2, wherein X₃₃X₄₀X₅₁ are selected from the group consisting of ADL, AGR, AKL, ARI, ARL, DKI, DNS, DRV, EGL, ERL, GDR, GDV, GER, GGI, GGS, GNI, GQI, GRI, GRL, GRR, HKI, HKL, HQS, HRI, HSR, HSV, KEI, KKL, KRI, NHS, PDR, PHR, PHS, PKL, PKS, PQR, PSI, QDI, QDV, QNS, QQI, QRI, QRV, RQR, RQS, RRI, RRL, SGV, SRR, SRS, SRV, TER, TKG, TKI, TQI, TRR and TSR, such as the group consisting of ADL, AGR, AKL, ARI, ARL, DKI, DNS, DRV, EGL, ERL, GDR, GDV, GER, GGI, GNI, GRI, GRL, GRR, HKI, HKL, HQS, HRI, HSR, HSV, KEI, KKL, KRI, NHS, PDR, PHR, PHS, PKL, PKS, PQR, PSI, QDI, QDV, QNS, QQI, QRI, QRV, RQR, RQS, RRI, RRL, SGV, SRR, SRS, SRV, TER, TKI, TQI, TRR and TSR.

6. Fc binding polypeptide according to claim 2, wherein X₃₃X₄₀X₅₁ are selected from the group consisting of GGS, GGI, SRV, GRL, QRI, ARI, SGV, TER, GER, ADL, AGR, EGL, HKL, GDV, GQI and QDI, such as the group consisting of ARI, SGV, AGR and EGL.

7. Fc binding polypeptide according to claim 2, wherein X₃₃X₄₀X₅₁ is SGV.

8. Fc binding polypeptide according claim 2, further comprising a Sequence B arranged N-terminally of said sequence A, which Sequence B consists of an amino acid sequence selected from iv) and v), and wherein iv) and v) are defined as follows:
iv)
(SEQ ID NO: 66)
X₈X₉X₁₀X₁₁AFYX₁₅IX₁₇X₁₈X₁₉PX₂₁LX₂₃
wherein, independently from each other,
X₈ is selected from E, D and A;
X₉ is selected from Q, A, L, W, E, V, K, T and H;
X₁₀ is selected from Q and H;
X₁₁ is selected from N, A, S, E, K, H, Q, Y, T, F, L, W, I, M, V and R;
X₁₅ is selected from E, H and Q;
X₁₇ is selected from L and H;
X₁₈ is selected from H, N and K;
X₁₉ is selected from L and M;
X₂₁ is selected from N, Y and S;
X₂₃ is selected from T and N;
v) an amino acid sequence which has at least 75% identity to a sequence defined by iv).

9. Fc binding polypeptide according to claim 1, comprising a binding module Sequence C,
which Sequence C consists of Sequence A and Sequence B, in the following order from the N-terminus to the C-terminus
[Sequence B]-[Sequence A]
or any amino acid sequence which has at least 70% identity to any sequence selected from the group consisting of: residues 8-54 in SEQ ID NO:58, residues 8-54 in SEQ ID NO:59, residues 8-54 in SEQ ID NO:60, residues 8-54 in SEQ ID NO:61, residues 8-54 in SEQ ID NO:62, residues 11-57 in SEQ ID NO:63 and residues 6-52 in SEQ ID NO:64.

10. Fc binding polypeptide according to claim 2, comprising a sequence selected from the group consisting of:
x)
(SEQ ID NO: 172)
VDAKFDKE AQEAFYEILHLPNLT-[Sequence A]-QAPK
wherein [Sequence A] is as defined in claim 2;
xi) an amino acid sequence which has at least 86% identity to the sequence defined in x);
xvi)
(SEQ ID NO: 173)
VDNKFNKEQQNAFYEILHLPNLN-[Sequence A]-QAPK
wherein [Sequence A] is as defined in claim 2; and
xvii) an amino acid sequence which has at least 86% identity to the sequence defined in xvi).

11. Fc binding polypeptide according to claim 10, wherein sequence x) or xvi) corresponds to a sequence selected from the group consisting of SEQ ID NO:1-57, 208-264, 269-305 such as the group consisting of SEQ ID NO:1-16 and 208-223, such as corresponds to SEQ ID NO:7 or 214.

12. Fc binding polypeptide according to claim 10, wherein sequence x) corresponds to a sequence selected from the group consisting of SEQ ID NO:1-57, such as the group consisting of SEQ ID NO:1-16, such as corresponds to SEQ ID NO:7.

13. Fc binding polypeptide multimer, wherein each monomer of the multimer comprises a Fc binding polypeptide which is independently selected from any Fc binding polypeptide as defined in claim 1 and wherein the multimer preferably is a hexamer.

14. Fc binding polypeptide according to claim 1 or Fc binding polypeptide multimer thereof, which is capable of binding to Fc such that the K_{D} value of the interaction is at most 1×10⁻⁷ M, such as at most 1×10⁻⁸ M, such as at most 1×10⁻⁹ M, such as at most 1×10⁻¹⁰ M, such as at most 1×10⁻¹¹ M.

15. Fc binding polypeptide according to claim 1, or Fc binding polypeptide multimer thereof, which is not capable of binding to said VH3 with a K_{D} value of the interaction of less than 1×10⁻⁴ M, such as less than 1×10⁻³ M.

16. A separation matrix comprising an Fc binding polypeptide according to claim 1, or an Fc binding polypeptide multimer thereof being coupled to a solid support, which solid support preferably is in fibrous, beaded or particle form.

17. A method of isolating an immunoglobulin comprising
a) contacting a liquid sample comprising said immunoglobulin with a separation matrix according claim 16.


SEQUENCE LISTING (305 sequences)

SEQ ID NO: 1 - protein, 58 aa, synthetic construct
  REGION 1..58: note = Engineered Fc binding polypeptide
VDAKFDKEAQEAFYEILHLPNLTEEQRNAFIQGLKDDPSGSKAILAEAKKSNDAQAPK

SEQ ID NO: 2 - protein, 58 aa, synthetic construct
  REGION 1..58: note = Engineered Fc binding polypeptide
VDAKFDKEAQEAFYEILHLPNLTEEQRNAFIQGLKDDPSGSKAILAEAKKINDAQAPK

SEQ ID NO: 3 - protein, 58 aa, synthetic construct
  REGION 1..58: note = Engineered Fc binding polypeptide
VDAKFDKEAQEAFYEILHLPNLTEEQRNAFIQSLKDDPSRSKAILAEAKKVNDAQAPK

SEQ ID NO: 4 - protein, 58 aa, synthetic construct
  REGION 1..58: note = Engineered Fc binding polypeptide
VDAKFDKEAQEAFYEILHLPNLTEEQRNAFIQGLKDDPSRSKAILAEAKKLNDAQAPK

SEQ ID NO: 5 - protein, 58 aa, synthetic construct
  REGION 1..58: note = Engineered Fc binding polypeptide
VDAKFDKEAQEAFYEILHLPNLTEEQRNAFIQQLKDDPSRSKAILAEAKKINDAQAPK

SEQ ID NO: 6 - protein, 58 aa, synthetic construct
  REGION 1..58: note = Engineered Fc binding polypeptide
VDAKFDKEAQEAFYEILHLPNLTEEQRNAFIQALKDDPSRSKAILAEAKKINDAQAPK

SEQ ID NO: 7 - protein, 58 aa, synthetic construct
  REGION 1..58: note = Engineered Fc binding polypeptide
VDAKFDKEAQEAFYEILHLPNLTEEQRNAFIQSLKDDPSGSKAILAEAKKVNDAQAPK

SEQ ID NO: 8 - protein, 58 aa, synthetic construct
  REGION 1..58: note = Engineered Fc binding polypeptide
VDAKFDKEAQEAFYEILHLPNLTEEQRNAFIQTLKDDPSESKAILAEAKKRNDAQAPK

SEQ ID NO: 9 - protein, 58 aa, synthetic construct
  REGION 1..58: note = Engineered Fc binding polypeptide
VDAKFDKEAQEAFYEILHLPNLTEEQRNAFIQGLKDDPSESKAILAEAKKRNDAQAPK

SEQ ID NO: 10 - protein, 58 aa, synthetic construct
  REGION 1..58: note = Engineered Fc binding polypeptide
VDAKFDKEAQEAFYEILHLPNLTEEQRNAFIQALKDDPSDSKAILAEAKKLNDAQAPK

SEQ ID NO: 11 - protein, 58 aa, synthetic construct
  REGION 1..58: note = Engineered Fc binding polypeptide
VDAKFDKEAQEAFYEILHLPNLTEEQRNAFIQALKDDPSGSKAILAEAKKRNDAQAPK

SEQ ID NO: 12 - protein, 58 aa, synthetic construct
  REGION 1..58: note = Engineered Fc binding polypeptide
VDAKFDKEAQEAFYEILHLPNLTEEQRNAFIQELKDDPSGSKAILAEAKKLNDAQAPK

SEQ ID NO: 13 - protein, 58 aa, synthetic construct
  REGION 1..58: note = Engineered Fc binding polypeptide
VDAKFDKEAQEAFYEILHLPNLTEEQRNAFIQHLKDDPSKSKAILAEAKKLNDAQAPK

SEQ ID NO: 14 - protein, 58 aa, synthetic construct
  REGION 1..58: note = Engineered Fc binding polypeptide
VDAKFDKEAQEAFYEILHLPNLTEEQRNAFIQGLKDDPSDSKAILAEAKKVNDAQAPK

SEQ ID NO: 15 - protein, 58 aa, synthetic construct
  REGION 1..58: note = Engineered Fc binding polypeptide
VDAKFDKEAQEAFYEILHLPNLTEEQRNAFIQGLKDDPSQSKAILAEAKKINDAQAPK

SEQ ID NO: 16 - protein, 58 aa, synthetic construct
  REGION 1..58: note = Engineered Fc binding polypeptide
VDAKFDKEAQEAFYEILHLPNLTEEQRNAFIQQLKDDPSDSKAILAEAKKINDAQAPK

SEQ ID NO: 17 - protein, 58 aa, synthetic construct
  REGION 1..58: note = Engineered Fc binding polypeptide
VDAKFDKEAQEAFYEILHLPNLTEEQRNAFIQALKDDPSKSKAILAEAKKLNDAQAPK

SEQ ID NO: 18 - protein, 58 aa, synthetic construct
  REGION 1..58: note = Engineered Fc binding polypeptide
VDAKFDKEAQEAFYEILHLPNLTEEQRNAFIQALKDDPSRSKAILAEAKKLNDAQAPK

SEQ ID NO: 19 - protein, 58 aa, synthetic construct
  REGION 1..58: note = Engineered Fc binding polypeptide
VDAKFDKEAQEAFYEILHLPNLTEEQRNAFIQDLKDDPSKSKAILAEAKKINDAQAPK

SEQ ID NO: 20 - protein, 58 aa, synthetic construct
  REGION 1..58: note = Engineered Fc binding polypeptide
VDAKFDKEAQEAFYEILHLPNLTEEQRNAFIQDLKDDPSNSKAILAEAKKSNDAQAPK

SEQ ID NO: 21 - protein, 58 aa, synthetic construct
  REGION 1..58: note = Engineered Fc binding polypeptide
VDAKFDKEAQEAFYEILHLPNLTEEQRNAFIQDLKDDPSRSKAILAEAKKVNDAQAPK

SEQ ID NO: 22 - protein, 58 aa, synthetic construct
  REGION 1..58: note = Engineered Fc binding polypeptide
VDAKFDKEAQEAFYEILHLPNLTEEQRNAFIQELKDDPSRSKAILAEAKKLNDAQAPK

SEQ ID NO: 23 - protein, 58 aa, synthetic construct
  REGION 1..58: note = Engineered Fc binding polypeptide
VDAKFDKEAQEAFYEILHLPNLTEEQRNAFIQGLKDDPSDSKAILAEAKKRNDAQAPK

SEQ ID NO: 24 - protein, 58 aa, synthetic construct
  REGION 1..58: note = Engineered Fc binding polypeptide
VDAKFDKEAQEAFYEILHLPNLTEEQRNAFIQGLKDDPSNSKAILAEAKKINDAQAPK

SEQ ID NO: 25 - protein, 58 aa, synthetic construct
  REGION 1..58: note = Engineered Fc binding polypeptide
VDAKFDKEAQEAFYEILHLPNLTEEQRNAFIQGLKDDPSRSKAILAEAKKINDAQAPK

SEQ ID NO: 26 - protein, 58 aa, synthetic construct
  REGION 1..58: note = Engineered Fc binding polypeptide
VDAKFDKEAQEAFYEILHLPNLTEEQRNAFIQGLKDDPSRSKAILAEAKKRNDAQAPK

SEQ ID NO: 27 - protein, 58 aa, synthetic construct
  REGION 1..58: note = Engineered Fc binding polypeptide
VDAKFDKEAQEAFYEILHLPNLTEEQRNAFIQHLKDDPSKSKAILAEAKKINDAQAPK

SEQ ID NO: 28 - protein, 58 aa, synthetic construct
  REGION 1..58: note = Engineered Fc binding polypeptide
VDAKFDKEAQEAFYEILHLPNLTEEQRNAFIQHLKDDPSQSKAILAEAKKSNDAQAPK

SEQ ID NO: 29 - protein, 58 aa, synthetic construct
  REGION 1..58: note = Engineered Fc binding polypeptide
VDAKFDKEAQEAFYEILHLPNLTEEQRNAFIQHLKDDPSRSKAILAEAKKINDAQAPK

SEQ ID NO: 30 - protein, 58 aa, synthetic construct
  REGION 1..58: note = Engineered Fc binding polypeptide
VDAKFDKEAQEAFYEILHLPNLTEEQRNAFIQHLKDDPSSSKAILAEAKKRNDAQAPK

SEQ ID NO: 31 - protein, 58 aa, synthetic construct
  REGION 1..58: note = Engineered Fc binding polypeptide
VDAKFDKEAQEAFYEILHLPNLTEEQRNAFIQHLKDDPSSSKAILAEAKKVNDAQAPK

SEQ ID NO: 32 - protein, 58 aa, synthetic construct
  REGION 1..58: note = Engineered Fc binding polypeptide
VDAKFDKEAQEAFYEILHLPNLTEEQRNAFIQKLKDDPSESKAILAEAKKINDAQAPK

SEQ ID NO: 33 - protein, 58 aa, synthetic construct
  REGION 1..58: note = Engineered Fc binding polypeptide
VDAKFDKEAQEAFYEILHLPNLTEEQRNAFIQKLKDDPSKSKAILAEAKKLNDAQAPK

SEQ ID NO: 34 - protein, 58 aa, synthetic construct
  REGION 1..58: note = Engineered Fc binding polypeptide
VDAKFDKEAQEAFYEILHLPNLTEEQRNAFIQKLKDDPSRSKAILAEAKKINDAQAPK

SEQ ID NO: 35 - protein, 58 aa, synthetic construct
  REGION 1..58: note = Engineered Fc binding polypeptide
VDAKFDKEAQEAFYEILHLPNLTEEQRNAFIQNLKDDPSHSKAILAEAKKSNDAQAPK

SEQ ID NO: 36 - protein, 58 aa, synthetic construct
  REGION 1..58: note = Engineered Fc binding polypeptide
VDAKFDKEAQEAFYEILHLPNLTEEQRNAFIQPLKDDPSDSKAILAEAKKRNDAQAPK

SEQ ID NO: 37 - protein, 58 aa, synthetic construct
  REGION 1..58: note = Engineered Fc binding polypeptide
VDAKFDKEAQEAFYEILHLPNLTEEQRNAFIQPLKDDPSHSKAILAEAKKRNDAQAPK

SEQ ID NO: 38 - protein, 58 aa, synthetic construct
  REGION 1..58: note = Engineered Fc binding polypeptide
VDAKFDKEAQEAFYEILHLPNLTEEQRNAFIQPLKDDPSHSKAILAEAKKSNDAQAPK

SEQ ID NO: 39 - protein, 58 aa, synthetic construct
  REGION 1..58: note = Engineered Fc binding polypeptide
VDAKFDKEAQEAFYEILHLPNLTEEQRNAFIQPLKDDPSKSKAILAEAKKLNDAQAPK

SEQ ID NO: 40 - protein, 58 aa, synthetic construct
  REGION 1..58: note = Engineered Fc binding polypeptide
VDAKFDKEAQEAFYEILHLPNLTEEQRNAFIQPLKDDPSKSKAILAEAKKSNDAQAPK

SEQ ID NO: 41 - protein, 58 aa, synthetic construct
  REGION 1..58: note = Engineered Fc binding polypeptide
VDAKFDKEAQEAFYEILHLPNLTEEQRNAFIQPLKDDPSQSKAILAEAKKRNDAQAPK

SEQ ID NO: 42 - protein, 58 aa, synthetic construct
  REGION 1..58: note = Engineered Fc binding polypeptide
VDAKFDKEAQEAFYEILHLPNLTEEQRNAFIQPLKDDPSSSKAILAEAKKINDAQAPK

SEQ ID NO: 43 - protein, 58 aa, synthetic construct
  REGION 1..58: note = Engineered Fc binding polypeptide
VDAKFDKEAQEAFYEILHLPNLTEEQRNAFIQQLKDDPSDSKAILAEAKKVNDAQAPK

SEQ ID NO: 44 - protein, 58 aa, synthetic construct
  REGION 1..58: note = Engineered Fc binding polypeptide
VDAKFDKEAQEAFYEILHLPNLTEEQRNAFIQQLKDDPSNSKAILAEAKKSNDAQAPK

SEQ ID NO: 45 - protein, 58 aa, synthetic construct
  REGION 1..58: note = Engineered Fc binding polypeptide
VDAKFDKEAQEAFYEILHLPNLTEEQRNAFIQQLKDDPSQSKAILAEAKKINDAQAPK

SEQ ID NO: 46 - protein, 58 aa, synthetic construct
  REGION 1..58: note = Engineered Fc binding polypeptide
VDAKFDKEAQEAFYEILHLPNLTEEQRNAFIQQLKDDPSRSKAILAEAKKVNDAQAPK

SEQ ID NO: 47 - protein, 58 aa, synthetic construct
  REGION 1..58: note = Engineered Fc binding polypeptide
VDAKFDKEAQEAFYEILHLPNLTEEQRNAFIQRLKDDPSQSKAILAEAKKRNDAQAPK

SEQ ID NO: 48 - protein, 58 aa, synthetic construct
  REGION 1..58: note = Engineered Fc binding polypeptide
VDAKFDKEAQEAFYEILHLPNLTEEQRNAFIQRLKDDPSQSKAILAEAKKSNDAQAPK

SEQ ID NO: 49 - protein, 58 aa, synthetic construct
  REGION 1..58: note = Engineered Fc binding polypeptide
VDAKFDKEAQEAFYEILHLPNLTEEQRNAFIQRLKDDPSRSKAILAEAKKINDAQAPK

SEQ ID NO: 50 - protein, 58 aa, synthetic construct
  REGION 1..58: note = Engineered Fc binding polypeptide
VDAKFDKEAQEAFYEILHLPNLTEEQRNAFIQRLKDDPSRSKAILAEAKKLNDAQAPK

SEQ ID NO: 51 - protein, 58 aa, synthetic construct
  REGION 1..58: note = Engineered Fc binding polypeptide
VDAKFDKEAQEAFYEILHLPNLTEEQRNAFIQSLKDDPSRSKAILAEAKKRNDAQAPK

SEQ ID NO: 52 - protein, 58 aa, synthetic construct
  REGION 1..58: note = Engineered Fc binding polypeptide
VDAKFDKEAQEAFYEILHLPNLTEEQRNAFIQSLKDDPSRSKAILAEAKKSNDAQAPK

SEQ ID NO: 53 - protein, 58 aa, synthetic construct
  REGION 1..58: note = Engineered Fc binding polypeptide
VDAKFDKEAQEAFYEILHLPNLTEEQRNAFIQTLKDDPSKSKAILAEAKKINDAQAPK

SEQ ID NO: 54 - protein, 58 aa, synthetic construct
  REGION 1..58: note = Engineered Fc binding polypeptide
VDAKFDKEAQEAFYEILHLPNLTEEQRNAFIQTLKDDPSQSKAILAEAKKINDAQAPK

SEQ ID NO: 55 - protein, 58 aa, synthetic construct
  REGION 1..58: note = Engineered Fc binding polypeptide
VDAKFDKEAQEAFYEILHLPNLTEEQRNAFIQTLKDDPSRSKAILAEAKKRNDAQAPK

SEQ ID NO: 56 - protein, 58 aa, synthetic construct
  REGION 1..58: note = Engineered Fc binding polypeptide
VDAKFDKEAQEAFYEILHLPNLTEEQRNAFIQTLKDDPSSSKAILAEAKKRNDAQAPK

SEQ ID NO: 57 - protein, 58 aa, synthetic construct
  REGION 1..58: note = Engineered Fc binding polypeptide
VDAKFDKEAQEAFYEILHLPNLTEEQRNAFIQTLKDDPSKSKAILAEAKKGNDAQAPK

SEQ ID NO: 58 - protein, 58 aa, synthetic construct
  REGION 1..58: note = Engineered Fc binding polypeptide
VDAKFDKEAQEAFYEILHLPNLTEEQRNAFIQSLKDDPSVSKAILAEAKKLNDAQAPK

SEQ ID NO: 59 - protein, 58 aa, synthetic construct [sequence omitted]

SEQ ID NO: 60 - protein, 58 aa, Staphylococcus aureus
ADNKFNKEQQNAFYEILHLPNLTEEQRNGFIQSLKDDPSVSKEILAEAKKLNDAQAPK

SEQ ID NO: 61 - protein, 58 aa, Staphylococcus aureus [sequence omitted]

SEQ ID NO: 62 - protein, 58 aa, Staphylococcus aureus
ADNNFNKEQQNAFYEILNMPNLNEEQRNGFIQSLKDDPSQSANLLAEAKKLNESQAPK

SEQ ID NO: 63 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 64 - protein, 56 aa, Staphylococcus aureus
AQHDEAQQNAFYQVLNMPNLNADQRNGFIQSLKDDPSQSANVLGEAQKLNDSQAPK

SEQ ID NO: 65 - protein, 31 aa, synthetic construct
  REGION 1..31: note = Engineered Fc binding polypeptide
  SITE 2: note = misc_feature - Xaa can be any naturally occurring amino acid
  REGION 4..7: note = misc_feature - Xaa can be any naturally occurring amino acid
  REGION 9..10: note = misc_feature - Xaa can be any naturally occurring amino acid
  REGION 12..14: note = misc_feature - Xaa can be any naturally occurring amino acid
  SITE 17: note = misc_feature - Xaa can be any naturally occurring amino acid
  REGION 19..21: note = misc_feature - Xaa can be any naturally occurring amino acid
  SITE 23: note = misc_feature - Xaa can be any naturally occurring amino acid
  REGION 26..28: note = misc_feature - Xaa can be any naturally occurring amino acid
  REGION 30..31: note = misc_feature - Xaa can be any naturally occurring amino acid
EXQXXXXIXXLXXXPSXSXXXLXEAXXXNXX

SEQ ID NO: 66 - protein, 16 aa, synthetic construct
  REGION 1..16: note = Engineered Fc binding polypeptide
  REGION 1..4: note = misc_feature - Xaa can be any naturally occurring amino acid
  SITE 8: note = misc_feature - Xaa can be any naturally occurring amino acid
  REGION 10..12: note = misc_feature - Xaa can be any naturally occurring amino acid
  SITE 14: note = misc_feature - Xaa can be any naturally occurring amino acid
  SITE 16: note = misc_feature - Xaa can be any naturally occurring amino acid
XXXXAFYXIXXXPXLX

SEQ ID NO: 67 - protein, 11 aa, synthetic construct
  REGION 1..11: note = Engineered Fc binding polypeptide
EQQNAFYEILH

SEQ ID NO: 68 - protein, 11 aa, synthetic construct
  REGION 1..11: note = Engineered Fc binding polypeptide
DQQAAFYEILH

SEQ ID NO: 69 - protein, 11 aa, synthetic construct
  REGION 1..11: note = Engineered Fc binding polypeptide
EAQEAFYEILH

SEQ ID NO: 70 - protein, 11 aa, synthetic construct
  REGION 1..11: note = Engineered Fc binding polypeptide
DQQSAFYEILH

SEQ ID NO: 71 - protein, 11 aa, synthetic construct
  REGION 1..11: note = Engineered Fc binding polypeptide
AQQAAFYEILH

SEQ ID NO: 72 - protein, 11 aa, synthetic construct
  REGION 1..11: note = Engineered Fc binding polypeptide
EQQNAFYEILN

SEQ ID NO: 73 - protein, 11 aa, synthetic construct
  REGION 1..11: note = Engineered Fc binding polypeptide
EQQAAFYEILH

SEQ ID NO: 74 - protein, 11 aa, synthetic construct
  REGION 1..11: note = Engineered Fc binding polypeptide
AQQSAFYEILH

SEQ ID NO: 75 - protein, 11 aa, synthetic construct
  REGION 1..11: note = Engineered Fc binding polypeptide
EQQQAFYEILH

SEQ ID NO: 76 - protein, 11 aa, synthetic construct
  REGION 1..11: note = Engineered Fc binding polypeptide
EHQNAFYEILH

SEQ ID NO: 77 - protein, 11 aa, synthetic construct
  REGION 1..11: note = Engineered Fc binding polypeptide
EAQNAFYEILH

SEQ ID NO: 78 - protein, 11 aa, synthetic construct
  REGION 1..11: note = Engineered Fc binding polypeptide
ETQNAFYEILH

SEQ ID NO: 79 - protein, 11 aa, synthetic construct
  REGION 1..11: note = Engineered Fc binding polypeptide
EQQSAFYEILH

SEQ ID NO: 80 - protein, 11 aa, synthetic construct
  REGION 1..11: note = Engineered Fc binding polypeptide
EAQNAFYKILH

SEQ ID NO: 81 - protein, 11 aa, synthetic construct
  REGION 1..11: note = Engineered Fc binding polypeptide
EQQEAFYEILH

SEQ ID NO: 82 - protein, 11 aa, synthetic construct
  REGION 1..11: note = Engineered Fc binding polypeptide
EAQKAFYEILK

SEQ ID NO: 83 - protein, 11 aa, synthetic construct
  REGION 1..11: note = Engineered Fc binding polypeptide
EQHNAFYEILH

SEQ ID NO: 84 - protein, 11 aa, synthetic construct
  REGION 1..11: note = Engineered Fc binding polypeptide
EHHHALYHILH

SEQ ID NO: 85 - protein, 11 aa, synthetic construct
  REGION 1..11: note = Engineered Fc binding polypeptide
EQQKAFYAILH

SEQ ID NO: 86 - protein, 11 aa, synthetic construct
  REGION 1..11: note = Engineered Fc binding polypeptide
DQQSAFYEILN

SEQ ID NO: 87 - protein, 11 aa, synthetic construct
  REGION 1..11: note = Engineered Fc binding polypeptide
AQQNAFYQVLN

SEQ ID NO: 88 - protein, 11 aa, synthetic construct
  REGION 1..11: note = Engineered Fc binding polypeptide
EQQKAFYEILH

SEQ ID NO: 89 - protein, 11 aa, synthetic construct
  REGION 1..11: note = Engineered Fc binding polypeptide
EQQKAFYEILK

SEQ ID NO: 90 - protein, 11 aa, synthetic construct
  REGION 1..11: note = Engineered Fc binding polypeptide
EQQNAFYEILS

SEQ ID NO: 91 - protein, 11 aa, synthetic construct
  REGION 1..11: note = Engineered Fc binding polypeptide
EQQNAFYHILH

SEQ ID NO: 92 - protein, 11 aa, synthetic construct
  REGION 1..11: note = Engineered Fc binding polypeptide
EQQNAFYHILN

SEQ ID NO: 93 - protein, 5 aa, synthetic construct
  REGION 1..5: note = Engineered Fc binding polypeptide
LPNLT

SEQ ID NO: 94 - protein, 5 aa, synthetic construct
  REGION 1..5: note = Engineered Fc binding polypeptide
LPNLN

SEQ ID NO: 95 - protein, 5 aa, synthetic construct
  REGION 1..5: note = Engineered Fc binding polypeptide
MPNLN

SEQ ID NO: 96 - protein, 5 aa, synthetic construct
  REGION 1..5: note = Engineered Fc binding polypeptide
LPYLT

SEQ ID NO: 97 - protein, 5 aa, synthetic construct
  REGION 1..5: note = Engineered Fc binding polypeptide
LPSLT

SEQ ID NO: 98 - protein, 5 aa, synthetic construct
  REGION 1..5: note = Engineered Fc binding polypeptide
MPNLL

SEQ ID NO: 99 - protein, 5 aa, synthetic construct
  REGION 1..5: note = Engineered Fc binding polypeptide
LPNGN

SEQ ID NO: 100 - protein, 5 aa, synthetic construct
  REGION 1..5: note = Engineered Fc binding polypeptide
LPNLS

SEQ ID NO: 101 - protein, 5 aa, synthetic construct
  REGION 1..5: note = Engineered Fc binding polypeptide
GPNAN

SEQ ID NO: 102 - protein, 5 aa, synthetic construct
  REGION 1..5: note = Engineered Fc binding polypeptide
VPNLN

SEQ ID NO: 103 - protein, 5 aa, synthetic construct
  REGION 1..5: note = Engineered Fc binding polypeptide
LPNLR

SEQ ID NO: 104 - protein, 5 aa, synthetic construct
  REGION 1..5: note = Engineered Fc binding polypeptide
LPNLL

SEQ ID NO: 105 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILHLPNLTEXQXXXXIXXLXXXPSXSXXXLXEAXXXNXX

SEQ ID NO: 106 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILHLPNLNEXQXXXXIXXLXXXPSXSXXXLXEAXXXNXX

SEQ ID NO: 107 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
DQQAAFYEILHLPNLTEXQXXXXIXXLXXXPSXSXXXLXEAXXXNXX

SEQ ID NO: 108 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EAQEAFYEILHLPNLTEXQXXXXIXXLXXXPSXSXXXLXEAXXXNXX

SEQ ID NO: 109 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
DQQSAFYEILHLPNLTEXQXXXXIXXLXXXPSXSXXXLXEAXXXNXX

SEQ ID NO: 110 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
AQQAAFYEILHLPNLTEXQXXXXIXXLXXXPSXSXXXLXEAXXXNXX

SEQ ID NO: 111 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQAAFYEILHLPNLTEXQXXXXIXXLXXXPSXSXXXLXEAXXXNXX

SEQ ID NO: 112 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
AQQSAFYEILHLPNLTEXQXXXXIXXLXXXPSXSXXXLXEAXXXNXX

SEQ ID NO: 113 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILNMPNLNEXQXXXXIXXLXXXPSXSXXXLXEAXXXNXX

SEQ ID NO: 114 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EAQNAFYEILHLPNLTEXQXXXXIXXLXXXPSXSXXXLXEAXXXNXX

SEQ ID NO: 115 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQQAFYEILHLPNLTEXQXXXXIXXLXXXPSXSXXXLXEAXXXNXX

SEQ ID NO: 116 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILHLPYLTEXQXXXXIXXLXXXPSXSXXXLXEAXXXNXX

SEQ ID NO: 117 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
ETQNAFYEILHLPNLTEXQXXXXIXXLXXXPSXSXXXLXEAXXXNXX

SEQ ID NO: 118 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EAQNAFYKILHLPNLTEXQXXXXIXXLXXXPSXSXXXLXEAXXXNXX

SEQ ID NO: 119 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILHLPNGNEXQXXXXIXXLXXXPSXSXXXLXEAXXXNXX

SEQ ID NO: 120 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EHQNAFYEILHLPNLNEXQXXXXIXXLXXXPSXSXXXLXEAXXXNXX

SEQ ID NO: 121 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILNMPNLLEXQXXXXIXXLXXXPSXSXXXLXEAXXXNXX

SEQ ID NO: 122 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQSAFYEILHLPNLTEXQXXXXIXXLXXXPSXSXXXLXEAXXXNXX

SEQ ID NO: 123 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQEAFYEILHLPNLTEXQXXXXIXXLXXXPSXSXXXLXEAXXXNXX

SEQ ID NO: 124 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EAQKAFYEILKLPNLTEXQXXXXIXXLXXXPSXSXXXLXEAXXXNXX

SEQ ID NO: 125 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQHNAFYEILHLPNLNEXQXXXXIXXLXXXPSXSXXXLXEAXXXNXX

SEQ ID NO: 126 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EHHHALYHILHLPNLNEXQXXXXIXXLXXXPSXSXXXLXEAXXXNXX

SEQ ID NO: 127 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
AQQSAFYEILHLPSLTEXQXXXXIXXLXXXPSXSXXXLXEAXXXNXX

SEQ ID NO: 128 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQKAFYAILHLPYLTEXQXXXXIXXLXXXPSXSXXXLXEAXXXNXX

SEQ ID NO: 129 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
DQQSAFYEILNMPNLNEXQXXXXIXXLXXXPSXSXXXLXEAXXXNXX

SEQ ID NO: 130 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
AQQNAFYQVLNMPNLNEXQXXXXIXXLXXXPSXSXXXLXEAXXXNXX

SEQ ID NO: 131 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQKAFYEILKLPNLTEXQXXXXIXXLXXXPSXSXXXLXEAXXXNXX

SEQ ID NO: 132 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILSLPNLTEXQXXXXIXXLXXXPSXSXXXLXEAXXXNXX

SEQ ID NO: 133 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYEILHLPNLSEXQXXXXIXXLXXXPSXSXXXLXEAXXXNXX

SEQ ID NO: 134 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQNAFYHILHLPNLNEXQXXXXIXXLXXXPSXSXXXLXEAXXXNXX

SEQ ID NO: 135 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EHQNAFYEILHLPNLTEXQXXXXIXXLXXXPSXSXXXLXEAXXXNXX

SEQ ID NO: 136 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
EQQAAFYEILHLPNLNEXQXXXXIXXLXXXPSXSXXXLXEAXXXNXX

SEQ ID NO: 137 - protein, 47 aa, synthetic construct
  REGION 1..47: note = Engineered Fc binding polypeptide
  SITE 18: note = misc_feature - Xaa can be any naturally occurring amino acid
  REGION 20..23: note = misc_feature - Xaa can be any naturally occurring amino acid
  REGION 25..26: note = misc_feature - Xaa can be any naturally occurring amino acid
  REGION 28..30: note = misc_feature - Xaa can be any naturally occurring amino acid
  SITE 33: note = misc_feature - Xaa can be any naturally occurring amino acid
  REGION 35..37: note = misc_feature - Xaa can be any naturally occurring amino acid
  SITE 39: note = misc_feature - Xaa can be any naturally occurring amino acid
  REGION 42..44: note = misc_feature - Xaa can be any naturally occurring amino acid
  REGION 46..47: note = misc_feature - Xaa can be any naturally occurring amino acid
AQQAAFYEILHLPSLTEXQXXXXIXXLXXXPSXSXXXLXEAXXXNXX

SEQ ID NO: 138 - protein, 7 aa, synthetic construct
  REGION 1..7: note = Engineered Fc binding polypeptide
ADNKFNK

SEQ ID NO: 139 - protein, 7 aa, synthetic construct
  REGION 1..7: note = Engineered Fc binding polypeptide
VDAKFDK

SEQ ID NO: 140 - protein, 7 aa, synthetic construct
  REGION 1..7: note = Engineered Fc binding polypeptide
VDNKFNK

SEQ ID NO: 141 - protein, 7 aa, synthetic construct
  REGION 1..7: note = Engineered Fc binding polypeptide
IAAKHDK

SEQ ID NO: 142 - protein, 7 aa, synthetic construct
  REGION 1..7: note = Engineered Fc binding polypeptide
IAAQHDK

SEQ ID NO: 143 - protein, 7 aa, synthetic construct
  REGION 1..7: note = Engineered Fc binding polypeptide
ADNNFNK

SEQ ID NO: 144 - protein, 7 aa, synthetic construct
  REGION 1..7: note = Engineered Fc binding polypeptide
IAAKFDE

SEQ ID NO: 145 - protein, 7 aa, synthetic construct
  REGION 1..7: note = Engineered Fc binding polypeptide
PAAKHDK

SEQ ID NO: 146 - protein, 7 aa, synthetic construct
  REGION 1..7: note = Engineered Fc binding polypeptide
ADNAFNT

SEQ ID NO: 147 - protein, 7 aa, synthetic construct
  REGION 1..7: note = Engineered Fc binding polypeptide
ADNRFNE

SEQ ID NO: 148 - protein, 7 aa, synthetic construct
  REGION 1..7: note = Engineered Fc binding polypeptide
IDSKFDE

SEQ ID NO: 149 - protein, 7 aa, synthetic construct
  REGION 1..7: note = Engineered Fc binding polypeptide
ADNRFNR

SEQ ID NO: 150 - protein, 7 aa, synthetic construct
  REGION 1..7: note = Engineered Fc binding polypeptide
ADNKHNK

SEQ ID NO: 151 - protein, 7 aa, synthetic construct
  REGION 1..7: note = Engineered Fc binding polypeptide
ADSKFDE

SEQ ID NO: 152 - protein, 7 aa, synthetic construct
  REGION 1..7: note = Engineered Fc binding polypeptide
IDAKHDE

SEQ ID NO: 153 - protein, 7 aa, synthetic construct
  REGION 1..7: note = Engineered Fc binding polypeptide
QQNKFNK

SEQ ID NO: 154 - protein, 7 aa, synthetic construct
  REGION 1..7: note = Engineered Fc binding polypeptide
ADNKFHK

SEQ ID NO: 155 - protein, 4 aa, synthetic construct
  REGION 1..4: note = Engineered Fc binding polypeptide
KFNK

SEQ ID NO: 156 - protein, 7 aa, synthetic construct
  REGION 1..7: note = Engineered Fc binding polypeptide
ADNNFNR

SEQ ID NO: 157 - protein, 7 aa, synthetic construct
  REGION 1..7: note = Engineered Fc binding polypeptide
AAAKHDK

SEQ ID NO: 158 - protein, 7 aa, synthetic construct
  REGION 1..7: note = Engineered Fc binding polypeptide
IDNKFNK

SEQ ID NO: 159 - protein, 7 aa, synthetic construct
  REGION 1..7: note = Engineered Fc binding polypeptide
IDAKFDE

SEQ ID NO: 160 - protein, 6 aa, synthetic construct
  REGION 1..6: note = Engineered Fc binding polypeptide
DNNFNK

SEQ ID NO: 161 - protein, 7 aa, synthetic construct
  REGION 1..7: note = Engineered Fc binding polypeptide
ADNKFNE

SEQ ID NO: 162 - protein, 7 aa, synthetic construct
  REGION 1..7: note = Engineered Fc binding polypeptide
AAAQHDK

SEQ ID NO: 163 - protein, 7 aa, synthetic construct
  REGION 1..7: note = Engineered Fc binding polypeptide
AAAKFDE

SEQ ID NO: 164 - protein, 4 aa, synthetic construct
  REGION 1..4: note = Engineered Fc binding polypeptide
QAPK

SEQ ID NO: 165 - protein, 4 aa, synthetic construct
  REGION 1..4: note = Engineered Fc binding polypeptide
QAPP

SEQ ID NO: 166 - protein, 4 aa, synthetic construct
  REGION 1..4: note = Engineered Fc binding polypeptide
QAPR

SEQ ID NO: 167 - protein, 4 aa, synthetic construct
  REGION 1..4: note = Engineered Fc binding polypeptide
QAPE

SEQ ID NO: 168 - protein, 4 aa, synthetic construct
  REGION 1..4: note = Engineered Fc binding polypeptide
QAPG

SEQ ID NO: 169 - protein, 4 aa, synthetic construct
  REGION 1..4: note = Engineered Fc binding polypeptide
QAIK

SEQ ID NO: 170 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEQQNAFYEILHLPNLTEXQXXXXIXXLXXXPSXSXXXLXEAXXXNXXQAPK

SEQ ID NO: 171 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEQQNAFYEILHLPNLNEXQXXXXIXXLXXXPSXSXXXLXEAXXXNXXQAPK

SEQ ID NO: 172 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKEAQEAFYEILHLPNLTEXQXXXXIXXLXXXPSXSXXXLXEAXXXNXXQAPK

SEQ ID NO: 173 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDNKFNKEQQNAFYEILHLPNLNEXQXXXXIXXLXXXPSXSXXXLXEAXXXNXXQAPK

SEQ ID NO: 174 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDNKFNKEQQNAFYEILHLPNLTEXQXXXXIXXLXXXPSXSXXXLXEAXXXNXXQAPK

SEQ ID NO: 175 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
PAAKHDKDQQSAFYEILHLPNLTEXQXXXXIXXLXXXPSXSXXXLXEAXXXNXXQAPP

SEQ ID NO: 176 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNRFNREQQNAFYEILHLPNLTEXQXXXXIXXLXXXPSXSXXXLXEAXXXNXXQAPR

SEQ ID NO: 177 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKEAQNAFYEILHLPNLTEXQXXXXIXXLXXXPSXSXXXLXEAXXXNXXQAPK

SEQ ID NO: 178 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNAFNTEQQNAFYEILHLPNLTEXQXXXXIXXLXXXPSXSXXXLXEAXXXNXXQAPK

SEQ ID NO: 179 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKETQNAFYEILHLPNLTEXQXXXXIXXLXXXPSXSXXXLXEAXXXNXXQAPK

SEQ ID NO: 180 - protein, 59 aa, synthetic construct
  REGION 1..59: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEQQNAFYEILHLPNGNEXQXXXXIXXLXXXPSXSXXXLXEAXXXNXXQAPKA

SEQ ID NO: 181 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEHQNAFYEILHLPNLNEXQXXXXIXXLXXXPSXSXXXLXEAXXXNXXQAPK

SEQ ID NO: 182 - protein, 54 aa, synthetic construct
  REGION 1..54: note = Engineered Fc binding polypeptide
  SITE 21: note = misc_feature - Xaa can be any naturally occurring amino acid
  REGION 23..26: note = misc_feature - Xaa can be any naturally occurring amino acid
  REGION 28..29: note = misc_feature - Xaa can be any naturally occurring amino acid
  REGION 31..33: note = misc_feature - Xaa can be any naturally occurring amino acid
  SITE 36: note = misc_feature - Xaa can be any naturally occurring amino acid
  REGION 38..40: note = misc_feature - Xaa can be any naturally occurring amino acid
  SITE 42: note = misc_feature - Xaa can be any naturally occurring amino acid
  REGION 45..47: note = misc_feature - Xaa can be any naturally occurring amino acid
  REGION 49..50: note = misc_feature - Xaa can be any naturally occurring amino acid
FNKEQQNAFYEILHLPNLNEXQXXXXIXXLXXXPSXSXXXLXEAXXXNXXQAPK

SEQ ID NO: 183 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKEQQNAFYEILHLPNLTEXQXXXXIXXLXXXPSXSXXXLXEAXXXNXXQAPK

SEQ ID NO: 184 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKEAQKAFYEILKLPNLTEXQXXXXIXXLXXXPSXSXXXLXEAXXXNXXQAPK

SEQ ID NO: 185 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEQHNAFYEILHLPNLNEXQXXXXIXXLXXXPSXSXXXLXEAXXXNXXQAPK

SEQ ID NO: 186 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDNKFNKEAQNAFYKILHLPNLTEXQXXXXIXXLXXXPSXSXXXLXEAXXXNXXQAPK

SEQ ID NO: 187 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKEQQEAFYEILHLPNLTEXQXXXXIXXLXXXPSXSXXXLXEAXXXNXXQAPK

SEQ ID NO: 188 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
VDAKFDKEQQKAFYEILKLPNLTEXQXXXXIXXLXXXPSXSXXXLXEAXXXNXXQAPK

SEQ ID NO: 189 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFHKEQQNAFYEILHLPNLNEXQXXXXIXXLXXXPSXSXXXLXEAXXXNXXQAPK

SEQ ID NO: 190 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEQQNAFYHILHLPNLNEXQXXXXIXXLXXXPSXSXXXLXEAXXXNXXQAPK

SEQ ID NO: 191 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKFNKEHQNAFYEILHLPNLTEXQXXXXIXXLXXXPSXSXXXLXEAXXXNXXQAPK

SEQ ID NO: 192 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADNKHNKEHHHALYHILHLPNLNEXQXXXXIXXLXXXPSXSXXXLXEAXXXNXXQAPK

SEQ ID NO: 193 - protein, 55 aa, synthetic construct
  REGION 1..55: note = Engineered Fc binding polypeptide
  SITE 22: note = misc_feature - Xaa can be any naturally occurring amino acid
  REGION 24..27: note = misc_feature - Xaa can be any naturally occurring amino acid
  REGION 29..30: note = misc_feature - Xaa can be any naturally occurring amino acid
  REGION 32..34: note = misc_feature - Xaa can be any naturally occurring amino acid
  SITE 37: note = misc_feature - Xaa can be any naturally occurring amino acid
  REGION 39..41: note = misc_feature - Xaa can be any naturally occurring amino acid
  SITE 43: note = misc_feature - Xaa can be any naturally occurring amino acid
  REGION 46..48: note = misc_feature - Xaa can be any naturally occurring amino acid
  REGION 50..51: note = misc_feature - Xaa can be any naturally occurring amino acid
KFNKEQQNAFYEILHLPNLNEXQXXXXIXXLXXXPSXSXXXLXEAXXXNXXQAPK

SEQ ID NO: 194 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
ADSKFDEAQQSAFYEILHLPSLTEXQXXXXIXXLXXXPSXSXXXLXEAXXXNXXQAPP

SEQ ID NO: 195 - protein, 58 aa, synthetic construct
  REGION 1..58: note = Engineered Fc binding polypeptide
  SITE 25: note = misc_feature - Xaa can be any naturally occurring amino acid
  REGION 27..30: note = misc_feature - Xaa can be any naturally occurring amino acid
  REGION 32..33: note = misc_feature - Xaa can be any naturally occurring amino acid
  REGION 35..37: note = misc_feature - Xaa can be any naturally occurring amino acid
  SITE 40: note = misc_feature - Xaa can be any naturally occurring amino acid
  REGION 42..44: note = misc_feature - Xaa can be any naturally occurring amino acid
  SITE 46: note = misc_feature - Xaa can be any naturally occurring amino acid
  REGION 49..51: note = misc_feature - Xaa can be any naturally occurring amino acid
  REGION 53..54: note = misc_feature - Xaa can be any naturally occurring amino acid
IDAKFDEAQQAAFYEILHLPNLTEXQXXXXIXXLXXXPSXSXXXLXEAXXXNXXQAPP

SEQ ID NO: 196 - protein, 214 aa, synthetic construct
  REGION 1..214: note = Humanized mus musculus antibody fragment
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 450 aa, synthetic construct
  REGION 1..450: note = Humanized mus musculus antibody fragment
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 214 aa, synthetic construct; identical to SEQ ID NO: 196
  REGION 1..214: note = Humanized mus musculus antibody fragment

SEQ ID NO: 199 - protein, 237 aa, synthetic construct
  REGION 1..237: note = Humanized mus musculus antibody fragment
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEL

SEQ ID NO: 200 - protein, 213 aa, synthetic construct
  REGION 1..213: note = Humanized mus musculus antibody fragment
LGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 46 aa, Streptococcus sp.
LAEAKVLANRELDKYGVSDYYKNLINNAKTVEGVKALIDEILAALP

SEQ ID NO: 202 - protein, 110 aa, synthetic construct
  REGION 1..110: note = Engineered Fc binding polypeptide
VDNKFNKEQQNAFYEILHLPNLNEEQRNAFIQSLKDDPSQSANLLAEAKKLNDAQAPKASGGGSLAEAKVLANRELDKYGVSDYYKNLINNAKTVEGVKALIDEILAALP

SEQ ID NO: 203 - protein, 349 aa, synthetic construct
  REGION 1..349: note = Engineered Fc binding polypeptide
VDAKFDKEAQEAFYEILHLPNLTEEQRNAFIQALKDDPSRSKAILAEAKKINDAQAPKVDAKFDKEAQEAFYEILHLPNLTEEQRNAFIQALKDDPSRSKAILAEAKKINDAQAPKVDAKFDKEAQEAFYEILHLPNLTEEQRNAFIQALKDDPSRSKAILAEAKKINDAQAPKVDAKFDKEAQEAFYEILHLPNLTEEQRNAFIQALKDDPSRSKAILAEAKKINDAQAPKVDAKFDKEAQEAFYEILHLPNLTEEQRNAFIQALKDDPSRSKAILAEAKKINDAQAPKVDAKFDKEAQEAFYEILHLPNLTEEQRNAFIQALKDDPSRSKAILAEAKKINDAQAPKC

SEQ ID NO: 204 - protein, 349 aa, synthetic construct
  REGION 1..349: note = Engineered Fc binding polypeptide
VDAKFDKEAQEAFYEILHLPNLTEEQRNAFIQSLKDDPSGSKAILAEAKKVNDAQAPKVDAKFDKEAQEAFYEILHLPNLTEEQRNAFIQSLKDDPSGSKAILAEAKKVNDAQAPKVDAKFDKEAQEAFYEILHLPNLTEEQRNAFIQSLKDDPSGSKAILAEAKKVNDAQAPKVDAKFDKEAQEAFYEILHLPNLTEEQRNAFIQSLKDDPSGSKAILAEAKKVNDAQAPKVDAKFDKEAQEAFYEILHLPNLTEEQRNAFIQSLKDDPSGSKAILAEAKKVNDAQAPKVDAKFDKEAQEAFYEILHLPNLTEEQRNAFIQSLKDDPSGSKAILAEAKKVNDAQAPKC

SEQ ID NO: 205 - protein, 349 aa, synthetic construct
  REGION 1..349: note = Engineered Fc binding polypeptide
VDAKFDKEAQEAFYEILHLPNLTEEQRNAFIQELKDDPSGSKAILAEAKKLNDAQAPKVDAKFDKEAQEAFYEILHLPNLTEEQRNAFIQELKDDPSGSKAILAEAKKLNDAQAPKVDAKFDKEAQEAFYEILHLPNLTEEQRNAFIQELKDDPSGSKAILAEAKKLNDAQAPKVDAKFDKEAQEAFYEILHLPNLTEEQRNAFIQELKDDPSGSKAILAEAKKLNDAQAPKVDAKFDKEAQEAFYEILHLPNLTEEQRNAFIQELKDDPSGSKAILAEAKKLNDAQAPKVDAKFDKEAQEAFYEILHLPNLTEEQRNAFIQELKDDPSGSKAILAEAKKLNDAQAPKC

SEQ ID NO: 206 - protein, 349 aa, synthetic construct
  REGION 1..349: note = Engineered Fc binding polypeptide
VDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKC

SEQ ID NO: 207 - protein, 349 aa, synthetic construct
  REGION 1..349: note = Engineered Fc binding polypeptide
  SITE 33: note = misc_feature - Xaa can be any naturally occurring amino acid
  SITE 40: note = misc_feature - Xaa can be any naturally occurring amino acid
  SITE 51: note = misc_feature - Xaa can be any naturally occurring amino acid
  SITE 91: note = misc_feature - Xaa can be any naturally occurring amino acid
  SITE 98: note = misc_feature - Xaa can be any naturally occurring amino acid
  SITE 109: note = misc_feature - Xaa can be any naturally occurring amino acid
  SITE 149: note = misc_feature - Xaa can be any naturally occurring amino acid
  SITE 156: note = misc_feature - Xaa can be any naturally occurring amino acid
  SITE 167: note = misc_feature - Xaa can be any naturally occurring amino acid
  SITE 207: note = misc_feature - Xaa can be any naturally occurring amino acid
  SITE 214: note = misc_feature - Xaa can be any naturally occurring amino acid
  SITE 225: note = misc_feature - Xaa can be any naturally occurring amino acid
  SITE 265: note = misc_feature - Xaa can be any naturally occurring amino acid
  SITE 272: note = misc_feature - Xaa can be any naturally occurring amino acid
  SITE 283: note = misc_feature - Xaa can be any naturally occurring amino acid
  SITE 323: note = misc_feature - Xaa can be any naturally occurring amino acid
  SITE 330: note = misc_feature - Xaa can be any naturally occurring amino acid
  SITE 341: note = misc_feature - Xaa can be any naturally occurring amino acid
VDAKFDKEAQEAFYEILHLPNLTEEQRNAFIQXLKDDPSXSKAILAEAKKXNDAQAPKVDAKFDKEAQEAFYEILHLPNLTEEQRNAFIQXLKDDPSXSKAILAEAKKXNDAQAPKVDAKFDKEAQEAFYEILHLPNLTEEQRNAFIQXLKDDPSXSKAILAEAKKXNDAQAPKVDAKFDKEAQEAFYEILHLPNLTEEQRNAFIQXLKDDPSXSKAILAEAKKXNDAQAPKVDAKFDKEAQEAFYEILHLPNLTEEQRNAFIQXLKDDPSXSKAILAEAKKXNDAQAPKVDAKFDKEAQEAFYEILHLPNLTEEQRNAFIQXLKDDPSXSKAILAEAKKXNDAQAPKC

SEQ ID NO: 208 - protein, 58 aa, synthetic construct
VDNKFNKEQQNAFYEILHLPNLNEEQRNAFIQGLKDDPSGSANLLAEAKKSNDAQAPK

SEQ ID NO: 209 - protein, 58 aa, synthetic construct
VDNKFNKEQQNAFYEILHLPNLNEEQRNAFIQGLKDDPSGSANLLAEAKKINDAQAPK

SEQ ID NO: 210 - protein, 58 aa, synthetic construct
VDNKFNKEQQNAFYEILHLPNLNEEQRNAFIQSLKDDPSRSANLLAEAKKVNDAQAPK

SEQ ID NO: 211 - protein, 58 aa, synthetic construct
VDNKFNKEQQNAFYEILHLPNLNEEQRNAFIQGLKDDPSRSANLLAEAKKLNDAQAPK

SEQ ID NO: 212 - protein, 58 aa, synthetic construct
VDNKFNKEQQNAFYEILHLPNLNEEQRNAFIQQLKDDPSRSANLLAEAKKINDAQAPK

SEQ ID NO: 213 - protein, 58 aa, synthetic construct
VDNKFNKEQQNAFYEILHLPNLNEEQRNAFIQALKDDPSRSANLLAEAKKINDAQAPK

SEQ ID NO: 214 - protein, 58 aa, synthetic construct
VDNKFNKEQQNAFYEILHLPNLNEEQRNAFIQSLKDDPSGSANLLAEAKKVNDAQAPK

SEQ ID NO: 215 - protein, 58 aa, synthetic construct
VDNKFNKEQQNAFYEILHLPNLNEEQRNAFIQTLKDDPSESANLLAEAKKRNDAQAPK

SEQ ID NO: 216 - protein, 58 aa, synthetic construct
VDNKFNKEQQNAFYEILHLPNLNEEQRNAFIQGLKDDPSESANLLAEAKKRNDAQAPK

SEQ ID NO: 217 - protein, 58 aa, synthetic construct
VDNKFNKEQQNAFYEILHLPNLNEEQRNAFIQALKDDPSDSANLLAEAKKLNDAQAPK

SEQ ID NO: 218 - protein, 58 aa, synthetic construct
VDNKFNKEQQNAFYEILHLPNLNEEQRNAFIQALKDDPSGSANLLAEAKKRNDAQAPK

SEQ ID NO: 219 - protein, 58 aa, synthetic construct
VDNKFNKEQQNAFYEILHLPNLNEEQRNAFIQELKDDPSGSANLLAEAKKLNDAQAPK

SEQ ID NO: 220 - protein, 58 aa, synthetic construct
VDNKFNKEQQNAFYEILHLPNLNEEQRNAFIQHLKDDPSKSANLLAEAKKLNDAQAPK

SEQ ID NO: 221 - protein, 58 aa, synthetic construct
VDNKFNKEQQNAFYEILHLPNLNEEQRNAFIQGLKDDPSDSANLLAEAKKVNDAQAPK

SEQ ID NO: 222 - protein, 58 aa, synthetic construct
VDNKFNKEQQNAFYEILHLPNLNEEQRNAFIQGLKDDPSQSANLLAEAKKINDAQAPK

SEQ ID NO: 223 - protein, 58 aa, synthetic construct
VDNKFNKEQQNAFYEILHLPNLNEEQRNAFIQQLKDDPSDSANLLAEAKKINDAQAPK

SEQ ID NO: 224 - protein, 58 aa, synthetic construct
VDNKFNKEQQNAFYEILHLPNLNEEQRNAFIQALKDDPSKSANLLAEAKKLNDAQAPK

SEQ ID NO: 225 - protein, 58 aa, synthetic construct
VDNKFNKEQQNAFYEILHLPNLNEEQRNAFIQALKDDPSRSANLLAEAKKLNDAQAPK

SEQ ID NO: 226 - protein, 58 aa, synthetic construct
VDNKFNKEQQNAFYEILHLPNLNEEQRNAFIQDLKDDPSKSANLLAEAKKINDAQAPK

SEQ ID NO: 227 - protein, 58 aa, synthetic construct
VDNKFNKEQQNAFYEILHLPNLNEEQRNAFIQDLKDDPSNSANLLAEAKKSNDAQAPK

SEQ ID NO: 228 - protein, 58 aa, synthetic construct
VDNKFNKEQQNAFYEILHLPNLNEEQRNAFIQDLKDDPSRSANLLAEAKKVNDAQAPK

SEQ ID NO: 229 - protein, 58 aa, synthetic construct
VDNKFNKEQQNAFYEILHLPNLNEEQRNAFIQELKDDPSRSANLLAEAKKLNDAQAPK

SEQ ID NO: 230 - protein, 58 aa, synthetic construct
VDNKFNKEQQNAFYEILHLPNLNEEQRNAFIQGLKDDPSDSANLLAEAKKRNDAQAPK

SEQ ID NO: 231 - protein, 58 aa, synthetic construct
VDNKFNKEQQNAFYEILHLPNLNEEQRNAFIQGLKDDPSNSANLLAEAKKINDAQAPK

SEQ ID NO: 232 - protein, 58 aa, synthetic construct
VDNKFNKEQQNAFYEILHLPNLNEEQRNAFIQGLKDDPSRSANLLAEAKKINDAQAPK

SEQ ID NO: 233 - protein, 58 aa, synthetic construct
VDNKFNKEQQNAFYEILHLPNLNEEQRNAFIQGLKDDPSRSANLLAEAKKRNDAQAPK

SEQ ID NO: 234 - protein, 58 aa, synthetic construct
VDNKFNKEQQNAFYEILHLPNLNEEQRNAFIQHLKDDPSKSANLLAEAKKINDAQAPK

SEQ ID NO: 235 - protein, 58 aa, synthetic construct
VDNKFNKEQQNAFYEILHLPNLNEEQRNAFIQHLKDDPSQSANLLAEAKKSNDAQAPK

SEQ ID NO: 236 - protein, 58 aa, synthetic construct
VDNKFNKEQQNAFYEILHLPNLNEEQRNAFIQHLKDDPSRSANLLAEAKKINDAQAPK

SEQ ID NO: 237 - protein, 58 aa, synthetic construct
VDNKFNKEQQNAFYEILHLPNLNEEQRNAFIQHLKDDPSSSANLLAEAKKRNDAQAPK

SEQ ID NO: 238 - protein, 58 aa, synthetic construct
VDNKFNKEQQNAFYEILHLPNLNEEQRNAFIQHLKDDPSSSANLLAEAKKVNDAQAPK

SEQ ID NO: 239 - protein, 58 aa, synthetic construct
VDNKFNKEQQNAFYEILHLPNLNEEQRNAFIQKLKDDPSESANLLAEAKKINDAQAPK

SEQ ID NO: 240 - protein, 58 aa, synthetic construct
VDNKFNKEQQNAFYEILHLPNLNEEQRNAFIQKLKDDPSKSANLLAEAKKLNDAQAPK

SEQ ID NO: 241 - protein, 58 aa, synthetic construct
VDNKFNKEQQNAFYEILHLPNLNEEQRNAFIQKLKDDPSRSANLLAEAKKINDAQAPK

SEQ ID NO: 242 - protein, 58 aa, synthetic construct
VDNKFNKEQQNAFYEILHLPNLNEEQRNAFIQNLKDDPSHSANLLAEAKKSNDAQAPK

SEQ ID NO: 243 - protein, 58 aa, synthetic construct
VDNKFNKEQQNAFYEILHLPNLNEEQRNAFIQPLKDDPSDSANLLAEAKKRNDAQAPK

SEQ ID NO: 244 - protein, 58 aa, synthetic construct
VDNKFNKEQQNAFYEILHLPNLNEEQRNAFIQPLKDDPSHSANLLAEAKKRNDAQAPK

SEQ ID NO: 245 - protein, 58 aa, synthetic construct
VDNKFNKEQQNAFYEILHLPNLNEEQRNAFIQPLKDDPSHSANLLAEAKKSNDAQAPK

SEQ ID NO: 246 - protein, 58 aa, synthetic construct
VDNKFNKEQQNAFYEILHLPNLNEEQRNAFIQPLKDDPSKSANLLAEAKKLNDAQAPK

SEQ ID NO: 247 - protein, 58 aa, synthetic construct
VDNKFNKEQQNAFYEILHLPNLNEEQRNAFIQPLKDDPSKSANLLAEAKKSNDAQAPK

SEQ ID NO: 248 - protein, 58 aa, synthetic construct
VDNKFNKEQQNAFYEILHLPNLNEEQRNAFIQPLKDDPSQSANLLAEAKKRNDAQAPK

SEQ ID NO: 249 - protein, 58 aa, synthetic construct
VDNKFNKEQQNAFYEILHLPNLNEEQRNAFIQPLKDDPSSSANLLAEAKKINDAQAPK

SEQ ID NO: 250 - protein, 58 aa, synthetic construct
VDNKFNKEQQNAFYEILHLPNLNEEQRNAFIQQLKDDPSDSANLLAEAKKVNDAQAPK

SEQ ID NO: 251 - protein, 58 aa, synthetic construct
VDNKFNKEQQNAFYEILHLPNLNEEQRNAFIQQLKDDPSNSANLLAEAKKSNDAQAPK

SEQ ID NO: 252 - protein, 58 aa, synthetic construct
VDNKFNKEQQNAFYEILHLPNLNEEQRNAFIQQLKDDPSQSANLLAEAKKINDAQAPK

SEQ ID NO: 253 - protein, 58 aa, synthetic construct
VDNKFNKEQQNAFYEILHLPNLNEEQRNAFIQQLKDDPSRSANLLAEAKKVNDAQAPK

SEQ ID NO: 254 - protein, 58 aa, synthetic construct
VDNKFNKEQQNAFYEILHLPNLNEEQRNAFIQRLKDDPSQSANLLAEAKKRNDAQAPK

SEQ ID NO: 255 - protein, 58 aa, synthetic construct
VDNKFNKEQQNAFYEILHLPNLNEEQRNAFIQRLKDDPSQSANLLAEAKKSNDAQAPK

SEQ ID NO: 256 - protein, 58 aa, synthetic construct
VDNKFNKEQQNAFYEILHLPNLNEEQRNAFIQRLKDDPSRSANLLAEAKKINDAQAPK

SEQ ID NO: 257 - protein, 58 aa, synthetic construct
VDNKFNKEQQNAFYEILHLPNLNEEQRNAFIQRLKDDPSRSANLLAEAKKLNDAQAPK

SEQ ID NO: 258 - protein, 58 aa, synthetic construct
VDNKFNKEQQNAFYEILHLPNLNEEQRNAFIQSLKDDPSRSANLLAEAKKRNDAQAPK

SEQ ID NO: 259 - protein, 58 aa, synthetic construct
VDNKFNKEQQNAFYEILHLPNLNEEQRNAFIQSLKDDPSRSANLLAEAKKSNDAQAPK

SEQ ID NO: 260 - protein, 58 aa, synthetic construct
VDNKFNKEQQNAFYEILHLPNLNEEQRNAFIQTLKDDPSKSANLLAEAKKINDAQAPK

SEQ ID NO: 261 - protein, 58 aa, synthetic construct
VDNKFNKEQQNAFYEILHLPNLNEEQRNAFIQTLKDDPSQSANLLAEAKKINDAQAPK

SEQ ID NO: 262 - protein, 58 aa, synthetic construct
VDNKFNKEQQNAFYEILHLPNLNEEQRNAFIQTLKDDPSRSANLLAEAKKRNDAQAPK

SEQ ID NO: 263 - protein, 58 aa, synthetic construct
VDNKFNKEQQNAFYEILHLPNLNEEQRNAFIQTLKDDPSSSANLLAEAKKRNDAQAPK

SEQ ID NO: 264 - protein, 58 aa, synthetic construct
VDNKFNKEQQNAFYEILHLPNLNEEQRNAFIQTLKDDPSKSANLLAEAKKGNDAQAPK

SEQ ID NO: 265 - protein, 58 aa, synthetic construct
IAAQHDKDQQAAFYEILHLPNLTEEQRNAFIQSLRDDPSQSLEILAEAKKLNDAQAPK

SEQ ID NO: 266 - protein, 58 aa, synthetic construct
IAAQHDKEHQAAFYEILHLPNLTEDQRNAFIQSLRDDPSQSLEILGEAKKLNDAQAPK

SEQ ID NO: 267 - protein, 58 aa, synthetic construct
VDNKFNKEQQNAFTEILHLPNLTEEQRNAFIQSLKDDPSQTKEILAEAKKLNDAQAPK

SEQ ID NO: 268 - protein, 58 aa, synthetic construct [sequence omitted]

SEQ ID NO: 269 - protein, 58 aa, synthetic construct
IAAQHDKDQQAAFYEILHLPNLTEEQRNAFIQALRDDPSRSLEILAEAKKINDAQAPK

SEQ ID NO: 270 - protein, 58 aa, synthetic construct
IAAQHDKEHQAAFYEILHLPNLTEDQRNAFIQALRDDPSRSLEILGEAKKINDAQAPK

SEQ ID NO: 271 - protein, 58 aa, synthetic construct
VDNKFNKEQQNAFTEILHLPNLTEEQRNAFIQALKDDPSRTKEILAEAKKINDAQAPK

SEQ ID NO: 272 - protein, 56 aa, synthetic construct
AQHDEAQQNAFYQVLNMPNLNADQRNGFIQALKDDPSRSANVLGEAQKINDSQAPK

SEQ ID NO: 273 - protein, 61 aa, synthetic construct
ADAQQNKFNKDQQSAFYEILNMPNLNEEQRNGFIQALKDDPSRSTNVLGEAKKINESQAPK

SEQ ID NO: 274 - protein, 58 aa, synthetic construct
ADNNFNKEQQNAFYEILNMPNLNEEQRNGFIQALKDDPSRSANLLAEAKKINESQAPK

SEQ ID NO: 275 - protein, 58 aa, synthetic construct
ADNKFNKEQQNAFYEILHLPNLNEEQRNGFIQALKDDPSRSANLLAEAKKINDAQAPK

SEQ ID NO: 276 - protein, 58 aa, synthetic construct
ADNKFNKEQQNAFYEILHLPNLTEEQRNGFIQALKDDPSRSKEILAEAKKINDAQAPK

SEQ ID NO: 277 - protein, 58 aa, synthetic construct; identical to SEQ ID NO: 213

SEQ ID NO: 278 - protein, 58 aa, synthetic construct
VDAKFDKEQQNAFYEILHLPNLTEEQRNAFIQALKDDPSRSANLLAEAKKINDAQAPK

SEQ ID NO: 279 - protein, 58 aa, unidentified
IAAQHDKDQQAAFYEILHLPNLTEEQRNAFIQSLRDDPSGSLEILAEAKKVNDAQAPK

SEQ ID NO: 280 - protein, 58 aa, synthetic construct
IAAQHDKEHQAAFYEILHLPNLTEDQRNAFIQSLRDDPSGSLEILGEAKKVNDAQAPK

SEQ ID NO: 281 - protein, 58 aa, synthetic construct
VDNKFNKEQQNAFTEILHLPNLTEEQRNAFIQSLKDDPSGTKEILAEAKKVNDAQAPK

SEQ ID NO: 282 - protein, 56 aa, synthetic construct
AQHDEAQQNAFYQVLNMPNLNADQRNGFIQSLKDDPSGSANVLGEAQKVNDSQAPK

SEQ ID NO: 283 - protein, 61 aa, synthetic construct
ADAQQNKFNKDQQSAFYEILNMPNLNEEQRNGFIQSLKDDPSGSTNVLGEAKKVNESQAPK

SEQ ID NO: 284 - protein, 58 aa, synthetic construct
ADNNFNKEQQNAFYEILNMPNLNEEQRNGFIQSLKDDPSGSANLLAEAKKVNESQAPK

SEQ ID NO: 285 - protein, 58 aa, synthetic construct
ADNKFNKEQQNAFYEILHLPNLNEEQRNGFIQSLKDDPSGSANLLAEAKKVNDAQAPK

SEQ ID NO: 286 - protein, 58 aa, synthetic construct
ADNKFNKEQQNAFYEILHLPNLTEEQRNGFIQSLKDDPSGSKEILAEAKKVNDAQAPK

SEQ ID NO: 287 - protein, 58 aa, synthetic construct; identical to SEQ ID NO: 214

SEQ ID NO: 288 - protein, 58 aa, synthetic construct
VDAKFDKEQQNAFYEILHLPNLTEEQRNAFIQSLKDDPSGSANLLAEAKKVNDAQAPK

SEQ ID NO: 289 - protein, 58 aa, synthetic construct
IAAQHDKDQQAAFYEILHLPNLTEEQRNAFIQALRDDPSGSLEILAEAKKRNDAQAPK

SEQ ID NO: 290 - protein, 58 aa, synthetic construct
IAAQHDKEHQAAFYEILHLPNLTEDQRNAFIQALRDDPSGSLEILGEAKKRNDAQAPK

SEQ ID NO: 291 - protein, 58 aa, synthetic construct
VDNKFNKEQQNAFTEILHLPNLTEEQRNAFIQALKDDPSGTKEILAEAKKRNDAQAPK

SEQ ID NO: 292 - protein, 56 aa, synthetic construct
AQHDEAQQNAFYQVLNMPNLNADQRNGFIQALKDDPSGSANVLGEAQKRNDSQAPK

SEQ ID NO: 293 - protein, 61 aa, synthetic construct
ADAQQNKFNKDQQSAFYEILNMPNLNEEQRNGFIQALKDDPSGSTNVLGEAKKRNESQAPK

SEQ ID NO: 294 - protein, 58 aa, synthetic construct
ADNNFNKEQQNAFYEILNMPNLNEEQRNGFIQALKDDPSGSANLLAEAKKRNESQAPK

SEQ ID NO: 295 - protein, 58 aa, synthetic construct
ADNKFNKEQQNAFYEILHLPNLNEEQRNGFIQALKDDPSGSANLLAEAKKRNDAQAPK

SEQ ID NO: 296 - protein, 58 aa, synthetic construct
ADNKFNKEQQNAFYEILHLPNLTEEQRNGFIQALKDDPSGSKEILAEAKKRNDAQAPK

SEQ ID NO: 297 - protein, 58 aa, synthetic construct; identical to SEQ ID NO: 218

SEQ ID NO: 298 - protein, 58 aa, synthetic construct
VDAKFDKEQQNAFYEILHLPNLTEEQRNAFIQALKDDPSGSANLLAEAKKRNDAQAPK

SEQ ID NO: 299 - protein, 58 aa, synthetic construct
ADNKFNKDHHAAFYEIHNMPYLNEDQHNAAIHSLRHEPSGSLEVLGEAQRVNKSQAPK

SEQ ID NO: 300 - protein, 58 aa, synthetic construct
ADNKFNKAQHNAFYEIHNMPYLNEDQHNAAIHSLRHEPSGSANVLGEAQRVNKSQAPK

SEQ ID NO: 301 - protein, 58 aa, synthetic construct
ADNKFNKAKHNAFYEIHNMPYLNEDQHNGAIHSLRHEPSGSAAVLAEAQRVNKSQAPK

SEQ ID NO: 302 - protein, 58 aa, synthetic construct
ADNKFNKAQHMAFYEIHNMPYLNEDQHNAAIHSLRHEPSGSAEVLAEAQRVNKSQAPK

SEQ ID NO: 303 - protein, 58 aa, synthetic construct
ADNKFNKAQHWAFYEIHNMPYLNEDQHNAAIHSLRHEPSGSAEVLAEAQRVNKSQAPK

SEQ ID NO: 304 - protein, 58 aa, synthetic construct
ADNKFNKAQHTAFYEIHNMPYLNEDQHNAAIHSLRHEPSGSAEVLAEAQRVNKSQAPK

SEQ ID NO: 305 - protein, 58 aa, synthetic construct
ADNKFNKAQHQAFYEIHNMPYLNEDQHNAAIHSLRHEPSGSAEVLAEAQRVNKSQAPK